(19) United States
(12) Patent Application Publication
(10) Pub. No.: US 2024/0100288 A1
(43) Pub. Date: Mar. 28, 2024

(54) HYBRID VENTILATION SYSTEM WITH OXYGEN CONCENTRATOR AND PRESSURIZED OXYGEN SOURCE

(71) Applicant: ZOLL Medical Corporation, Chelmsford, MA (US)

(72) Inventors: Brian P. Harvey, Belmont, MA (US); George Beck, Salem, MA (US); Dorian LeCroy, Parsippany, NJ (US); Denise R. Eizadkhah, Bridgewater, NJ (US); Douglas R. Connelly, Rockaway, NJ (US); Richard L. Pettys, Pearland, TX (US); Richard D. Branson, Buford, SC (US); Rizwan Ajaz, Manvel, TX (US)

(21) Appl. No.: 18/471,122

(22) Filed: Sep. 20, 2023

Related U.S. Application Data
(60) Provisional application No. 63/376,501, filed on Sep. 21, 2022.

(51) Int. Cl. A61M 16/10; A61M 16/00; A61M 16/06

(52) U.S. Cl. CPC A61M 16/101; A61M 16/0003; A61M 16/022; A61M 16/0605; A61M 2202/0208

(57) ABSTRACT

Hybrid ventilation apparatuses, systems and methods are provided. Some example mechanical ventilation apparatuses include a gas delivery system, at least one oxygen concentrator for generating oxygen enriched gas for delivery to a patient, at least one pressurized oxygen source for providing oxygen for delivery to the patient, and a controller. Based at least in part on an FIO2 setting for the gas to be delivered to the patient, the controller may determine an oxygen enriched gas flow rate of the oxygen enriched gas and a pressurized oxygen source flow rate of the oxygen from the at least one pressurized oxygen source, for the gas to be delivered to the patient. The controller may control the gas delivery system to deliver the gas to the patient in accordance with the FIO2 setting, the determined oxygen enriched gas flow rate, and the determined pressurized oxygen source flow rate.

### BACKGROUND

Providing potentially life-saving mechanical ventilation to individuals, such as in out-of-hospital environment, poses many challenges. Such environments may range, for example, from common indoor and outdoor environments to transport, ambulance, military, pandemic, mass casualty and various crisis environments. Responding care providers must sometimes provide life-saving mechanical ventilation using equipment that is taken or carried to the scene, under difficult or unstable circumstances, where there may be one or more patients or victims, sometimes for substantial periods of time, such as hours or days.

The provided mechanical ventilation often requires oxygen supplementation to ensure adequate patient oxygenation, which must be brought to the scene. In some instances, a pressurized oxygen source, such as a high-pressure oxygen tank, may be used. This may have advantages, such as providing a high maximum flow rate and, e.g., a 100% oxygen concentration. However, it may also have disadvantages, such as providing a limited total supply (which can limit the system runtime and/or quantity of the supplied oxygen), presenting size and weight challenges (which may be particularly important for care outside of a hospital), and presenting potential hazards, such as fire or explosion hazards if damaged (which also may be of particular concern in such environments) and, in some cases, potentially increasing risk of patient hyperoxia. More recently, a portable oxygen concentrators (POC) may instead be used. Unlike, for example, a high-pressure oxygen (HPO2) tank or liquid oxygen source, such as a cryogenic liquid oxygen source, a POC may provide an unlimited (that is, self-refreshing) supply. However, a POC may be limited to a maximum rate (per individual POC), and with a limited or lower oxygen concentration (e.g., approximately 93%), which may present a limitation when it is desired to achieve a fraction of inspired oxygen (FIO2) level approaching 100% (that is, higher than the oxygen concentration of gas flow from the POC).

In various environments, such as pre-hospital environments (where, herein, the term “pre-hospital environment” can include any non-hospital environment, whether or not hospital care follows), overcoming challenges such as these to provide effective mechanical ventilation and oxygenation to a patient or patients for the entire potentially substantial required period of time, using equipment that may need to be carried to the scene, often under difficult or unstable circumstances, can be a matter of life or death.

### SUMMARY

One example of an apparatus for providing mechanical ventilation to a patient comprises: a gas delivery apparatus, having a patient interface, configured for gas delivery to the patient; at least one oxygen concentrator, coupled with the gas delivery apparatus, for generating oxygen enriched gas for delivery to the patient via the gas delivery apparatus; at least one pressurized oxygen source, coupled with the gas delivery apparatus, for providing oxygen from the at least one pressurized oxygen source for delivery to the patient via the gas delivery apparatus; and a controller, in communication with the gas delivery apparatus, for causing gas to be delivered to the patient in accordance with a FIO2 setting, the controller being configured to: based at least in part on the FIO2 setting, determine an oxygen enriched gas flow rate of the oxygen enriched gas for the gas to be delivered to the patient and a pressurized oxygen source flow rate of the oxygen from the at least one pressurized oxygen source for the gas to be delivered to the patient, and control the gas delivery apparatus to deliver the gas to the patient in accordance with the FIO2 setting, the determined oxygen enriched gas flow rate, and the determined pressurized oxygen source flow rate.

In some examples, the determination of the oxygen enriched gas flow rate and the pressurized oxygen source flow rate comprises preferentially using the oxygen enriched gas flow rate relative to the pressurized oxygen source flow rate. In some examples the determination of the oxygen enriched gas flow rate and the pressurized oxygen source flow rate comprises using a maximum available oxygen enriched gas flow rate for the oxygen enriched gas flow rate, and supplementing the maximum available oxygen enriched gas flow rate using the pressurized oxygen source flow rate as necessary to achieve the FIO2 setting.

In some examples, the apparatus comprises: a housing; a gas flow generator disposed within the housing; and the gas delivery apparatus, disposed at least partially within the housing, coupled with the gas flow generator, comprising a patient circuit comprising the patient interface, the patient circuit extending from the housing and configured to interface with the patient at least in part for the gas delivery to the patient.

In some examples, the apparatus comprises an oxygen concentrator system coupled with the gas delivery apparatus, wherein the oxygen concentrator system comprises the at least one oxygen concentrator and at least one oxygen concentrator reservoir, wherein the oxygen enriched gas is output from the at least one oxygen concentrator to the at least one oxygen concentrator reservoir, and wherein the oxygen enriched gas is input from the at least one oxygen concentrator reservoir to the gas delivery apparatus.

In some examples, the apparatus comprises at least one oximetry sensor configured to generate signals representative of an oxygen concentration of the patient's blood, wherein the controller is configured to: based at least in part on the signals representative of the oxygen concentration of the patient's blood, determine the FIO2 setting for the gas to be delivered to the patient. In some examples, the FIO2 setting is adjustable, and wherein the controller is configured to determine adjustments to the FIO2 setting based at least in part on patient oxygenation. In some examples, the controller is configured to, based at least in part on the adjustments to the FIO2 setting, make adjustments to the oxygen enriched gas flow rate and the pressurized oxygen source flow rate.

In some examples, the controller is configured to: receive the signals representative of the oxygen concentration of the patient's blood from the at least one oximetry sensor during the delivery of the gas to the patient, based at least in part on the received signals, determine the oxygen concentration of the patient's blood, and based at least in part on the oxygen concentration of the patient's blood, determine the FIO2 setting. In some examples, the controller is configured to adjust the FIO2 setting to maintain patient oxygenation at a desired level or range. In some examples, the controller is configured to utilize closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, the determination of the oxygen enriched gas flow rate and the pressurized oxygen source flow rate comprises determining an overall flow rate of the gas to be delivered to the patient, wherein the overall flow rate comprises the oxygen enriched gas flow rate and the pressurized oxygen source flow rate. In some examples, determining the overall flow rate comprises determining a minute volume. In some examples, the overall flow rate comprises an ambient air flow rate of ambient air for the gas to be delivered to the patient.

In some examples, the oxygen enriched gas flow rate is within a specified range, the determination of the oxygen enriched gas flow rate is based at least in part on the FIO2 setting, an oxygen concentration of the oxygen enriched gas, and an overall flow rate of the gas to be delivered to the patient. In some examples, the specified range comprises a range between a minimum available oxygen enriched gas flow rate and a maximum available oxygen enriched gas flow rate. In some examples, the minimum available oxygen enriched gas flow rate is between 0.1 and 1.0 L/min and the maximum available oxygen enriched gas flow rate is between 2.5 and 3.5 L/min. In some examples, when the oxygen enriched gas flow rate is at a maximum available oxygen enriched gas flow rate, the determination of the pressurized oxygen source flow rate is based at least in part on the FIO2 setting, an oxygen concentration of the oxygen enriched gas, an overall flow rate of the gas to be delivered to the patient, and the oxygen enriched gas flow rate. In some examples, the maximum available oxygen enriched gas flow rate is between 2.5 and 3.5 L/min.

In some examples, the apparatus is portable. In some examples, the gas delivery apparatus is portable. In some examples, the at least one oxygen concentrator is portable. In some examples, the at least one pressurized oxygen source is portable. In some examples, the FIO2 setting is adjustable.

In some examples, the controller is configured to, based at least in part on adjustments to the FIO2 setting, make adjustments to the oxygen enriched gas flow rate and the pressurized oxygen source flow rate. In some examples, the FIO2 setting is adjustable within a range of 21% to 100%. In some examples, the FIO2 setting is manually adjustable. In some examples, the FIO2 setting is adjustable by the controller without requiring manual adjustment. In some examples, the apparatus is configured such that a user of the apparatus can select whether the FIO2 setting is manually adjustable or adjustable by the controller without requiring manual adjustment.

In some examples, the at least one oxygen concentrator has a maximum output oxygen enriched gas flow rate of between 1 L/min and 10 L/min. In some examples, the at least one oxygen concentrator has a maximum output oxygen enriched gas flow rate of between 2 L/min and 4 L/min. In some examples, the at least one oxygen concentrator has a maximum output oxygen enriched gas flow rate of between 2.5 L/min and 3.5 L/min. In some examples, the at least one oxygen concentrator has a maximum output oxygen enriched gas flow rate of 3.0 L/min. In some examples, the at least one oxygen concentrator has a minimum oxygen enriched gas flow rate of between 0 L/min and 0.1 L/min. In some examples, the at least one oxygen concentrator has a minimum oxygen enriched gas flow rate of between 0.1 and 1.0 L/min. In some examples, the at least one oxygen concentrator has a minimum oxygen enriched gas flow rate of between 0.4 and 0.6 L/min. In some examples, the at least one oxygen concentrator has a minimum oxygen enriched gas flow rate of 0.5 L/min.

In some examples, the oxygen enriched gas has an oxygen concentration of between 75% and 98%. In some examples, the oxygen enriched gas has an oxygen concentration of between 90% and 96%. In some examples, the oxygen enriched gas has an oxygen concentration of 93%. In some examples, the at least one oxygen concentrator comprises at least two oxygen concentrators. In some examples, the at least two oxygen concentrators comprises a first oxygen concentrator and a second oxygen concentrator, and determining the gas flow rate of the oxygen enriched gas comprises determining a first gas flow rate for oxygen enriched gas from the first oxygen concentrator and a second gas flow rate for oxygen enriched gas from the second oxygen concentrator. In some examples, the at least one oxygen concentrator comprises at least three oxygen concentrators. In some examples, the at least one pressurized oxygen source comprises at least two pressurized oxygen sources. In some examples, the at least one pressurized oxygen source comprises at least three pressurized oxygen sources. In some examples, the at least one pressurized oxygen source is a high-pressure oxygen source pressurized at a pressure of between 40 and 5,000 psig. In some examples, the at least one pressurized oxygen source comprises at least one liquid oxygen source. In some examples, the at least one pressurized oxygen source comprises at least one pressurized medical oxygen source.

In some examples, the control of the gas delivery apparatus to deliver the gas to the patient comprises actuating at least one valve of the apparatus. In some examples, the control of the gas delivery apparatus to deliver the gas to the patient comprises actuating at least two valves of the apparatus. In some examples, the housing comprises a handle portion configured to enable single handed operation of the apparatus. In some examples, the gas flow generator comprises a blower. In some examples, the gas flow generator comprises a compressor.

In some examples, the controller is configured to detect conditions warranting alerts or alarms to be provided to a user of the apparatus, and to provide the alerts or alarms when the conditions are detected. In some examples, the controller is configured to detect conditions warranting alerts or alarms to be provided to a user of the apparatus, wherein the alerts or alarms comprise at least one of: GUI-based alerts or alarms, and audio alerts or alarms. In some examples, the controller is configured to detect conditions warranting alerts or alarms to be provided to a user of the apparatus, wherein the alerts or alarms relate to detected conditions that pose a potential risk to patient safety. In some examples, the controller is configured to detect conditions warranting alerts or alarms to be provided to a user of the apparatus, and to provide the alerts or alarms when the conditions are detected, wherein the conditions comprise at least one of: a patient hypoxia condition, a patient hypobaric hypoxia condition, a patient desaturation event, two or more patient desaturation events occurring during a predetermined first period of time, a supplemental oxygen requirement that has increased to or above a threshold amount during a second predetermined period of time, the controller losing signaling from an oximetry sensor, the controller losing signaling from the at least one oxygen concentrator, and failure of the at least one oxygen concentrator.

In some examples, the controller is configured such that, for at least some of the detected conditions warranting at least some of the alerts or alarms, operation of the gas delivery apparatus is changed from a normal mode of operation to a fallback mode of operation that is different from the normal mode. In some examples, the fallback mode comprises use of one or more last received valid ventilation related settings. In some examples, the fallback mode comprises disabling closed loop control of patient oxygenation. In some examples, for at least some of the alerts or alarms, visual or audio guidance is determined and provided to the operator of the apparatus. In some examples, the conditions comprise that the two or more patient desaturation events have occurred in a most recent 20 minutes, wherein a first patient desaturation event, of the two or more patient desaturation events, comprises that patient oxygenation has decreased to or below an SpO2 of 88%. In some examples, the conditions comprise that a supplementary oxygen requirement has increased by at least 10% during a most recent 10 minutes. In some examples, the controller is configured to provide the alerts or alarms such that, when the failure of the at least one oxygen concentrator is detected, patient oxygenation from flow from the at least one pressurized oxygen source is used at least in part in place of patient oxygenation from flow from the at least one oxygen concentrator.

In some examples, the oxygen enriched gas has a higher oxygen concentration than that of ambient air. In some examples, the controller comprises at least one processor and at least one memory. In some examples, at least one oxygen sensor external to, and coupled with, the gas delivery apparatus. In some examples, the apparatus comprises at least one oxygen sensor, coupled with an inspiratory line of the patient circuit, for use in measurement of an oxygen concentration of gas being delivered to the patient.

In some examples, determining the oxygen enriched gas flow rate comprises calculating and taking into account a difference between the density of the oxygen enriched gas and a density of air. In some examples, determining a flow rate of gas flow through a compressor of the gas delivery apparatus, wherein the gas flow through the compressor comprises a proportion from the oxygen enriched gas and a proportion from air, wherein determining the flow rate of the gas flow through the compressor comprises calculating and taking into account a difference between the density of the oxygen enriched gas and the density of air. One example of an apparatus for providing mechanical ventilation to a patient, comprises: at least one oximetry sensor configured to generate signals representative of an oxygen concentration of the patient's blood, a gas delivery apparatus, having a patient interface, configured for gas delivery to a patient; at least one oxygen concentrator, coupled with the gas delivery apparatus, for generating oxygen enriched gas for delivery to the patient via the gas delivery apparatus; at least one pressurized oxygen source, coupled with the gas delivery apparatus, for providing oxygen from the at least one pressurized oxygen source for delivery to the patient via the gas delivery apparatus; and a controller, in communication with the gas delivery apparatus, for causing the gas to be delivered to the patient in accordance with a FIO2 setting, the controller being configured to: based at least in part on the signals representative of the oxygen concentration of the patient's blood, determine a FIO2 setting for gas to be delivered to the patient, based at least in part on the FIO2 setting, determine an amount of oxygen supplementation for the gas to be delivered to the patient, comprising determining an oxygen concentrator contribution, to the amount of oxygen supplementation, from the at least one oxygen concentrator and determining a pressurized oxygen source contribution, to the amount of oxygen supplementation, from the at least one pressurized oxygen source, and control the gas delivery apparatus to deliver the gas to the patient in accordance with the FIO2 setting and the determined amount of oxygen supplementation.

In some examples, the oxygen supplementation is from sources of oxygen other than ambient air. In some examples, the determination of the amount of oxygen supplementation for the gas to be delivered to the patient comprises: when the FIO2 setting can be achieved with use of only the oxygen enriched gas for the oxygen supplementation, using, for the oxygen supplementation, only the oxygen enriched gas; and when the FIO2 setting cannot be achieved with use of only the oxygen enriched gas for the oxygen supplementation, using, for the oxygen supplementation, the oxygen enriched gas and the oxygen from the at least one pressurized oxygen source. In some examples, the determination of the amount of oxygen supplementation comprises preferentially using the oxygen concentrator contribution relative to the pressurized oxygen source contribution. In some examples, the determination of the amount of oxygen supplementation comprises using a maximum available oxygen concentrator contribution for the oxygen concentrator contribution, and supplementing the maximum available oxygen concentrator contribution using the pressurized oxygen source contribution as necessary to achieve the FIO2 setting.

In some examples, the apparatus comprises: a housing; a gas flow generator disposed within the housing; and the gas delivery apparatus, disposed at least partially within the housing, coupled with the gas flow generator, comprising a patient circuit comprising the patient interface, the patient circuit extending from the housing and configured to interface with the patient at least in part for the gas delivery to the patient. In some examples, the oxygen supplementation supplements any oxygen provided by any ambient air included in the gas being delivered to the patient. In some examples, the oxygen concentrator contribution is determined to be a zero amount or a non-zero amount, and wherein the pressurized oxygen source contribution from the at least one pressurized oxygen source is determined to be a zero amount or a non-zero amount.

In some examples, the controller is configured to determine the amount of oxygen supplementation based at least in part on an overall flow rate of the gas to be delivered to the patient. In some examples, the determination of the oxygen concentrator contribution comprises determining a flow rate of the oxygen enriched gas. In some examples, the determination of the pressurized oxygen source contribution comprises determining a flow rate of the oxygen from the at least one pressurized oxygen source. In some examples, the at least one oxygen concentrator comprises at least two oxygen concentrators.

In some examples, the apparatus comprises: a housing; a gas flow generator disposed within the housing; and the gas delivery apparatus, disposed at least partially within the housing, coupled with the gas flow generator, comprising a patient circuit comprising the patient interface, the patient circuit extending from the housing and configured to interface with the patient at least in part for the gas delivery to the patient.

In some examples, the controller is configured to cause the gas to be delivered to the patient in accordance with the FIO2 setting, the determined amount of oxygen supplementation, and an overall flow rate for the gas to be delivered to the patient. In some examples, the oxygen concentrator contribution comprises a flow rate of the oxygen enriched gas, wherein the pressurized oxygen source contribution comprises a flow rate of the oxygen from the at least one pressurized oxygen source, wherein the overall flow rate comprises the flow rate of the oxygen enriched gas and the flow rate of the oxygen from the at least one pressurized oxygen source. In some examples, the overall flow rate comprises a flow rate of ambient air. In some examples, the FIO2 setting is adjustable, and wherein the controller is configured to determine adjustments to the FIO2 setting based at least in part on patient oxygenation. In some examples, the controller is configured to, based at least in part on the adjustments to the FIO2 setting, make adjustments to the amount of oxygen supplementation, the oxygen concentrator contribution and the pressurized oxygen source contribution.

In some examples, the controller is configured to: receive the signals representative of the oxygen concentration of the patient's blood from the at least one oximetry sensor during the delivery of the gas to the patient, based at least in part on the received signals, determine the oxygen concentration of the patient's blood, and based at least in part on the oxygen concentration of the patient's blood, determine the FIO2 setting. In some examples, the controller is configured to adjust the FIO2 setting to maintain patient oxygenation at a desired level or range. In some examples, the controller is configured to utilize closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, the apparatus is portable. In some examples, the gas delivery apparatus is portable. In some examples, the at least one oxygen concentrator is portable. In some examples, the at least one pressurized oxygen source is portable. In some examples, the the FIO2 setting is adjustable within a range of 21% to 100%. In some examples, the FIO2 setting is manually adjustable. In some examples, the FIO2 setting is adjustable by the controller without requiring manual adjustment. In some examples, the apparatus is configured such that a user of the apparatus can select whether the FIO2 setting is manually adjustable or adjustable by the controller without requiring manual adjustment.

One example of a system for providing mechanical ventilation to a patient, comprises: a gas delivery system, having a patient interface, configured for gas delivery to the patient; at least one oxygen concentrator, coupled with the gas delivery system, for generating oxygen enriched gas for delivery to the patient via the gas delivery system; at least one pressurized oxygen source, coupled with the gas delivery system, for providing oxygen from the at least one pressurized oxygen source for delivery to the patient via the gas delivery system; and a controller for causing gas to be delivered to the patient in accordance with a FIO2 setting, the controller being configured to: based at least in part on the FIO2 setting, determine an amount of oxygen supplementation for the gas to be delivered to the patient, comprising: when the FIO2 setting can be achieved with use of only the oxygen enriched gas for the oxygen supplementation, using, for the oxygen supplementation, only the oxygen enriched gas, when the FIO2 setting cannot be achieved with use of only oxygen enriched gas for the oxygen supplementation, using, for the oxygen supplementation, the oxygen enriched gas and the oxygen from the at least one pressurized oxygen source, and control the gas delivery system to deliver the gas to the patient in accordance with the FIO2 setting and the determined amount of oxygen supplementation.

In some examples, the determination of the amount of oxygen supplementation comprises preferentially using the oxygen enriched gas for the oxygen supplementation relative to the oxygen from the at least one pressurized oxygen source. In some examples, the determination of the amount of oxygen supplementation comprises using a maximum available amount of the oxygen enriched gas for the oxygen supplementation, and supplementing the maximum available amount of the oxygen enriched gas using the pressurized oxygen from the at least one pressurized oxygen source as necessary to achieve the FIO2 setting. In some examples, the oxygen supplementation supplements any oxygen provided by any ambient air included in the gas being delivered to the patient.

In some examples, the system of claim 83, comprises: a housing; a gas flow generator disposed within the housing; and the gas delivery system, disposed at least partially within the housing, coupled with the gas flow generator, comprising a patient circuit comprising the patient interface, the patient circuit extending from the housing and configured to interface with the patient at least in part for the gas delivery to the patient.

In some examples, the controller is configured to cause the gas to be delivered to the patient in accordance with the FIO2 setting, the determined amount of oxygen supplementation, and an overall flow rate for the gas to be delivered to the patient. In some examples, the amount of oxygen supplementation comprises a flow rate of the oxygen enriched gas and a flow rate of the oxygen from the at least one pressurized oxygen source. In some examples, the overall flow rate comprises a flow rate of ambient air.

In some examples, the system comprises: at least one oximetry sensor configured to generate signals representative of an oxygen concentration of the patient's blood, wherein the controller is configured to: based at least in part on the signals representative of the oxygen concentration of the patient's blood, determine the FIO2 setting for the gas to be delivered to the patient. In some examples, the FIO2 setting is adjustable, and wherein the controller is configured to determine adjustments to the FIO2 setting based at least in part on patient oxygenation.

In some examples, the controller is configured to: receive the signals representative of the oxygen concentration of the patient's blood from the at least one oximetry sensor during the delivery of the gas to the patient, based at least in part on the received signals, determine the oxygen concentration of the patient's blood, and based at least in part on the oxygen concentration of the patient's blood, determine the FIO2 setting. In some examples, the controller is configured to adjust the FIO2 setting to maintain patient oxygenation at a desired level or range. In some examples, the controller is configured to utilize closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, the system is portable. In some examples, the gas delivery system is portable. In some examples, the at least one oxygen concentrator is portable. In some examples, the at least one pressurized oxygen source is portable. In some examples, the FIO2 setting is adjustable. In some examples, the controller is configured to, based at least in part on adjustments to the FIO2 setting, make adjustments to the amount of oxygen supplementation. In some examples, the FIO2 setting is adjustable within a range of 21% to 100%. In some examples, the FIO2 setting is manually adjustable. In some examples, the FIO2 setting is adjustable by the controller without requiring manual adjustment. In some examples, the apparatus is configured such that a user of the apparatus can select whether the FIO2 setting is manually adjustable or adjustable by the controller without requiring manual adjustment. In some examples, the at least one portable oxygen concentrator comprises at least two portable oxygen concentrators.

One example of a method for controlling mechanical ventilation being provided to a patient, comprises: a controller controlling a gas delivery system, of a mechanical ventilation system, to deliver gas to the patient, comprising: causing the gas to be delivered to the patient in accordance with a FIO2 setting, comprising: based at least in part on the FIO2 setting, determining an oxygen enriched gas flow rate of oxygen enriched gas from an oxygen concentrator system, for the gas to be delivered to the patient, and determining a pressurized oxygen source flow rate of oxygen from at least one pressurized oxygen source, for the gas to be delivered to the patient, and controlling the gas delivery system to deliver the gas to the patient in accordance with the FIO2 setting, the determined oxygen enriched gas flow rate, and the determined pressurized oxygen source flow rate.

In some examples, the method comprises determining the oxygen enriched gas flow rate of oxygen enriched gas from the oxygen concentrator system, wherein the oxygen concentrator system comprises at least one oxygen concentrator. In some examples, the method comprises determining the oxygen enriched gas flow rate of oxygen enriched gas from the oxygen concentrator system, wherein the oxygen concentrator system comprises the at least one oxygen concentrator and at least one oxygen concentrator reservoir. In some examples, determining the oxygen enriched gas flow rate and the pressurized oxygen source flow rate comprises preferentially using the oxygen enriched gas flow rate relative to the pressurized oxygen source flow rate. In some examples, determining the oxygen enriched gas flow rate and the pressurized oxygen source flow rate comprises: using a maximum available oxygen enriched gas flow rate for the oxygen enriched gas flow rate, and supplementing the maximum available oxygen enriched gas flow rate using the pressurized oxygen source flow rate as necessary to achieve the FIO2 setting.

In some examples, the method comprises the controller determining the FIO2 setting for the gas to be delivered to the patient based at least in part on signals representative of an oxygen concentration of the patient's blood obtained from at least one oximetry sensor. In some examples, the FIO2 setting is adjustable, and comprising the controller determining adjustments to the FIO2 setting based at least in part on patient oxygenation. In some examples, the controller, based at least in part on the adjustments to the FIO2 setting, making adjustments to the oxygen enriched gas flow rate and the pressurized oxygen source flow rate. In some examples, the method comprises the controller: receiving the signals representative of the oxygen concentration of the patient's blood from the at least one oximetry sensor during the delivery of the gas to the patient, based at least in part on the received signals, determining the oxygen concentration of the patient's blood, and based at least in part on the oxygen concentration of the patient's blood, determining the FIO2 setting. In some examples, the method comprises the controller adjusting the FIO2 setting to maintain patient oxygenation at a desired level or range. In some examples, the method comprises the controller utilizing closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, determining the oxygen enriched gas flow rate and the pressurized oxygen source flow rate comprises determining an overall flow rate of the gas to be delivered to the patient, wherein the overall flow rate comprises the oxygen enriched gas flow rate and the pressurized oxygen source flow rate. In some examples, determining the overall flow rate comprises determining a minute volume. In some examples, determining the overall flow rate comprises an ambient air flow rate of ambient air for the gas to be delivered to the patient. In some examples, the method comprises, when the oxygen enriched gas flow rate is within a specified range, determining the oxygen enriched gas flow rate based at least in part on the FIO2 setting, an oxygen concentration of the oxygen enriched gas, and an overall flow rate of the gas to be delivered to the patient. In some examples, the specified range comprises a range between a minimum available oxygen enriched gas flow rate and a maximum available oxygen enriched gas flow rate. In some examples, the minimum available oxygen enriched gas flow rate is between 0.1 and 1.0 L/min and the maximum available oxygen enriched gas flow rate is between 2.5 and 3.5 L/min. In some examples, the method comprises, when the oxygen enriched gas flow rate is at a maximum available oxygen enriched gas flow rate, determining the pressurized oxygen source gas flow rate based at least in part on the FIO2 setting, an oxygen concentration of the oxygen enriched gas, an overall flow rate of the gas to be delivered to the patient, and the oxygen enriched gas flow rate. In some examples, the maximum available oxygen enriched gas flow rate is between 2.5 and 3.5 L/min.

In some examples, the method comprises the controller causing the gas to be delivered to the patient in accordance with the FIO2 setting, wherein the FIO2 setting is adjustable. In some examples, the method comprises the controller, based at least in part on adjustments to the FIO2 setting, making adjustments to the oxygen enriched gas flow rate and the pressurized oxygen source flow rate. In some examples, the method comprises the controller causing the gas to be delivered to the patient in accordance with the FIO2 setting, wherein the FIO2 setting is adjustable within a range of 21% to 100%. In some examples, the method comprises the controller causing the gas to be delivered to the patient in accordance with the FIO2 setting, wherein the FIO2 setting is manually adjustable. In some examples, the method comprises the controller causing the gas to be delivered to the patient in accordance with the FIO2 setting, wherein the FIO2 setting is adjustable by the controller without requiring manual adjustment. In some examples, the method comprises the controller causing the gas to be delivered to the patient in accordance with the FIO2 setting, comprising the controller, based at least in part on a selection obtained from the from a user of the mechanical ventilation system, determining whether the FIO2 setting is manually adjustable or adjustable by the controller without requiring manual adjustment.

In some examples, the method comprises the controller determining the oxygen enriched gas flow rate, wherein the at least one oxygen concentrator has a maximum output oxygen enriched has flow rate of between 1 L/min and 10 L/min. In some examples, the method comprises the controller determining the oxygen enriched gas flow rate, wherein the at least one oxygen concentrator has a maximum output oxygen enriched has flow rate of between 2 L/min and 4 L/min. In some examples, the method comprises the controller determining the oxygen enriched gas flow rate, wherein the at least one oxygen concentrator has a maximum output oxygen enriched has flow rate of between 2.5 L/min and 3.5 L/min. In some examples, the method comprises the controller determining the oxygen enriched gas flow rate, wherein the at least one oxygen concentrator has a maximum output oxygen enriched has flow rate of 3.0 L/min. In some examples, the method comprises the controller determining the oxygen enriched gas flow rate, wherein the at least one oxygen concentrator has a minimum output oxygen enriched has flow rate of between 0 L/min and 0.1 L/min. In some examples, the method comprises the controller determining the oxygen enriched gas flow rate, wherein the at least one oxygen concentrator has a minimum output oxygen enriched has flow rate of between 0.1 and 1.0 L/min. In some examples, the method comprises the controller determining the oxygen enriched gas flow rate, wherein the at least one oxygen concentrator has a minimum output oxygen enriched has flow rate of between 0.4 and 0.6 L/min. In some examples, the method comprises the controller determining the oxygen enriched gas flow rate, wherein the at least one oxygen concentrator has a minimum output oxygen enriched has flow rate of 0.5 L/min.

In some examples, the method comprises the controller determining the oxygen enriched gas flow rate, wherein the oxygen enriched gas has an oxygen concentration of between 75% and 98%. In some examples, the method comprises controller determining the oxygen enriched gas flow rate, wherein the oxygen enriched gas has an oxygen concentration of between 88% and 94%. In some examples, the method comprises the controller determining the oxygen enriched gas flow rate, wherein the oxygen enriched gas has an oxygen concentration of between 92% and 93%.

In some examples, the method comprises the controller determining the oxygen enriched gas flow rate, wherein the at least one oxygen concentrator comprises at least two oxygen concentrators. In some examples, the method comprises the controller determining the oxygen enriched gas flow rate, wherein the at least one oxygen concentrator comprises at least three oxygen concentrators. In some examples, the method comprises the controller determining the pressurized oxygen source flow rate, wherein the at least one pressurized oxygen source comprises at least two pressurized oxygen sources. In some examples, the method comprises the controller determining the pressurized oxygen source flow rate, wherein the at least one pressurized oxygen source comprises at least three pressurized oxygen sources.

In some examples, the method comprises the controller determining the pressurized oxygen source flow rate, wherein the at least one pressurized oxygen source is a high-pressure oxygen source pressurized at a pressure of between 40-5,000 psig. In some examples, the method comprises the controller determining the pressurized oxygen source flow rate, wherein the at least one pressurized oxygen source comprises at least one liquid oxygen source. In some examples, the method comprises the controller determining the pressurized oxygen source flow rate, wherein the at least one pressurized oxygen source comprises at least one pressurized medical oxygen source.

In some examples, the method comprises the controller causing the gas to be delivered to the patient in accordance with the FIO2 setting, comprising actuating at least one valve of the mechanical ventilation system. In some examples, the method comprises the controller causing the gas to be delivered to the patient in accordance with the FIO2 setting, comprising actuating at least two valves of the mechanical ventilation system. In some examples, the method comprises the controller causing the gas to be delivered to the patient in accordance with the FIO2 setting, comprising actuating at least one blower or compressor.

In some examples, the method comprises the controller detecting conditions warranting alerts or alarms to be provided to a user of the mechanical ventilation system, and providing the alerts or alarms when the conditions are detected. In some examples, the method comprises the controller detecting conditions warranting alerts or alarms to be provided to a user of the mechanical ventilation system, wherein the alerts or alarms comprise at least one of: GUI-based alerts or alarms, and audio alerts or alarms. In some examples, the method comprises the controller detecting conditions warranting alerts or alarms to be provided to a user of the apparatus, wherein the alerts or alarms relate to detected conditions that pose a potential risk to patient safety. In some examples, the method comprises the controller detecting conditions warranting alerts or alarms to be provided to a user of the mechanical ventilation system, and to provide the alerts or alarms when the conditions are detected, wherein the conditions comprise at least one of: a patient hypoxia condition, a patient hypobaric hypoxia condition, a patient desaturation event, two or more patient desaturation events occurring during a predetermined first period of time, a supplemental oxygen requirement that has increased to or above a threshold amount during a second predetermined period of time, the controller losing signaling from the at least one oximetry sensor, the controller losing signaling from the oxygen concentrator system, and failure of the at least one oxygen concentrator.

In some examples, the method comprises the controller, for at least some of the detected conditions warranting at least some of the alerts or alarms, changing operation of the gas delivery system from a normal mode of operation to a fallback mode of operation that is different from the normal mode. In some examples, the method comprises the controller, for at least some of the detected conditions warranting at least some of the alerts or alarms, changing operation of the gas delivery system from the normal mode to the fallback mode, wherein the fallback mode comprises use of one or more last received valid ventilation related settings. In some examples, the method comprises the controller implementing the fallback mode, wherein the fallback mode comprises disabling closed loop control of patient oxygenation. In some examples, the method comprises the controller providing the alerts or alarms, wherein, for at least some of the alerts or alarms, the controller determines and provides visual or audio guidance to a user of the mechanical ventilation system. In some examples, the method comprises the controller providing the alerts or alarms, wherein the conditions comprise that the two or more patient desaturation events have occurred in a most recent 20 minutes, wherein a first patient desaturation event, of the two or more patient desaturation events, comprises that patient oxygenation has decreased to or below an SpO2 of 88%. In some examples, the method comprises the controller providing the alerts or alarms, wherein the conditions comprise that a supplementary oxygen requirement has increased by at least 10% during a most recent 10 minutes. In some examples, the method comprises the controller providing the alerts or alarms such that, when the failure of the oxygen concentrator system is detected, patient oxygenation from flow from the at least one pressurized oxygen source is used at least in part in place of patient oxygenation from flow from the oxygen concentrator system.

In some examples, the method comprises the controller determining the flow rate of the oxygen enriched gas, wherein the oxygen enriched gas has a higher oxygen concentration than that of ambient air.

One example of a method for controlling mechanical ventilation being provided to a patient comprises: a controller controlling a gas delivery system, of a mechanical ventilation system, to deliver gas to the patient, comprising: based at least in part on signals representative of an oxygen concentration of the patient's blood obtained from at least one oximetry sensor, determining a FIO2 setting for the gas to be delivered to the patient; and causing the gas to be delivered to the patient in accordance with the FIO2 setting, comprising: based at least in part on the FIO2 setting, determining an amount of oxygen supplementation for the gas to be delivered to the patient, comprising determining an oxygen concentrator system contribution, of oxygen enriched gas from an oxygen concentrator system, to the amount of oxygen supplementation, and determining a pressurized oxygen source contribution, of oxygen from at least one pressurized oxygen source, to the amount of oxygen supplementation, and controlling the gas delivery system to deliver the gas to the patient in accordance with the FIO2 setting and the determined amount of oxygen supplementation.

In some examples, the method comprises the controller determining the amount of oxygen supplementation, wherein the oxygen supplementation is from sources of oxygen other than ambient air. In some examples, the method comprises the controller determining the oxygen concentrator system contribution, wherein the oxygen concentrator system comprises at least one oxygen concentrator. In some examples, the method comprises determining the oxygen concentrator system contribution, wherein the oxygen concentrator system comprises the at least one oxygen concentrator and at least one oxygen concentrator reservoir.

In some examples, the method comprises the controller causing the gas to be delivered to the patient in accordance with a FIO2 setting, comprising: based at least in part on the FIO2 setting, determining the amount of oxygen supplementation for the gas to be delivered to the patient, comprising: when the FIO2 setting can be achieved with use of only the oxygen enriched gas for the oxygen supplementation, using, for the oxygen supplementation, only the oxygen enriched gas, when the FIO2 setting cannot be achieved with use of only the oxygen enriched gas for the oxygen supplementation, using, for the oxygen supplementation, the oxygen enriched gas and the oxygen from the at least one pressurized oxygen source, and controlling the gas delivery system to deliver the gas to the patient in accordance with the FIO2 setting and the determined amount of oxygen supplementation.

In some examples, the method comprises the controller determining the amount of oxygen supplementation, wherein determining the oxygen concentrator system contribution and the pressurized oxygen source contribution comprises preferentially using the oxygen concentrator system contribution relative to the pressurized oxygen source contribution. In some examples, the method comprises the controller determining the amount of oxygen supplementation, comprising: using a maximum available oxygen concentrator system contribution for the oxygen concentrator system contribution, and supplementing the maximum available oxygen concentrator system contribution using the pressurized oxygen source contribution as necessary to achieve the FIO2 setting. In some examples, the method comprises the controller causing the gas to be delivered to the patient in accordance with the FIO2 setting, wherein the oxygen supplementation supplements any oxygen provided by any ambient air included in the gas being delivered to the patient.

In some examples, the method comprises the controller determining the oxygen concentrator system contribution and the pressurized oxygen source contribution, wherein the oxygen concentrator system contribution is determined to be a zero amount or a non-zero amount, and wherein the pressurized oxygen source contribution from the at least one pressurized oxygen source is determined to be a zero amount or a non-zero amount. In some examples, the method comprises the controller determining the amount of oxygen supplementation for the gas to be delivered to the patient, wherein determining the amount of oxygen supplementation is based at least in part on an overall flow rate of the gas to be delivered to the patient. In some examples, the method comprises the controller determining the amount of oxygen supplementation for the gas to be delivered to the patient, wherein determining the oxygen concentrator system contribution comprises determining a flow rate of the oxygen enriched gas. In some examples, the method comprises the controller determining the amount of oxygen supplementation for the gas to be delivered to the patient, wherein determining the pressurized oxygen source contribution comprises determining a flow rate of the oxygen from the at least one pressurized oxygen source.

In some examples, the method comprises the controller delivering the gas to the patient in accordance with the FIO2 setting and an overall flow rate for the gas to be delivered to the patient. In some examples, the method comprises the controller delivering the gas to the patient in accordance with the FIO2 setting and the overall flow rate, wherein the oxygen concentrator system contribution comprises a flow rate of the oxygen enriched gas, wherein the pressurized oxygen source contribution comprises a flow rate of the oxygen from the at least one pressurized oxygen source, wherein the overall flow rate comprises the flow rate of the oxygen enriched gas and the flow rate of the oxygen from the at least one pressurized oxygen source. In some examples, the method comprises the controller delivering the gas to the patient in accordance with the FIO2 setting and the overall flow rate, wherein the overall flow rate comprises a flow rate of ambient air. In some examples, the FIO2 setting is adjustable, and comprising the controller determining adjustments to the FIO2 setting based at least in part on patient oxygenation.

In some examples, the method comprises the controller: receiving the signals representative of the oxygen concentration of the patient's blood from the at least one oximetry sensor during the delivery of the gas to the patient, based at least in part on the received signals, determining the oxygen concentration of the patient's blood, and based at least in part on the oxygen concentration of the patient's blood, determining the FIO2 setting. In some examples, the method comprises the controller adjusting the FIO2 setting to maintain patient oxygenation at a desired level or range. In some examples, the method comprises the controller utilizing closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range. In some examples, the method comprises the controller causing the gas to be delivered to the patient in accordance with the FIO2 setting, wherein the FIO2 setting is adjustable. In some examples, the method comprises the controller, based at least in part on adjustments to the FIO2 setting, making adjustments to the amount of oxygen supplementation, the oxygen concentrator system contribution and the pressurized oxygen source contribution.

In some examples, the method comprises the controller causing the gas to be delivered to the patient in accordance with the FIO2 setting, wherein the FIO2 setting is adjustable within a range of 21% to 100%. In some examples, the method comprises the controller causing the gas to be delivered to the patient in accordance with the FIO2 setting, wherein the FIO2 setting is manually adjustable. In some examples, the method comprises the controller causing the gas to be delivered to the patient in accordance with the FIO2 setting, wherein the FIO2 setting is adjustable by the controller without requiring manual adjustment. In some examples, the method comprises the controller causing the gas to be delivered to the patient in accordance with the FIO2 setting, comprising the controller, based at least in part on a selection obtained from the from a user of the mechanical ventilation system, determining whether the FIO2 setting is manually adjustable or adjustable by the controller without requiring manual adjustment. In some examples, the oxygen concentrator system comprises at least two oxygen concentrators.

One example of a method for controlling mechanical ventilation being provided to a patient, comprises: a controller controlling a gas delivery system, of a mechanical ventilation system, to deliver gas to the patient, comprising: causing the gas to be delivered to the patient in accordance with a FIO2 setting, comprising: based at least in part on the FIO2 setting, determining an amount of oxygen supplementation for the gas to be delivered to the patient, comprising: when the FIO2 setting can be achieved with use of, for the oxygen supplementation, only oxygen enriched gas from at least one oxygen concentrator, using, for the oxygen supplementation, only the oxygen enriched gas, and when the FIO2 setting cannot be achieved with use of, for the oxygen supplementation, only the oxygen enriched gas from the at least one oxygen concentrator, using, for the oxygen supplementation, the oxygen enriched gas and oxygen from at least one pressurized oxygen source, and controlling the gas delivery system to deliver the gas to the patient in accordance with the FIO2 setting and the determined amount of oxygen supplementation.

In some examples, determining the amount of oxygen supplementation, comprises preferentially using the oxygen enriched gas for the oxygen supplementation relative to the oxygen from the at least one pressurized oxygen source. In some examples, determining the amount of oxygen supplementation comprises: using a maximum available amount of the oxygen enriched gas for the oxygen supplementation, and supplementing the maximum available amount of the oxygen enriched gas using the pressurized oxygen from the at least one pressurized oxygen source as necessary to achieve the FIO2 setting. In some examples, the method comprises the controller causing the gas to be delivered to the patient in accordance with the FIO2 setting, wherein the oxygen supplementation supplements any oxygen provided by any ambient air included in the gas being delivered to the patient. In some examples, the method comprises the controller causing the gas to be delivered to the patient in accordance with the FIO2 setting and an overall flow rate for the gas to be delivered to the patient. In some examples, the method comprises the controller causing the gas to be delivered to the patient in accordance with the FIO2 setting, wherein the amount of oxygen supplementation comprises a flow rate of the oxygen enriched gas and a flow rate of the oxygen from the at least one pressurized oxygen source. In some examples, the method comprises the controller causing the gas to be delivered to the patient in accordance with the FIO2 setting and the overall flow rate, wherein the overall flow rate comprises a flow rate of ambient air.

In some examples, the method comprises the controller determining the FIO2 setting for the gas to be delivered to the patient based at least in part on signals representative of the oxygen concentration of the patient's blood obtained from at least one oximetry sensor. In some examples, the FIO2 setting is adjustable, and comprising the controller determining adjustments to the FIO2 setting based at least in part on patient oxygenation. In some examples, the method comprises the controller: receiving the signals representative of the oxygen concentration of the patient's blood from the at least one oximetry sensor during the delivery of the gas to the patient, based at least in part on the received signals, determining the oxygen concentration of the patient's blood, and based at least in part on the oxygen concentration of the patient's blood, determining the FIO2 setting. In some examples, the method comprises the controller adjusting the FIO2 setting to maintain patient oxygenation at a desired level or range. In some examples, the method comprises the controller utilizing closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, the method comprises the controller causing the gas to be delivered to the patient in accordance with the FIO2 setting, wherein the FIO2 setting is adjustable. In some examples, the method comprises the controller, based at least in part on adjustments to the FIO2 setting, making adjustments to the amount of oxygen supplementation. In some examples, the method comprises the controller causing the gas to be delivered to the patient in accordance with the FIO2 setting, wherein the FIO2 setting is adjustable within a range of 21% to 100%. In some examples, the method comprises the controller causing the gas to be delivered to the patient in accordance with the FIO2 setting, wherein the FIO2 setting is manually adjustable. In some examples, the method comprises the controller causing the gas to be delivered to the patient in accordance with the FIO2 setting, wherein the FIO2 setting is adjustable by the controller without requiring manual adjustment. In some examples, the method comprises the controller causing the gas to be delivered to the patient in accordance with the FIO2 setting, comprising the controller, based at least in part on a selection obtained from the from a user of the mechanical ventilation system, determining whether the FIO2 setting is manually adjustable or adjustable by the controller without requiring manual adjustment. In some examples, the at least one oxygen concentrator comprises at least two oxygen concentrators.

One example of an apparatus for providing mechanical ventilation to a patient, comprises: a gas delivery apparatus, having a patient interface, configured for gas delivery to the patient; at least one oxygen concentrator, coupled with the gas delivery apparatus, for generating oxygen enriched gas for delivery to the patient via the gas delivery apparatus; and a controller, in communication with the gas delivery apparatus, for causing gas to be delivered to the patient in accordance with a FIO2 setting, the controller being configured to: based at least in part on the FIO2 setting, determine an oxygen enriched gas flow rate of the oxygen enriched gas for the gas to be delivered to the patient, and control the gas delivery apparatus to deliver the gas to the patient in accordance with the FIO2 setting and the determined oxygen enriched gas flow rate.

In some examples, the apparatus comprises at least one pressurized oxygen source, coupled with the gas delivery apparatus, for providing oxygen from the at least one pressurized oxygen source for delivery to the patient via the gas delivery apparatus, and wherein the controller is configured to: based at least in part on the FIO2 setting, determine the oxygen enriched gas flow rate of the oxygen enriched gas for the gas to be delivered to the patient and a pressurized oxygen source flow rate of the oxygen from the at least one pressurized oxygen source for the gas to be delivered to the patient, and control the gas delivery apparatus to deliver the gas to the patient in accordance with the FIO2 setting, the determined oxygen enriched gas flow rate, and the determined pressurized oxygen source flow rate.

In some examples, the determination of the oxygen enriched gas flow rate and the pressurized oxygen source flow rate comprises preferentially using the oxygen enriched gas flow rate relative to the pressurized oxygen source flow rate. In some examples, the determination of the oxygen enriched gas flow rate and the pressurized oxygen source flow rate comprises using a maximum available oxygen enriched gas flow rate for the oxygen enriched gas flow rate, and supplementing the maximum available oxygen enriched gas flow rate using the pressurized oxygen source flow rate as necessary to achieve the FIO2 setting.

In some examples, the apparatus comprises a housing; a gas flow generator disposed within the housing; and the gas delivery apparatus, disposed at least partially within the housing, coupled with the gas flow generator, comprising a patient circuit comprising the patient interface, the patient circuit extending from the housing and configured to interface with the patient at least in part for the gas delivery to the patient. In some examples, the apparatus comprises an oxygen concentrator system coupled with the gas delivery apparatus, wherein the oxygen concentrator system comprises the at least one oxygen concentrator and at least one oxygen concentrator reservoir, wherein the oxygen enriched gas is output from the at least one oxygen concentrator to the at least one oxygen concentrator reservoir, and wherein the oxygen enriched gas is input from the at least one oxygen concentrator reservoir to the gas delivery apparatus.

In some examples, the apparatus comprises at least one oximetry sensor configured to generate signals representative of an oxygen concentration of the patient's blood, wherein the controller is configured to: based at least in part on the signals representative of the oxygen concentration of the patient's blood, determine the FIO2 setting for the gas to be delivered to the patient. In some examples, the FIO2 setting is adjustable, and wherein the controller is configured to determine adjustments to the FIO2 setting based at least in part on patient oxygenation. In some examples, the controller is configured to, based at least in part on the adjustments to the FIO2 setting, make adjustments to the oxygen enriched gas flow rate and the pressurized oxygen source flow rate. In some examples, the controller is configured to: receive the signals representative of the oxygen concentration of the patient's blood from the at least one oximetry sensor during the delivery of the gas to the patient, based at least in part on the received signals, determine the oxygen concentration of the patient's blood, and based at least in part on the oxygen concentration of the patient's blood, determine the FIO2 setting. In some examples, the controller is configured to adjust the FIO2 setting to maintain patient oxygenation at a desired level or range. In some examples, the controller is configured to utilize closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

In some examples, the determination of the oxygen enriched gas flow rate and the pressurized oxygen source flow rate comprises determining an overall flow rate of the gas to be delivered to the patient, wherein the overall flow rate comprises the oxygen enriched gas flow rate and the pressurized oxygen source flow rate. In some examples, determining the overall flow rate comprises determining a minute volume. In some examples, the overall flow rate comprises an ambient air flow rate of ambient air for the gas to be delivered to the patient. In some examples, the oxygen enriched gas flow rate is within a specified range, the determination of the oxygen enriched gas flow rate is based at least in part on the FIO2 setting, an oxygen concentration of the oxygen enriched gas, and an overall flow rate of the gas to be delivered to the patient. In some examples, the specified range comprises a range between a minimum available oxygen enriched gas flow rate and a maximum available oxygen enriched gas flow rate. In some examples, the minimum available oxygen enriched gas flow rate is between 0.1 and 1.0 L/min and the maximum available oxygen enriched gas flow rate is between 2.5 and 3.5 L/min. In some examples, when the oxygen enriched gas flow rate is at a maximum available oxygen enriched gas flow rate, the determination of the pressurized oxygen source flow rate is based at least in part on the FIO2 setting, an oxygen concentration of the oxygen enriched gas, an overall flow rate of the gas to be delivered to the patient, and the oxygen enriched gas flow rate. In some examples, the maximum available oxygen enriched gas flow rate is between 2.5 and 3.5 L/min.

In some examples, the apparatus is portable. In some examples, the gas delivery apparatus is portable. In some examples, the at least one oxygen concentrator is portable. In some examples, the at least one pressurized oxygen source is portable. In some examples, the FIO2 setting is adjustable. In some examples, the controller is configured to, based at least in part on adjustments to the FIO2 setting, make adjustments to the oxygen enriched gas flow rate and the pressurized oxygen source flow rate. In some examples, the FIO2 setting is adjustable within a range of 21% to 100%. In some examples, the FIO2 setting is manually adjustable. In some examples, the FIO2 setting is adjustable by the controller without requiring manual adjustment. In some examples, the apparatus is configured such that a user of the apparatus can select whether the FIO2 setting is manually adjustable or adjustable by the controller without requiring manual adjustment.

In some examples, the at least one oxygen concentrator has a maximum output oxygen enriched gas flow rate of between 1 L/min and 10 L/min. In some examples, the at least one oxygen concentrator has a maximum output oxygen enriched gas flow rate of between 2 L/min and 4 L/min. In some examples, the at least one oxygen concentrator has a maximum output oxygen enriched gas flow rate of between 2.5 L/min and 3.5 L/min. In some examples, the at least one oxygen concentrator has a maximum output oxygen enriched gas flow rate of 3.0 L/min. In some examples, the at least one oxygen concentrator has a minimum oxygen enriched gas flow rate of between 0 L/min and 0.1 L/min. In some examples, the at least one oxygen concentrator has a minimum oxygen enriched gas flow rate of between 0.1 and 1.0 L/min. In some examples, the at least one oxygen concentrator has a minimum oxygen enriched gas flow rate of between 0.4 and 0.6 L/min. In some examples, the at least one oxygen concentrator has a minimum oxygen enriched gas flow rate of 0.5 L/min. In some examples, the oxygen enriched gas has an oxygen concentration of between 75% and 98%. In some examples, the oxygen enriched gas has an oxygen concentration of between 90% and 96%. In some examples, the oxygen enriched gas has an oxygen concentration of 93%.

In some examples, the at least one oxygen concentrator comprises at least two oxygen concentrators. In some examples, the at least one oxygen concentrator comprises at least three oxygen concentrators. In some examples, the at least one pressurized oxygen source comprises at least two pressurized oxygen sources. In some examples, the control of the gas delivery apparatus to deliver the gas to the patient comprises actuating at least one valve of the apparatus. In some examples, the control of the gas delivery apparatus to deliver the gas to the patient comprises actuating at least two valves of the apparatus. In some examples, the housing comprises a handle portion configured to enable single handed operation of the apparatus. In some examples, the gas flow generator comprises a blower. In some examples, the gas flow generator comprises a compressor. In some examples,

In some examples, the controller is configured to detect conditions warranting alerts or alarms to be provided to a user of the apparatus, and to provide the alerts or alarms when the conditions are detected. In some examples, the controller is configured to detect conditions warranting alerts or alarms to be provided to a user of the apparatus, wherein the alerts or alarms comprise at least one of: GUI-based alerts or alarms, and audio alerts or alarms. In some examples, wherein the controller is configured to detect conditions warranting alerts or alarms to be provided to a user of the apparatus, wherein the alerts or alarms relate to detected conditions that pose a potential risk to patient safety. In some examples, the controller is configured to detect conditions warranting alerts or alarms to be provided to a user of the apparatus, and to provide the alerts or alarms when the conditions are detected, wherein the conditions comprise at least one of: a patient hypoxia condition, a patient hypobaric hypoxia condition, a patient desaturation event, two or more patient desaturation events occurring during a predetermined first period of time, a supplemental oxygen requirement that has increased to or above a threshold amount during a second predetermined period of time, the controller losing signaling from an oximetry sensor, the controller losing signaling from the at least one oxygen concentrator, and failure of the at least one oxygen concentrator.

In some examples, the controller is configured such that, for at least some of the detected conditions warranting at least some of the alerts or alarms, operation of the gas delivery apparatus is changed from a normal mode of operation to a fallback mode of operation that is different from the normal mode. In some examples, the fallback mode comprises use of one or more last received valid ventilation related settings. In some examples, the fallback mode comprises disabling closed loop control of patient oxygenation. In some examples, wherein, for at least some of the alerts or alarms, visual or audio guidance is determined and provided to the operator of the apparatus. In some examples, the conditions comprise that the two or more patient desaturation events have occurred in a most recent 20 minutes, wherein a first patient desaturation event, of the two or more patient desaturation events, comprises that patient oxygenation has decreased to or below an SpO2 of 88%. In some examples, the conditions comprise that a supplementary oxygen requirement has increased by at least 10% during a most recent 10 minutes. In some examples, the controller is configured to provide the alerts or alarms such that, when the failure of the at least one oxygen concentrator is detected, patient oxygenation from flow from the at least one pressurized oxygen source is used at least in part in place of patient oxygenation from flow from the at least one oxygen concentrator. In some examples, the oxygen enriched gas has a higher oxygen concentration than that of ambient air. In some examples, the controller comprises at least one processor and at least one memory.

In some examples, the apparatus comprises at least one oxygen sensor external to, and coupled with, the gas delivery apparatus. In some examples, the apparatus comprises at least one oxygen sensor, coupled with an inspiratory line of the patient circuit, for use in measurement of an oxygen concentration of gas being delivered to the patient. In some examples, the at least one oxygen concentrator comprises at least two oxygen concentrators.

In some examples, determining the oxygen enriched gas flow rate comprises calculating and taking into account a difference between the density of the oxygen enriched gas and a density of air. In some examples, determining a flow rate of gas flow through a compressor of the gas delivery apparatus, wherein the gas flow through the compressor comprises a proportion from the oxygen enriched gas and a proportion from air, wherein determining the flow rate of the gas flow through the compressor comprises calculating and taking into account a difference between the density of the oxygen enriched gas and the density of air.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples. The figures are incorporated in and constitute a part of this specification, but are not intended to limit the scope of the disclosure. In the figures, identical or nearly identical components that are illustrated in various figures may be represented by like numerals. For purposes of clarity, not every component may be labeled in every figure.

FIG. 1 illustrates an example emergency care environment including a hybrid ventilation system including a portable ventilator, a portable oxygen concentrator (POC) and a pressurized oxygen cylinder.

FIG. 2 illustrates an example hybrid ventilation system including a portable ventilator, a portable oxygen concentrator and a pressurized oxygen cylinder.

FIG. 3 is a block diagram illustrating components of an example hybrid ventilation system used in providing mechanical ventilation to a patient, including a set of oxygen supplementation sources and a mechanical ventilation device.

FIGS. 4-6 are flow diagrams illustrating example methods of FIO2 control using a hybrid ventilation system including a POC and a pressurized oxygen source.

FIGS. 7-8 are block diagrams illustrating examples of contributing sources of gas flow in a hybrid ventilation system.

FIG. 9 is a table illustrating an example of parameters associated with use of a hybrid ventilation system.

FIGS. 10A-B are tables illustrating examples of gas sources and aspects relating to flow rates associated with regions of operation of Type A and Type B hybrid ventilation systems, respectively.

FIG. 11A-B are plots illustrating examples of regions of operation and supplemental oxygen source gas flows for a Type A hybrid ventilation system, corresponding with FIG. 10A, for POCs with minimum flow rates of 0.5 L/min and 0.01 L/min.

FIG. 11C-D are plots illustrating examples of regions of operation and supplemental oxygen source gas flows for a Type B hybrid ventilation system, corresponding with FIG. 10B, for POCs with minimum flow rates of 0.5 L/min and 0.01 L/min.

FIGS. 12A-D are plots illustrating oxygen source contributions for Type A and Type B hybrid ventilation systems, corresponding with FIGS. 11A-D, respectively, for a minute volume of 2.0 L/min.

FIG. 12E is a plot illustrating oxygen source contributions for a Type A1 hybrid ventilation system, for a POC with a minimum flow rate of 0.5 L/min, for a minute volume of 2.0 L/min.

FIGS. 13A-D are plots illustrating oxygen source contributions for Type A and Type B hybrid ventilation systems, corresponding with FIGS. 11A-D, respectively, for a minute volume of 8.0 L/min.

FIGS. 14A-D are plots illustrating oxygen source contributions for Type A and Type B hybrid ventilation systems, corresponding with FIGS. 11A-D, respectively, for a minute volume of 16.0 L/min.

FIG. 15 is a plot illustrating total oxygen flow across a range of FIO2 settings and minute volumes, for an example hybrid ventilation system.

FIGS. 16A-D are plots illustrating example POC flow rates across a range of FIO2 settings and minute volumes, corresponding with FIGS. 11A-D, respectively.

FIGS. 17A-B are plots illustrating example high-pressure oxygen (HPO2) source oxygen contributions, for Type A and Type B hybrid ventilation systems, with FIG. 17A corresponding to FIGS. 11A-B and FIG. 17B corresponding to FIGS. 11C-D.

FIG. 17C is a plot illustrating example HPO2 source oxygen contributions, for a Type A1 hybrid ventilation system.

FIG. 18 is a plot illustrating example ambient air oxygen contributions, for a Type A hybrid ventilation system, corresponding to FIGS. 11A-B.

FIGS. 19A-D are plots illustrating example FIO2 setting versus delivered FIO2, across a range of FIO2 settings and minute volumes, for Type A and Type B hybrid ventilation systems, corresponding with FIGS. 11A-D, respectively.

FIGS. 20A-B are plots illustrating additional system runtime of a 660 L HPO2 source provided by use of a Type A hybrid ventilation system versus a HPO2 source-only ventilation system, across a range of FIO2 settings and minute volumes, corresponding with FIGS. 11A-B.

FIGS. 20C-D are plots illustrating additional system runtime of a 660 L HPO2 source provided by use of a Type B hybrid ventilation system versus a HPO2 source-only ventilation system, across a range of FIO2 settings and minute volumes, corresponding with both of FIGS. 11C-D.

FIG. 20E is a plot illustrating additional system runtime of a 660 L HPO2 source provided by use of a Type A1 hybrid ventilation system versus a HPO2 source-only ventilation system, across a range of FIO2 settings and minute volumes.

FIGS. 21A-C are plots illustrating example flow rates and ranges by region for a Type A hybrid ventilation system, corresponding with FIG. 11A, for minutes volumes of 3.5, 8.0 and 16 L/min, respectively.

FIG. 22 is a plot illustrating FIO2 settings achievable and not achievable using a system with a POC but no HPO2 source, while all FIO2 settings are achievable using some example hybrid ventilation systems.

FIG. 23 is a plot illustrating settings achievable using a system with two POCs but no HPO2 source, while all FIO2 settings are achievable using some example hybrid ventilation systems.

FIGS. 24-26 include plots relating to performed animal studies illustrating use of a hybrid ventilation system to achieve and maintain normoxia after lung injury, at ground level, at a simulated altitude of 8,000 feet, and at a simulated altitude of 16,000 feet, respectively.

FIG. 27 is a block diagram illustrating example software architecture related aspects of a hybrid ventilation system including a software-based FIO2 control with ventilation and PEEP control including closed loop control (CLC).

FIG. 28 is a block diagram of an example portable ventilator that can be used in a hybrid ventilation system.

FIG. 29 is a block diagram illustrating example aspects of a mechanical ventilation apparatus of a portable ventilator that can be used in a hybrid ventilation system.

FIG. 30 is an illustration of a simplified example portable ventilator and display and user interface, with FIO2 closed loop control, which can be used in a hybrid ventilation system.

FIG. 31 is an illustration of a simplified example ventilation related display and user interface, which can be used in a hybrid ventilation system.

FIG. 32 illustrates an example plot showing linear variation of a gas density correction factor with an increasing FIO2 of gas through a compressor of a ventilator of a hybrid ventilation system.

FIG. 33 illustrates example aspects of patient circuits that can be used in a hybrid ventilation system.

FIG. 34 is a block diagram illustrating example components of various devices described with reference to preceding figures.

### DETAILED DESCRIPTION

Some embodiments provide techniques, apparatuses, systems and methods for use in providing mechanical ventilation to a patient, including in prehospital environments, which may include, for example, transport, ambulance, military, pandemic, mass casualty or various crisis environments, and may pose many challenges. Portable equipment may be required, which the care provider may need to transport or carry to the scene. Furthermore, the environment may be chaotic, distracting, crowded, or unstable (e.g., a military, crisis or disaster environment). Potentially using portable equipment, the care provider may need to provide mechanical ventilation to one or several patients, sometimes for a substantial period of time, such as hours or even days. The size, footprint, weight, safety and complexity of the equipment or its use may present difficulties, inefficiencies or risks, potentially in transport to the scene, at the scene itself and during the providing of care.

Providing potentially life-saving mechanical ventilation to a patient often requires providing gas to the patient with a higher oxygen (O2) concentration than that of ambient air, to assure sufficient patient oxygenation. This, in turn, may require that supplemental oxygen be supplied for the provided gas (where supplemental oxygen does not include ambient air).

One source of supplemental oxygen for mechanical ventilation is pressurized oxygen, such as may be provided using a high-pressure oxygen (HPO2) tank or a liquid oxygen source, such as a cryogenic liquid oxygen source. For example, a pressured oxygen source may be pressurized, at a pressure of, e.g., between 40-5,000 psig, 40-3,000 psig, 40-1,000 psig, 40-500 psig, 40-90 psig, 50-60 psig or 55 psig. In providing supplemental oxygen, for example, a HPO2 source, such as a HPO2 tank, may have various advantages. It may provide 100% oxygen, or nearly 100%, oxygen, which allows achieving very high FIO2 settings, and it may be capable of providing a high flow rate.

However, a HPO2 tank, for example, may have various disadvantages, as well. It may be subject to risk of fire or explosion if impacted, punctured or damaged, which may be a particularly significant consideration in a prehospital, e.g., military or crisis, environment.

Additionally, a HPO2 tank, for example, may be of significant size, weight and footprint, which may make portability and/or use at a non-hospital scene, along with other equipment, more difficult or complicated. For instance, in military operations, the HPO2 supply may constitute, e.g., up to 30% of the total weight of a patient care system, thus potentially presenting a significant limitation. Moreover, it provides a limited supply of oxygen, depending on the capacity of the tank (or tanks) and the rate of flow used over time. Furthermore, greater capacity adds size and weight, which may increase the difficulty of portability, transportation and use. HPO2 supply limitation may be a particularly significant consideration in prehospital environment, for example, where life-saving mechanical ventilation may need to be provided for a substantial period of time, and/or to multiple patients, and where predictability or certainty with regard to needed supply may be at least initially be lacking.

Another more recently available and source of supplemental oxygen for mechanical ventilation is an oxygen concentrator, or portable oxygen concentrator (POC). In operation, a POC may, for example, input ambient air, and separate and remove nitrogen from the air to increase the concentration of oxygen in output gas flow, such as to 93%, or, e.g., approximately 90-96% oxygen. In providing supplemental oxygen, a POC also has advantages and disadvantages.

A POC may introduce less risk, such as risk of fire or explosion, than, for example, an HPO2 source. Additionally, a POC can effectively supply a continuous, unlimited duration of flow and an unlimited total amount of output oxygen-concentrated gas. However, a POC may be limited in the flow rate of the output gas that it can provide, such as to a maximum of, e.g., approximately 3.0 L/min. Furthermore, the output gas may be, e.g., 93% oxygen, rather than, e.g., approximately 100% oxygen, such as may be the case with an HPO2 tank, for example. The limited flow rate and limited oxygen concentration of the output gas from the POC may, in some cases, be insufficient oxygen supplementation to ensure adequate or optimal patient oxygenation, or to ensure it for all required periods of time and FIO2 settings. For example, patients experiencing severe respiratory distress may require a very high FIO2 for substantial periods of time, which may not be achievable (or sufficiently close to achievable) using only a POC (or even several POCs) for oxygen supplementation.

Furthermore, in some cases, the POC may have a minimum flow rate for output gas that is too high to provide or precisely provide some FIO2 settings, such as low FIO2 settings (though greater than that of ambient air, so that oxygen supplementation is needed) at low minute volumes, which, for example, may be particularly relevant for pediatric patients. In such cases, even use of the minimum flow rate from the POC may overshoot, to a degree, a required FIO2 setting (which may or may not be considered acceptable in various situations, given, e.g., the patient and their disease state). Diversion of a selected portion of the flow from the POC, away from delivery to the patient, may allow providing effective POC flow rates, for gas delivered to the patient, below the minimum flow rate for the POC, but may complicate or decrease optimization of the system or its use (e.g., decrease battery run time).

Additionally, in a system using only a POC for oxygen supplementation, it may be desired or needed to turn the POC on or off frequently, or to rapidly increase or decrease flow rate from the POC, or even to go from zero flow to maximum flow as rapidly as possible, which may present difficulties. For example, the POC may have a significant “warm up” period after being turned on before it can produce its full oxygen concentration and flow rate.

Some embodiments provide techniques, apparatuses, systems, methods, algorithms and software that address, and provide solutions to technical problems associated with, the use of one or more oxygen sources such as pressurized oxygen sources, and/or the use of one or more oxygen concentrators or POCs, for providing supplemental oxygen during mechanical ventilation. Some embodiments include, or include use of, a hybrid ventilation system. The hybrid ventilation system may include a mechanical ventilator, at least one oxygen concentrator, such as a POC, and at least one pressurized oxygen source, such as a HPO2 tank. One or both of the POC and the HPO2 tank may be used to provide supplemental oxygen (e.g., gas with an oxygen concentration greater than that of ambient air) during the providing of mechanical ventilation to a patient or patients. In some embodiments, flow from the POC may be preferentially used, where possible or practical, relative to flow from the HPO2 tank, so as to conserve the HPO2 tank supply.

Additionally, some embodiments provide techniques, apparatuses, systems, methods, algorithms and software that provide solutions to technical problems associated with, for example, optimizing operation of a hybrid ventilation system, such as by optimizing flow rates of output gas from the HPO2 tank and the POC delivered to the patient. In some embodiments, one or more algorithms, which may be stored and executed on one or more controllers of a portable ventilator, elsewhere, or both, may be used in determining and optimizing flow rates from the POC and the HPO2 tank, such as based at least in part on a current FIO2 setting.

Furthermore, in various embodiments, FIO2 may be manually adjusted, or one or more algorithms may be used that utilize closed loop control (CLC) (e.g., physiological closed loop control (PCLC)) of parameters such as FIO2 (FIO2 CLC) so that FIO2 does not need to be manually adjusted. Furthermore, in some embodiments, both manual FIO2 adjustment and FIO2 CLC may be available or used at different times, such as by user selection (whether on a ventilator or other connected device in a hybrid ventilation system) or by system determination or optimization. For example, in FIO2 CLC, FIO2 may be determined or adjusted by a controller of the portable ventilator (or other local or remote device(s)), such as based at least in part on measured patient peripheral oxygen saturation (SpO2), without requiring manual adjustment of the FIO2. In some embodiments, for example, FIO2 CLC may increase the efficiency of supplemental oxygen use during the providing of the mechanical ventilation, e.g., by automatically titrating the FIO2 to prevent excessive oxygen administration, which could lead to hyperoxia. Additionally, use of FIO2 CLC may simplify operation of the portable ventilator or the hybrid ventilation system, increasing ease of use, decreasing required attention or time of the user of the ventilator, and/or decreasing potential distractions for the user, which may include, in some cases, a less or minimally trained user in a prehospital environment. Furthermore, in some embodiments, use of FIO2 CLC may allow optimization of flow rates from, e.g., the POC and/or the HPO2 source, which may include optimizing preferential use of flow from the POC relative to flow from the HPO2 source. This optimization may, in turn, allow greater system optimization, such as may include greater conservation of the HPO2 supply (or use of a smaller HPO2 supply), which may allow longer system runtime, more optimal system use, or more optimal patient oxygenation.

Additionally, in some embodiments, a portable ventilator of a hybrid ventilation system may have other capabilities that may be particularly advantageous in various non-hospital settings. For example, in some embodiments, the portable ventilator may be capable of either internal or remote control, or of switching between the two. This may allow use of a portable ventilator with less controls, and that may be smaller, lighter or simpler to operate, providing advantages in minimizing the spatial impact of the portable ventilator in the potentially crowded space immediately surrounding the patient in a non-hospital setting.

Furthermore, in some embodiments, the portable ventilator may be capable of monitoring and assessing one or more respiratory parameters of the patient, which may be used in determining a respiratory status of the patient and in determining appropriate patient treatment. This may be especially advantageous in non-hospital settings, where the availability of other devices at the scene may be limited or unpredictable.

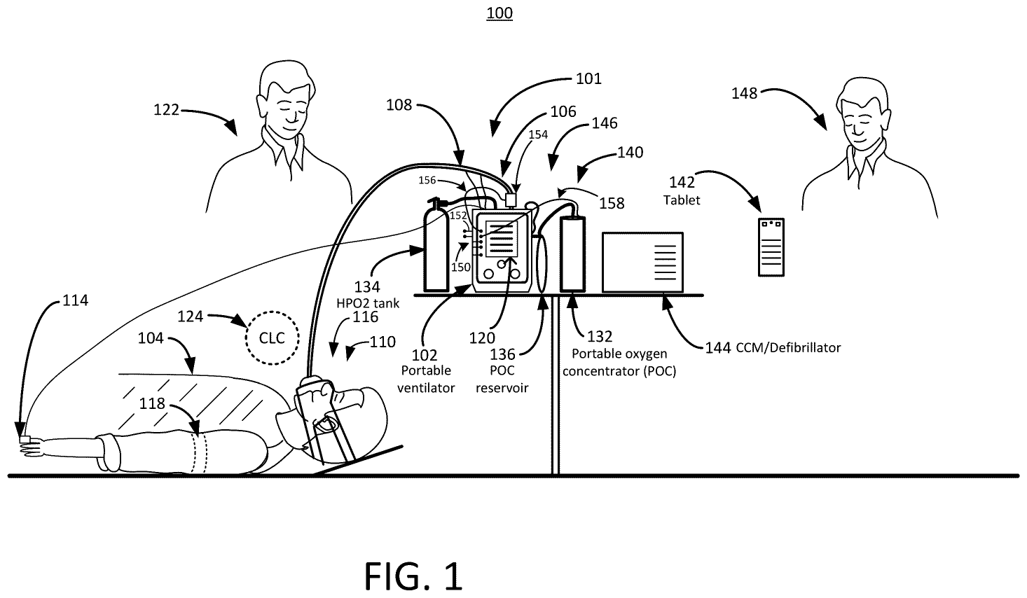

FIG. 1 illustrates an example emergency care environment 100 including a hybrid ventilation system 101. The hybrid ventilation system 101 includes a portable ventilator 106, a portable oxygen concentrator (POC) system 146 that includes one or more POCs 132 and one or more POC reservoirs 136 (although, in some embodiments, no POC reservoir is included), and a pressurized oxygen source, such as a high-pressure oxygen (HPO2) source, e.g., a HPO2 container, such as a HPO2 tank 134. While the depicted hybrid ventilation system 101, and components thereof, are portable (e.g., may be carried or transported, or practically carried or transported, to a non-hospital location or setting), in some embodiments, a hybrid ventilation system, and/or particular components thereof, are not portable. As depicted, the environment 100 also includes user 122 of the portable ventilator 102, and may or may not include one or more additional local or remote care providers, such as the depicted user 148 of the tablet 142.

In various embodiments, various devices and systems may be communicatively coupled by wired and/or wireless connection to each other, which may include the hybrid ventilation system 101 and the portable ventilator 106. In various embodiments, many different combinations of devices and systems, and roles for each, may be included or used. As shown, a CCM/defibrillator 144 and the tablet computing device 142 are included.

In various embodiments, the portable ventilator 106 may be internally controlled, remote controlled (e.g., by a local or non-local device, system or platform other than the portable ventilator 106), or may be capable of both, or of switching or being switched between them. In some embodiments, the portable ventilator 106 is capable of being remote controlled by or from, e.g., a critical care monitor (CCM), defibrillator, CCM/defibrillator (such as CCM/defibrillator 144), medical or monitoring device, computing device, portable computing device, tablet (such as tablet 142), smartphone, system, facility, medical care or monitoring facility, or hospital.

In various embodiments, particular sensing capabilities and components may be included and distributed in various ways between various devices or systems. These may include, for example, one or more electrodes, capnographic sensors, pulse oximeters, flow sensors, pneumotachometers, spirometers, pressure sensors, barometric sensors, humidity sensors, oxygen sensors, temperature sensors, electrical or magnetic sensors, light/electromagnetic spectrum/optical sensors, blood pressure monitors, heart rate monitors, electrocardiogram (ECG) sensors, and others. Sensing capabilities and components may relate, for example, to patient parameters and various patient systems, such as respiratory and circulatory systems. Sensing capabilities and components may also relate to parameters associated with devices and their operation, including, for example, the portable ventilator 106 or other ventilation system, CCM/defibrillator 144, CCM, electrotherapy system, defibrillator, or pacing system. In some embodiments, any of various sensing capabilities, such as those of the portable ventilator 106, CCM/defibrillator 144, or tablet 142, may instead be provided by one or more other devices or systems, or may be distributed between multiple devices or systems. Also, in various embodiments, various sensing components, or all or part of the patient circuit 108, may or may not be considered to be part of a portable ventilator, or other device, even if they are connected thereto. Furthermore, in various embodiments, roles and functions of the various devices or systems may be distributed differently between the devices or systems.

The portable ventilator 106 (and/or one or more other components, devices or systems of the environment 100) may include various sensing, measuring, computerized, electrical, mechanical, coupling and output components. As depicted, the portable ventilator 106 includes an oximetry sensor 114, such as a pulse oximeter or other sensor for providing a direct or indirect measurement, estimation or indication of oxygen saturation (SpO2) or other blood oxygen content or concentration related parameter, a capnographic sensor 116 or capnograph, and a blood pressure sensor/monitor 118, and may also include one or more flow sensors such as pneumotachometers, or pressure sensors, among other things.

As depicted, the portable ventilator 106 includes a display and user interface 120 that may provide data relating to various patient physiological, respiratory and ventilation related parameters, and may include other output or presentation components, such as a speaker. In some embodiments, the display and user interface 120, or other output devices, may provide a display that is integrated to include data relating to operation of other coupled devices that may also be in use with the patient, such as, for example, a defibrillator. The display and user interface 120 may also allow user interaction, including to obtain or display data, change settings, such as, in some embodiments, a FIO2 setting, accept suggested or recommended settings changes, or view or respond to alarms or alerts, among other things.

In some embodiments, the portable ventilator 106 is capable of providing closed loop control (CLC) 124 of one or more ventilation or patient related parameters, such as FIO2 (FIO2 CLC) or Positive End Expiratory Pressure or Baseline Airway Pressure CLC (PEEP CLC or BAP CLC). However, in some embodiments, FIO2 CLC, PEEP CLC, or other CLC is not included. For example, in some embodiments, a user may select or input a FIO2 setting, and/or adjust the flow rate of oxygen or supplemental oxygen, based at least in part on current patient SpO2, which may be monitored by one or more devices of the environment 100. Furthermore, in some embodiments, a user may turn on or off CLC, such as FIO2 CLC, select whether CLC is used, or select whether CLC is used at a particular time or during a particular time period.

The setting depicted in FIG. 1 may be a non-hospital setting, but, in various embodiments, hybrid ventilation systems are provided for use in hospital or non-hospital settings. In some embodiments, some or all of the components of the hybrid ventilation system 101, including the portable ventilator 106, the POC system 140 and/or the HPO2 tank 134, may be transported to the setting to provide emergency care to a patient 104.

When delivering mechanical ventilation, the portable ventilator 106 may provide breathing gas to the patient 104 via a mechanical ventilation apparatus that includes a gas flow generator, such as a blower or a compressor, and a gas delivery apparatus including a patient circuit 108 that includes a facemask 110. However, in some embodiments, ventilation may be provided via intubation or other patient interface such as nasal cannula rather than via a facemask 110.

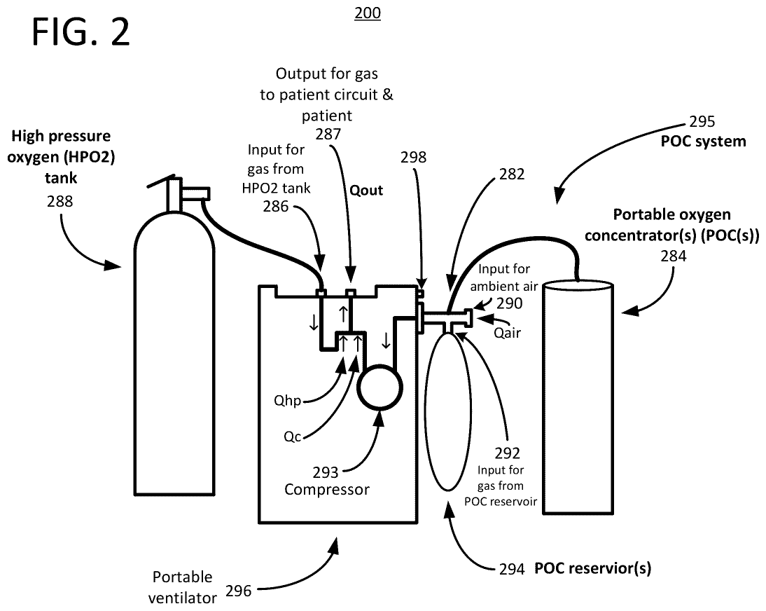

As depicted, the POC system 140 includes a POC reservoir, such as a POC reservoir bag 136. As described in further detail in FIG. 2, in the embodiment depicted, POC output gas flow is captured in the reservoir bag 136. The captured POC output gas from the reservoir bag 136 may then be used to provide gas flow into the ventilator 106. The flow rate of the gas from the reservoir bag 136 into the ventilator 106 may be determined or regulated by a controller of the ventilator (or one or more other controlling sources).

In some embodiments, FIO2 CLC may be used in the control of the output of the POC system 140 and/or the HPO2 tank 134 during the providing of mechanical ventilation. In some embodiments, control, including of FIO2 CLC and/or PEEP CLC, may be provided internally by the portable ventilator 106 or by one or more other devices, such as one or more remote control devices for the portable ventilator 106, such tablet 142 or CCM/defibrillator 144.

In some embodiments, the portable ventilator 106 includes sensors such as one or more flow sensors, pneumotachometers or pressure sensors, which may include sensors disposed within the patient circuit 108 and/or within the portable ventilator 106, for sensing signals representative of gas flow or pressure within the gas delivery apparatus of the portable ventilator 106 and/or to the patient 104. In some embodiments, one or more of the sensors may be coupled with or part of one or more spirometers, for example. In some embodiments, a controller of the portable ventilator 106 receives the signals representative of the gas flow or pressure. The controller may control aspects of mechanical ventilation provided by the portable ventilator 106 based at least in part on the signals representative of gas flow or pressure. Furthermore, in some embodiments, based at least in part on the signals representative of the gas flow or pressure, the controller may generate respiratory parameter data corresponding with at least one respiratory parameter of the patient, such as, for example, respiratory system compliance (Crs), respiratory system resistance (Rrs), vital capacity (VC), forced vital capacity (FVC), forced expiratory volume (FEV) at a timed interval such as FEV1, forced expiratory flow (FEF), or FEF25-75, peak expiratory flow rate (PEF or PEFR), maximal voluntary ventilation (MVV) or others. In some embodiments, a controller of the portable ventilator may cause transmission of generated respiratory parameter data to be received (directly or via one or more intermediary entities) by other local or non-local devices or systems in the environment 100, e.g., the CCM/defibrillator 144, the tablet 142, or elsewhere, such as for use in determining a respiratory status of the patient 104. However, in some embodiments, the portable ventilator 106 may determine, or participate in determining, the respiratory status of the patient.

As depicted in FIG. 1, the ventilator 106 also includes a number of ports, such as flexible use external component connection or data ports, herein referred to as smart ports 150. Each of the smart ports 150 may, for example, be configured to facilitate coupling or connection, via, e.g., cabling, and integration of any of various types of external components or modules, or for receiving and facilitating monitoring of various types of data, regarding, for example, FIO2, pulse oximetry, flow rate, pressure, humidity, or others. In some embodiments, each of the smart ports 150 may be associated with an insert/cover 152. For any unconnected smart port 150, the associated insert/cover 152 may be inserted into the smart port to maintain ingress protection when, for example, no cable is connected to the smart port. Furthermore, as depicted in FIG. 1, an oxygen sensor (O2 sensor) 154 may be coupled with some portion of the ventilation system 101 or ventilator 106. As depicted, the O2 sensor is connected externally to the ventilator 106 and coupled with the inspiratory limb of the patient circuit 108. The O2 sensor is also connected by cable 156 to a smart port 150 (or otherwise), and is used in sensing, measurement and monitoring of the oxygen concentration of gas being delivered to the patient 104. However, in other embodiments, one or more oxygen sensors can also be coupled in various other locations of the ventilation system 101 or ventilator, and may be used in sensing, measurement and monitoring oxygen concentrations elsewhere in, or in association with, the ventilation system 108, whether internal or external to the ventilator 106 itself.

In particular, in the embodiment shown in FIG. 1, the oxygen sensor 154 (or oxygen sensing module that includes an oxygen sensor) can be used in monitoring to determine whether/confirm that the FIO2 of gas being delivered to the patient is in fact the expected or set FIO2. This can be useful or critical in, e.g., identifying, troubleshooting, providing alarms and/or making corrections (whether automatically or manually) in the event that the actual delivered FIO2 of gas being delivered to the patient does not match the expected FIO2. For example, when the POC 132 is being used, if the FIO2 of the gas being delivered to the patient is less than expected, this could indicate that there is a failure associated with operation of the POC 132, that there is an occlusion at an inlet from the POC system 140 to the ventilator 106, or that the POC reservoir 134 is disconnected from the inlet to the ventilator 106. In turn, for example, associated alerts, alarms, messages, guidance, instructions, or troubleshooting information can be provided to a user of a device or system of the hybrid ventilation system 101.

Additionally, in the embodiment shown in FIG. 1, the POC 132 is coupled by cable 158 to one of the smart ports 150 for receipt of data from the POC 132 regarding POC related parameters, including POC output gas flow rate and oxygen concentration. If, for example, the parameters, such as flow rate, are not as expected or are otherwise outside of acceptable ranges, then alarms, alerts, messages, etc., may be provided to a user. Additionally, if the communication from the POC 132 is cut off, appropriate alerts, alarms, messages, etc. may be provided to a user.

It is noted that, in some embodiments, such as in a hybrid ventilation system including a number of devices and/or systems, a wired or wireless data connection may be provided between the POC 132 and/or HPO2 tank 134, and one or more device or systems other than, or in addition to, the ventilator 106. Data communicated by the POC 132 or POC system 140, and by the HPO2 tank 134 (or other pressurized oxygen source) may allow detection of, e.g., whether each is connected to the ventilator 106, whether each is turned off or on, and what flow rate of gas is being provided, for example, as well as other parameters.

FIG. 2 illustrates a simplified example hybrid ventilation system 200 including a portable ventilator 296, a HPO2 tank 288, and a POC system 295 including a POC 284 (or multiple POCs) and a POC reservoir 294 (or several), such as a reservoir bag. The POC system 295 also includes any other devices and components, such as may include intermediary, connection or coupling devices or components, such as may be used in coupling gas flow from the POC 284 directly or indirectly to an inlet of the ventilator 296 such that POC gas flow enters the ventilator 296. The portable ventilator 296 includes a compressor 293, an output port 287 for gas to the patient circuit and the patient, an input port 286 for output gas flow from the HPO2 tank 288, and an input port 282 for gas flow from the POC reservoir(s) 294 of the POC system 295, as well as ambient air. Inputs 292, 290 are provided to the input port 282, including the input 292 for gas flow from the POC system 295 and the input 290 for ambient air flow. In some embodiments, however, the POC system 295 may not include a reservoir 294, or the reservoir(s) 294 may not, or may not always, be used. In such embodiments or instances, for example, an input 298 may be provided (in addition to, or instead of, input 292) for input of gas from the POC 284 directly, rather than via a reservoir 294, for example.

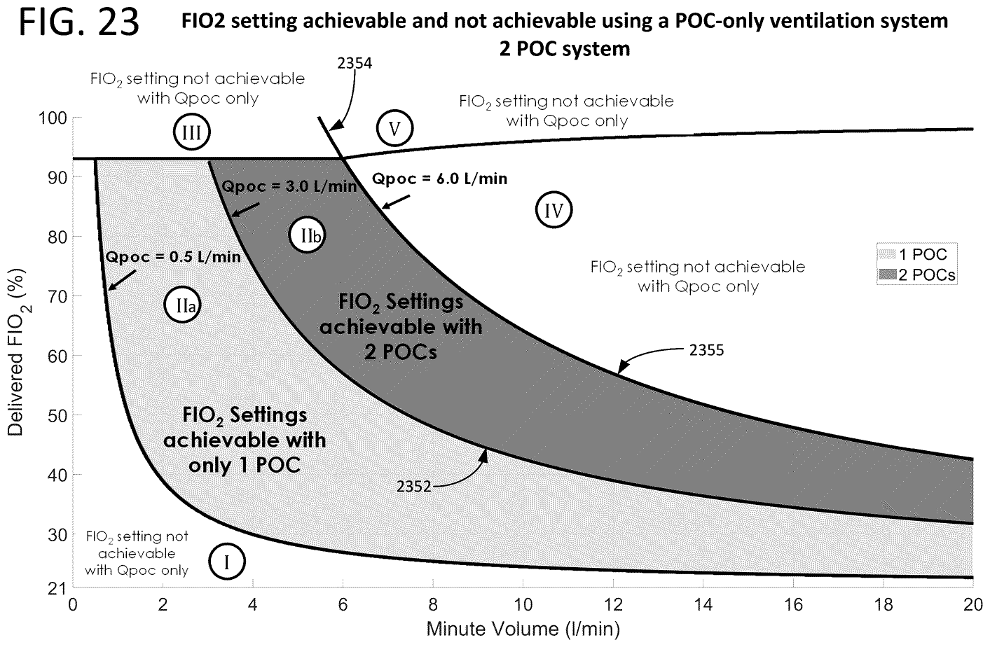

Although a single POC is depicted in FIG. 2, in some embodiments, more than one POC may be included, as further described herein, including with reference to FIG. 23. In multiple POC embodiments, each POC may be coupled with the input port 282, for example, or multiple input ports. For example, in various embodiments, outputs from each of the POCs may be merged prior to the coupling to the input port 282, or may separately be input, or in other ways. As described further herein, including with reference to FIG. 23, in some embodiments, outputs from each of, or some of, multiple POCs may be controlled by or within the hybrid ventilation system 200.

As depicted in FIG. 2, flow of ambient air into the portable ventilator 296 via input 290 is represented as Qair. Gas flow from the HPO2 tank 288 into the portable ventilator 296 via input 286 is represented as Qhp. Gas flow from the compressor 293 is represented as Qc, where Qc includes the gas flow of ambient air, represented as Qair, and the gas flow from the POC system 295, via the POC reservoir 294 of the POC system 295, represented as Qpoc.

Output gas to the patient circuit and the patient (whether or not all such gas actually reaches the patient) is represented as Qout, which is made up of any contributions from each of Qpoc, Qhp and Qair (any of which may be non-zero or zero at a particular time).

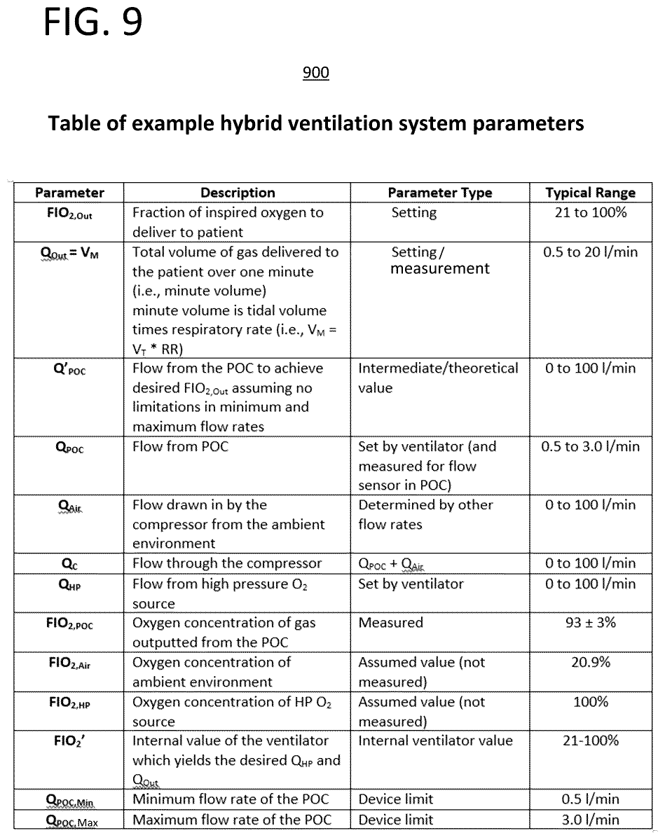

In example embodiments described herein, the example parameters listed in table 900 of FIG. 9 and used in equations 1-12 as described herein, including Qout, Q′poc, Qpoc, Qair, Qc, Qhp, Qpoc, min and Qpoc, max, may refer to average flow rates, such as over the period of time of one breath or multiple breaths. For example, various flow rates may be non-zero during an inspiratory period (when gas may be taken into the ventilator and delivered from the ventilator to the patient circuit and the patient), but zero during an expiratory period (when no gas may be taken into the ventilator and delivered to the patient circuit and the patient). Generally, however, rates and flow rates, as used herein, can include instantaneous rates and average rates over various periods of time.

It is noted that, in some embodiments, in hybrid ventilation systems that include a POC reservoir, during various periods of operation, the POC may output gas continuously to the POC reservoir, during both inspiratory and expiratory periods, which gas is captured by the POC reservoir. Generally, during an inspiratory period (and while the POC outputs gas that is captured by the POC reservoir), captured gas may flow from the POC reservoir into the ventilator, drawn through the compressor, and flow out of the ventilator to the patient circuit and the patient, such that the POC reservoir may be emptied at the end of the inspiratory period. During an expiratory period, when no gas may be delivered to the patient, and no gas may be input from the POC reservoir into the ventilator, the POC may continue to output gas, which output gas is captured by, and stored by, the POC reservoir (which may be emptied at the start of the expiratory period) until the following inspiratory period. As such, at any given time, the flow rate from the POC (Qpoc) may not equal the flow rate from the POC reservoir, and the flow rate from the POC reservoir may not be limited by any minimum or maximum flow rate of the POC. However, generally, over a period of time, such as the period of time of one or multiple breaths (where each breath includes one inspiration period and one expiration period), the average flow rate from the POC (Qpoc) may be equal to the average flow rate from the POC reservoir, and the total volume of gas output from the POC may be equal the total volume of gas output from the POC reservoir, consistent with the principal of conservation of mass.

Particularly, the controller of the ventilator may control including the following. During an expiration, no flow from the POC reservoir may be input to the ventilator (or output to the patient), but the POC outputs flow that is stored in the POC reservoir (which may be emptied at the start of the expiration). During an inspiration, stored gas from the POC reservoir is input to the ventilator, while the POC continues to output flow that is received by the POC reservoir. During an inspiration, for example, the flow rate of the gas input by the POC reservoir to the ventilator may be controlled (potentially, in some instances or for some periods, at a flow rate greater than the maximum flow rate of the POC), so that the POC reservoir empties at the end of the inspiration period.

In some embodiments, a POC system may include two or more POCs, both of which may be used simultaneously. In such embodiments, each of the POCs may contribute to the POC flow rate (Qpoc), so that Qpoc is made up of the combined flow from all of the POCs. Similarly, in some embodiments, two or more HPO2 sources may be used, where each contributes to the HPO2 flow rate (Qhp).

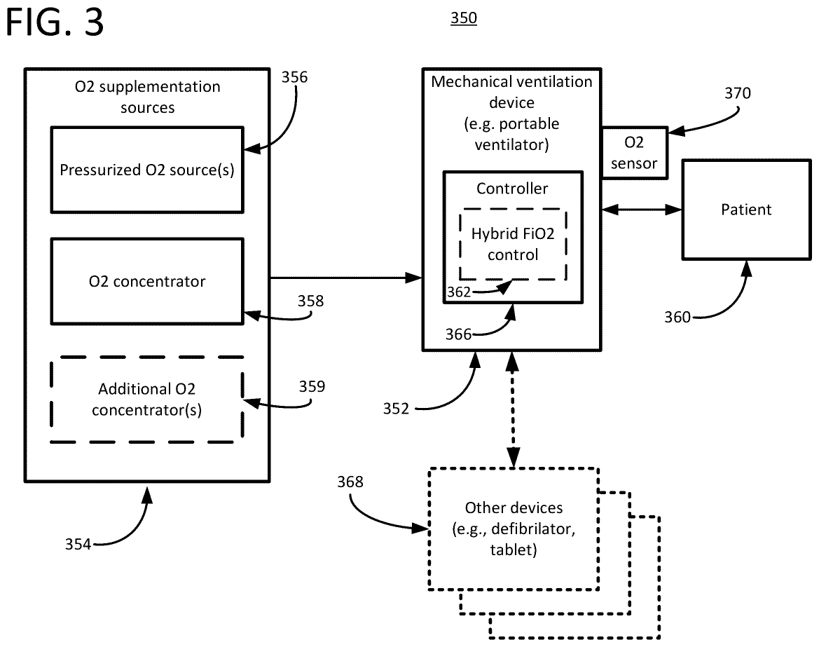

FIG. 3 is a block diagram illustrating components of an example hybrid ventilation system 350 used in providing mechanical ventilation to a patient 360. The hybrid ventilation system 350 includes a mechanical ventilation device such as a portable ventilator 352 and a set of oxygen supplementation sources 354, for providing oxygen supplementation for use in mechanical ventilation provided to a patient 360. In some embodiments, the system 350 also includes an O2 sensor 370 (or O2 sensing component including an O2 sensor), as described, for example, with reference to FIG. 1, that may be, e.g., coupled with the inspiration limb of the patient circuit, or elsewhere. The environment may also include various other devices 368, such may include as a CCM/defibrillator or tablet, among other devices, some or all which may be coupled with the portable ventilator 352 through wired or wireless coupling. The oxygen supplementation sources 354 may include a pressurized oxygen source 356, such as a HPO2 tank, and an oxygen concentrator 358, such as a POC. The portable ventilator 352 includes a controller 366 that includes a software-based hybrid FIO2 control 362. The hybrid FIO2 control 362 may, for example, include one or more algorithms used in implementing various embodiments described herein, such as in implementing various hybrid ventilation apparatuses, systems, methods and techniques. Although the hybrid FIO2 control 362 is depicted within the controller 366 of the portable ventilator 352, in various embodiments, a hybrid FIO2 control, or components thereof, may be stored and/or executed in part or in whole elsewhere, such as in one or more other devices, systems or platforms that may be coupled with the portable ventilator 352, which may or may not include a local or non-local remote control device, system or platform for the portable ventilator 352.

In some embodiments, a hybrid ventilation system may include a single POC 358. However, in some embodiments, one or more additional POCs 359 may be included. In some embodiments in which multiple POCs are included, a controller or controllers of the system controls a flow rate, and/or an amount of oxygen supplementation, from each of the multiple POCs, such as to reach the total required oxygen supplementation or enriched oxygen gas flow rate from the POCs. In various embodiments and implementations, the flow rates from the POCs may be the same or different between any or each of the multiple POCs. Details of some embodiments including more than one POC are provided herein, including with reference to FIG. 23.

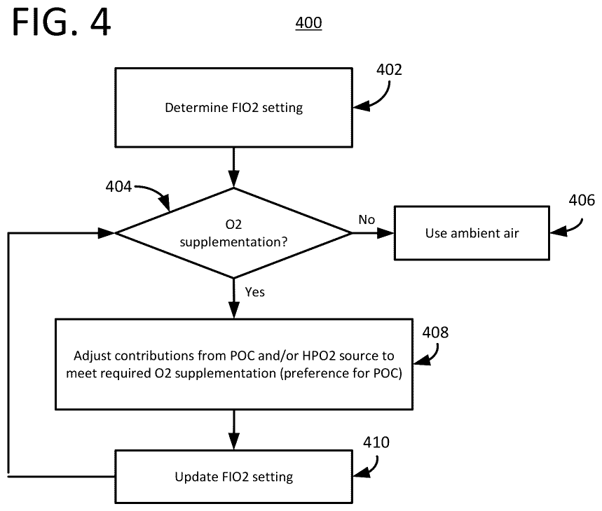

FIGS. 4-6 are flow diagrams illustrating example methods 400, 500, 600 of FIO2 control using a hybrid ventilation system including a POC system and a pressurized oxygen source. In some embodiments, the methods 400, 500, 600 may be implemented using a hybrid FIO2 control, such as the hybrid FIO2 control 362 as depicted in FIG. 3.

In FIG. 4, at step 402, the method 400 determines a FIO2 setting (e.g., 30%, 50%, etc.) for gas to be delivered to the patient during the providing of mechanical ventilation to the patient. At step 404, the method 400 determines whether oxygen supplementation is required in providing gas in accordance with the FIO2 setting. If not, then, at step 406, ambient air is used, without oxygen supplementation. However, if oxygen supplementation is required, then, at step 408, the method 400 adjusts the contributions from a POC and/or a HPO2 source to meet required oxygen supplementation needs based on the FIO2 setting, with a preference for using, or using as much as possible, flow from the POC relative to flow from the HPO2 source. It is noted that adjustments for other reasons may also be used, such as to maintain a desired minute volume. At step 410, the method 400 updates the FIO2 setting, such as by manual updating or by use of FIO2 CLC. Following step 410, the method 400 returns to step 404, where it is determined whether oxygen supplementation is required based at least in part on the updated FIO2 setting. Steps 404-410 are then repeated, as appropriate, as the FIO2 setting is repeatedly or continuously updated and may be adjusted, and appropriate oxygen supplementation is provided and adjusted as needed over time.

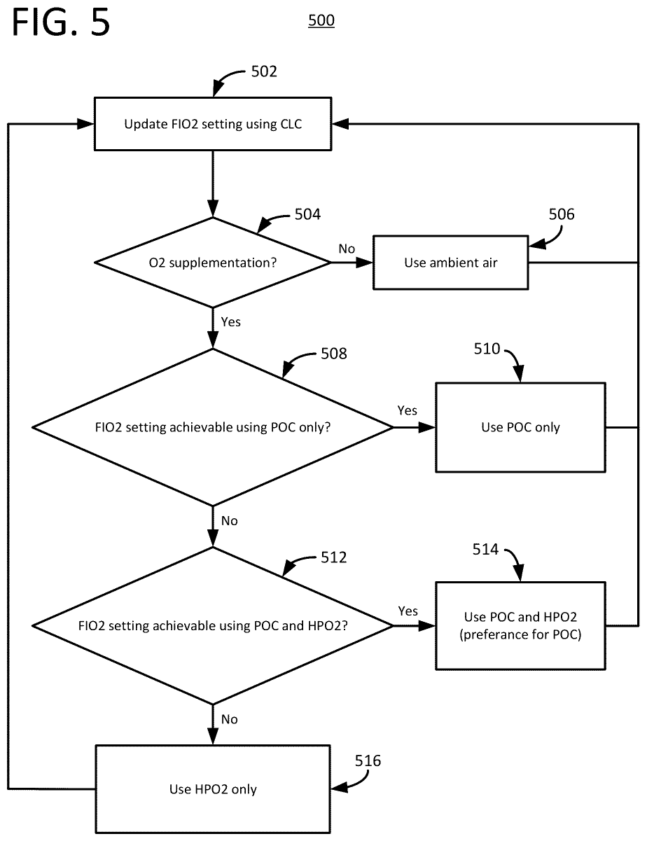

In FIG. 5, at step 502, the method 500 updates a FIO2 setting using FIO2 CLC. At step 504, the method 500 determines whether oxygen supplementation is required in providing gas in accordance with the FIO2 setting. If not, then, at step 506, ambient air is used, without oxygen supplementation. However, if oxygen supplementation is required, then, at step 508, the method 500 determines whether the current FIO2 setting is achievable using oxygen supplementation only from POC flow, without use of HPO2 flow. If so, then, at step 510, POC flow only is used for oxygen supplementation, without use of HPO2 gas flow.

If, at step 508, the method 500 determines that the current FIO2 setting is not achievable using oxygen supplementation only from the POC, then, at step 512, the method 500 determines whether the FIO2 setting is achievable using both POC flow and HPO2 flow for oxygen supplementation. If so, then, at step 514, both POC flow and HPO2 flow are used for oxygen supplementation, with a preference for use of POC flow. If the current FIO2 setting is not achievable using both POC flow and HPO2 flow for oxygen supplementation, then, at step 516, HPO2 flow only is used for oxygen supplementation.

It is noted, however, that, in various embodiments as described herein, under certain specified conditions, even if, using POC flow, a certain POC flow rate, a maximum or minimum POC flow rate, or POC flow without HPO2 flow, requires, to a degree, undershooting the FIO2 setting (achieving a FIO2 under the FIO2 setting) or, to a degree, overshooting the FIO2 setting (achieving a FIO2 over the FIO2 setting), the POC flow may nonetheless be utilized for oxygen supplementation, with or without use of additional HPO2 flow. For example, in some embodiments, under certain conditions that may include very high or very low FIO2 settings and particular minute volumes or ranges of minute volumes, POC flow, a certain POC flow rate, a maximum or minimum POC flow rate, or POC flow without HPO2 flow may be used, even if it requires undershooting or overshooting the FIO2 setting, or undershooting or overshooting the FIO2 setting by no more than a specified or acceptable amount or percentage. For example, in some embodiments, undershooting or overshooting, to a degree, the FIO2 setting may be used in some circumstances if any disadvantages of doing so, such as with regard to reducing optimization or efficiency of patient treatment or overall system operation, may be considered absent, of less magnitude than, or outweighed by any advantages of doing so, such as may include conserving HPO2 supply or use, and simplifying the system or its operation.

For example, in some cases or embodiments, use of POC flow, or a particular POC flow rate, even if undershooting and/or overshooting the FIO2 setting may occur, may provide an advantage by allowing reduced use of the HPO2 supply, thus conserving and potentially extending the availability of the HPO2 supply. Conserving HPO2 supply, for example, may be particularly important if extensive or extended use of HPO2 supply is anticipated, and/or if HPO2 supply is small or low. Furthermore, in some cases, use of POC flow, or a particular POC flow rate, even if it requires or sometimes requires undershooting or overshooting the FIO2 setting, may also provide an advantage by reducing difficulties or inefficiencies that may be created by additional system or system use complexity that may otherwise be required. Such complexities may include, for example, frequent POC turn on or turn off, rapid ramp up or ramp down of POC flow or oxygen concentration, or diversion of a portion of POC flow so that it does not enter the ventilator or get delivered to the patient. However, in some cases or embodiments, the advantage provided by achieving particular FIO2 settings, without undershooting or overshooting them, may outweigh the disadvantages, such as in situations in which HPO2 supply is high or not anticipated to be exhausted, or when frequent turn on or turn off of the POC, or rapid ramp up or ramp down of the POC, is not considered likely, problematic or very problematic, or when patient treatment requires achieving the FIO2 settings without undershooting or overshooting them, for example.

Accordingly, various embodiments as described herein may include delivering gas to a patient in accordance with an FIO2 setting. In various embodiments, or in various circumstances or at various times, this may include delivering gas to the patient without overshooting or undershooting the FIO2 setting, or may include delivering gas to the patient including overshooting or undershooting the FIO2 setting, or including overshooting or undershooting the FIO2 setting to a particular or specified degree, amount or proportion.

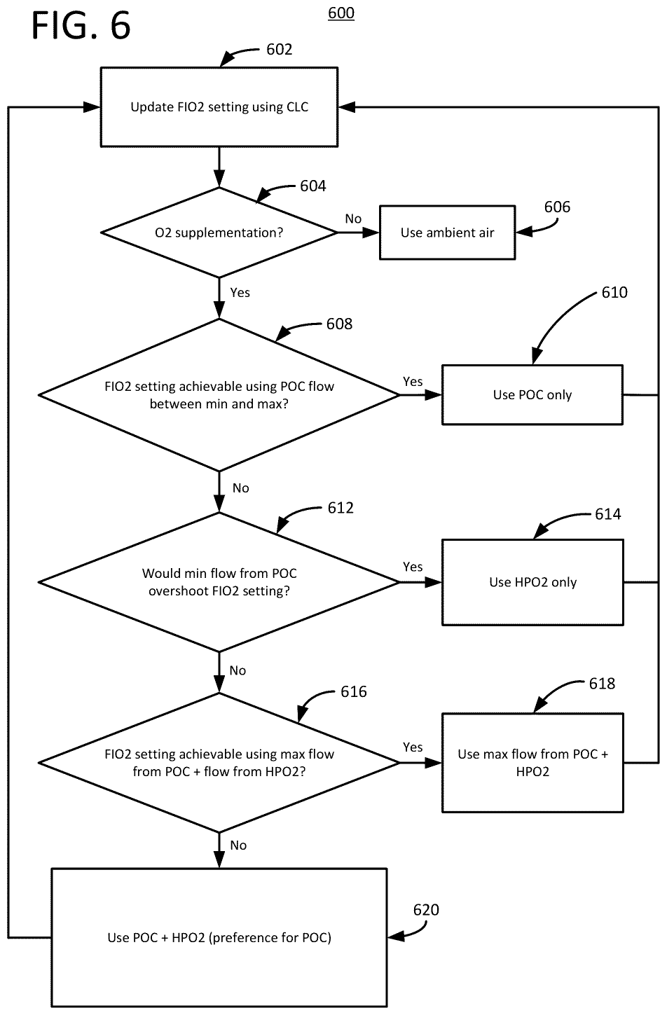

In FIG. 6, at step 602, the method 600 updates a FIO2 setting using FIO2 CLC. At step 604, the method 600 determines whether oxygen supplementation is required in providing gas in accordance with the FIO2 setting. If not, then, at step 606, ambient air is used, without oxygen supplementation.

At step 604, if the method 600 determines that oxygen supplementation is required, then, at step 608, the method 600 determines whether the FIO2 setting is achievable using oxygen supplementation only from POC flow between a minimum POC flow (Qpoc, min) and a maximum POC flow (Qpoc, max), without use of HPO2 flow. If so, then, at step 610, POC flow only is used for oxygen supplementation.

At step 608, if the method 600 determines that the FIO2 setting is not achievable using oxygen supplementation only from POC flow between Qpoc, min and Qpoc, max, then, at step 612, the method 600 determines whether using the minimum POC flow (Qpoc, min) would require overshooting the FIO2 setting. If so, then, at step 614, HPO2 flow only is used for oxygen supplementation (this is consistent with one aspect of operation of a Type A1 system, as described in detail with regard to later figures).

At step 612, if the method 600 determines that using the minimum POC flow (Qpoc, min) would not require overshooting the FIO2 setting, then, at step 616, the method 600 determines whether the FIO2 setting is achievable using maximum flow from the POC (Qpoc, max) plus flow from the HPO2 source. If so, then, at step 618, the maximum flow from the POC plus flow from the HPO2 source is used for oxygen supplementation.

If, at step 616, the method 600 determines that the FIO2 setting is not achievable using maximum flow from the POC (Qpoc, max) plus flow from the HPO2 source, then, at step 620, POC flow and HPO2 flow are used for oxygen supplementation, with a preference for using POC flow.

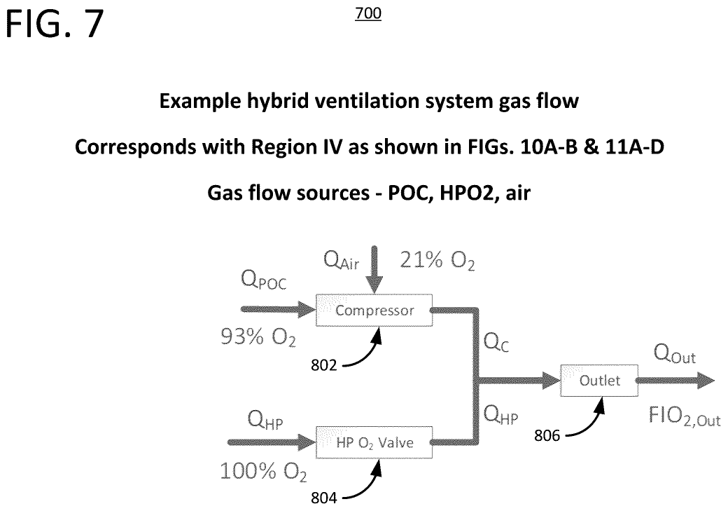

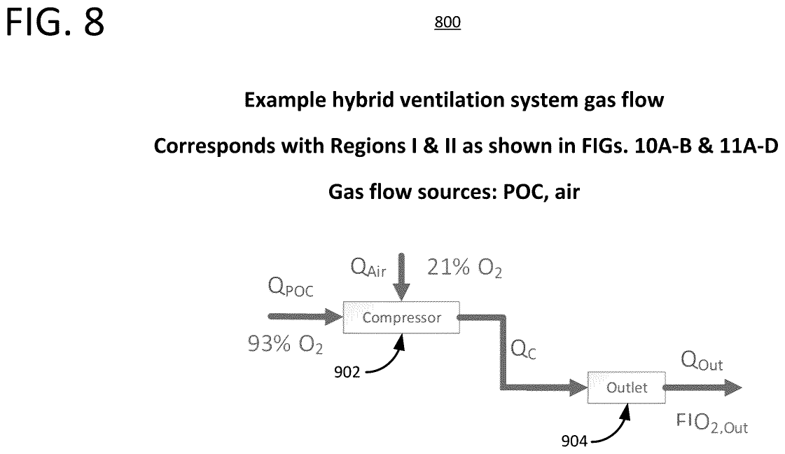

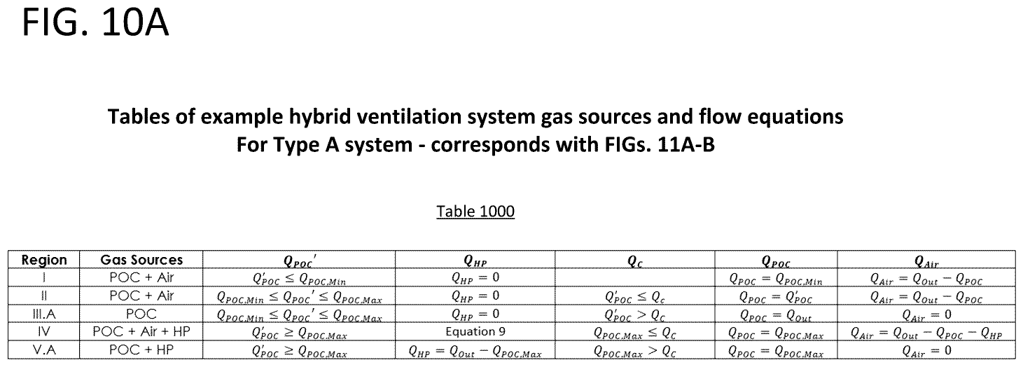

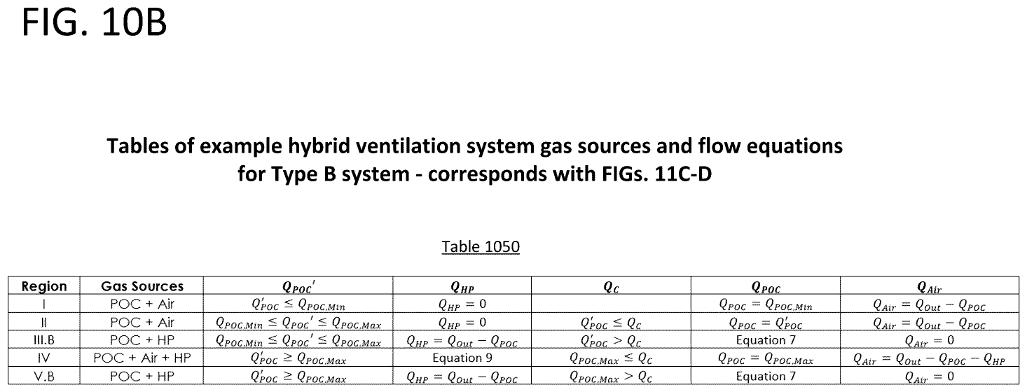

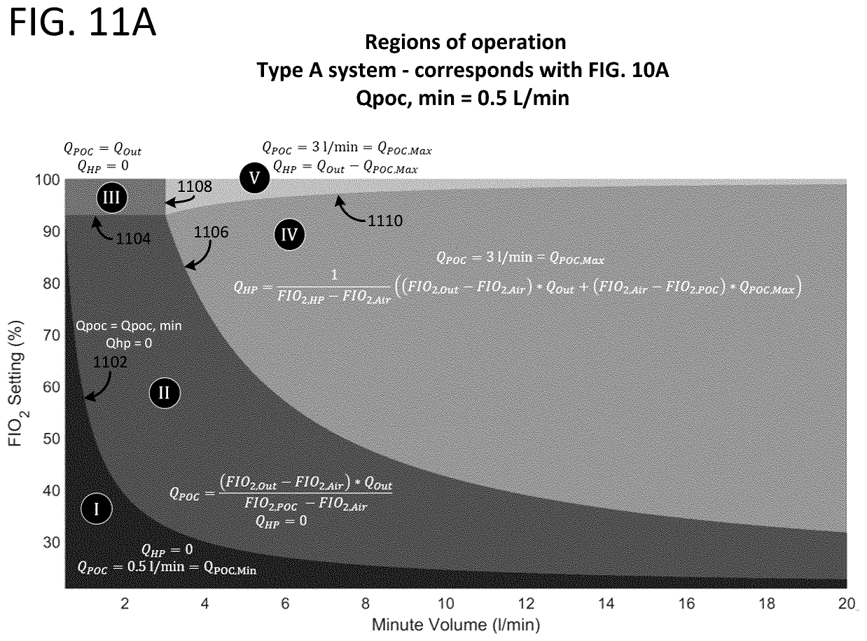

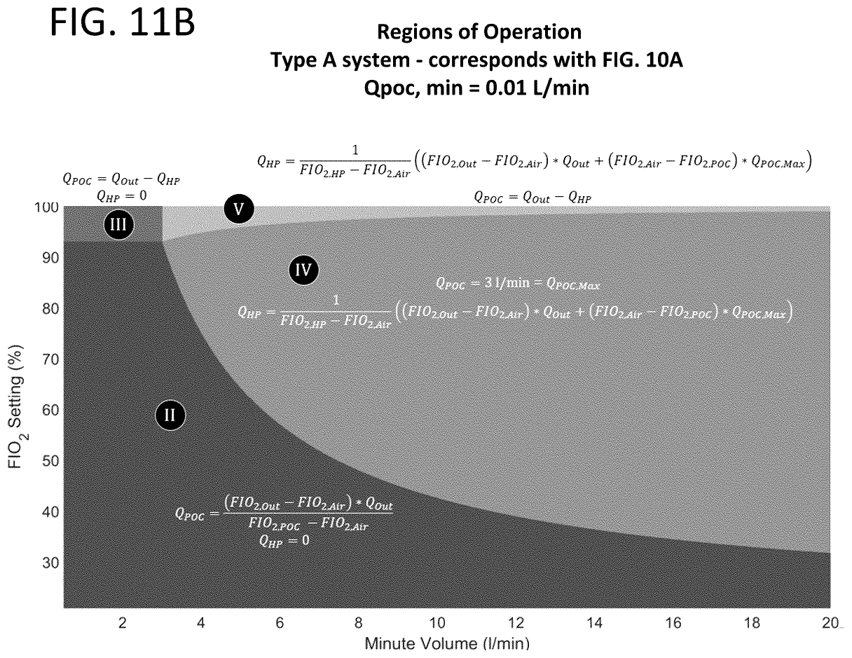

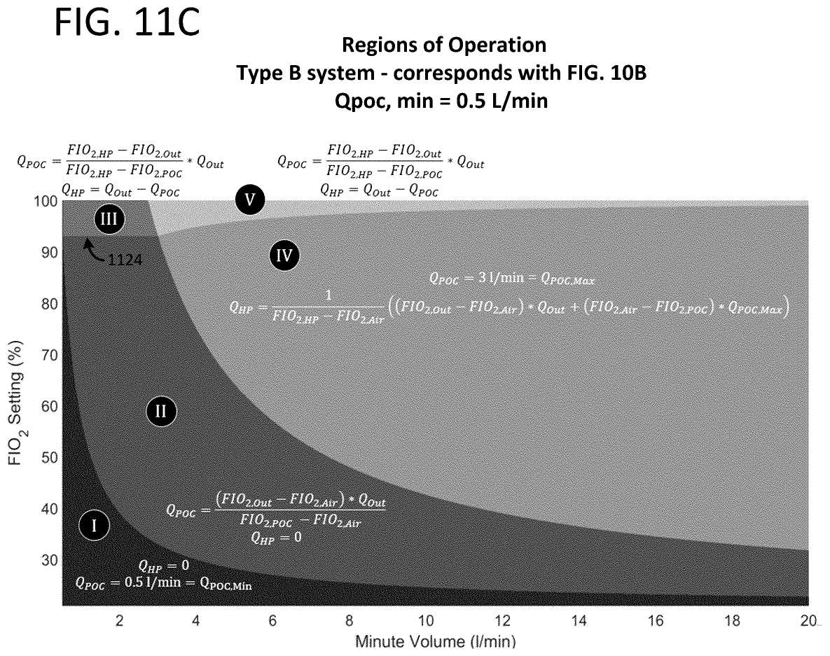

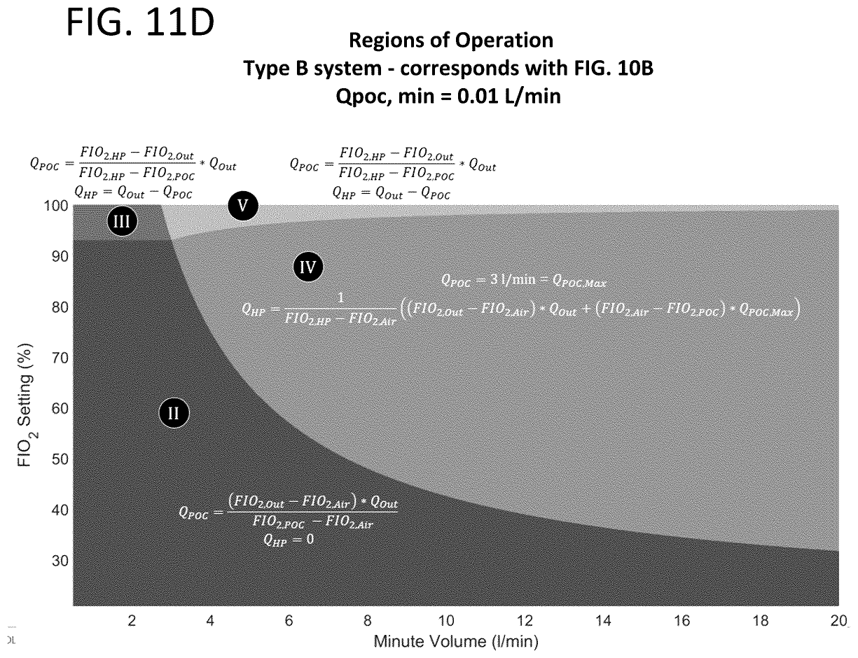

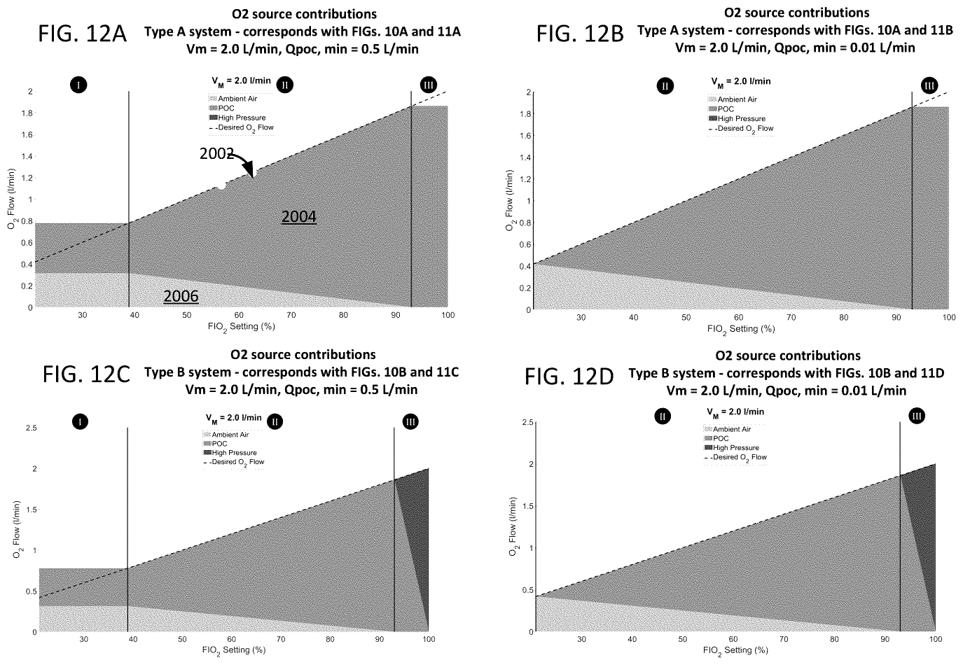

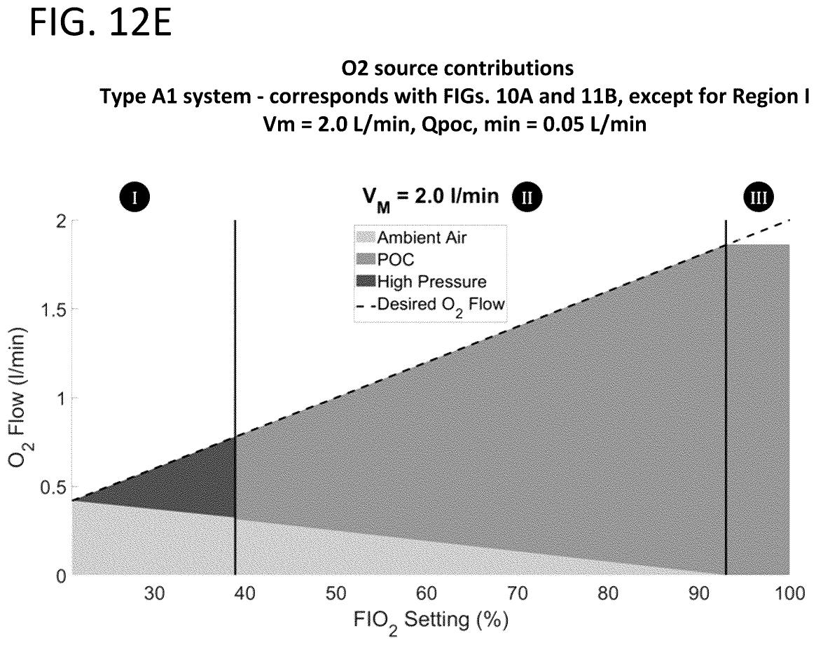

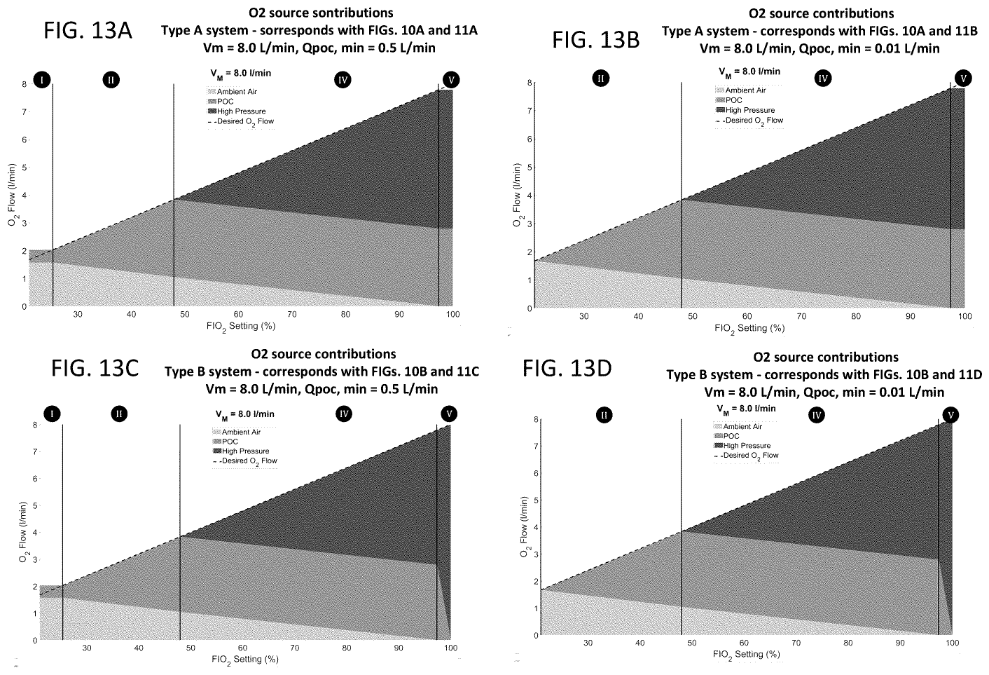

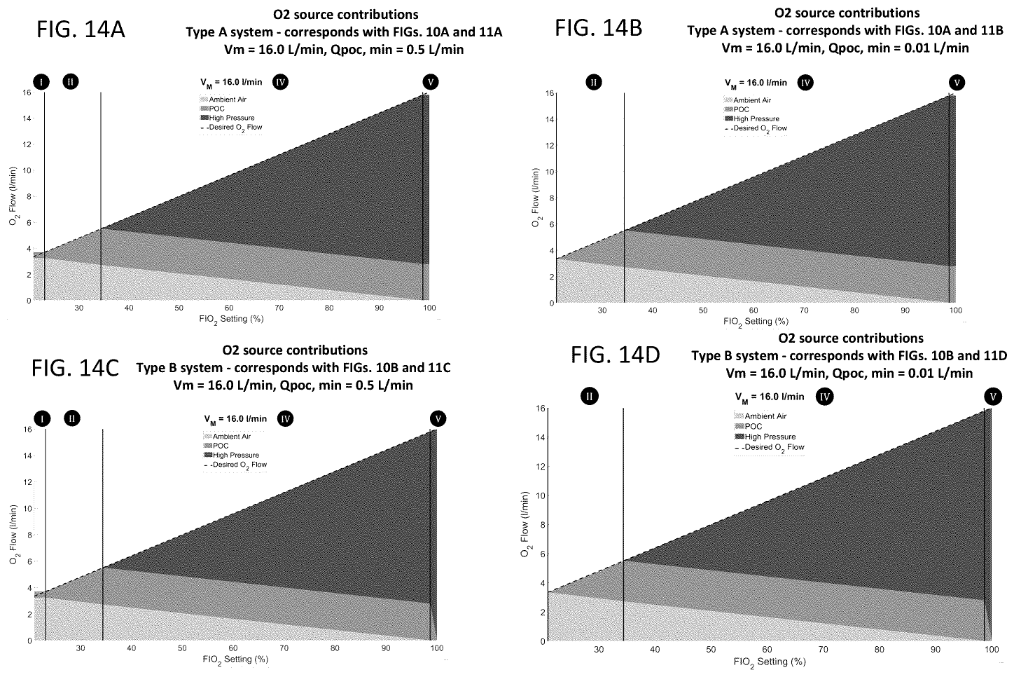

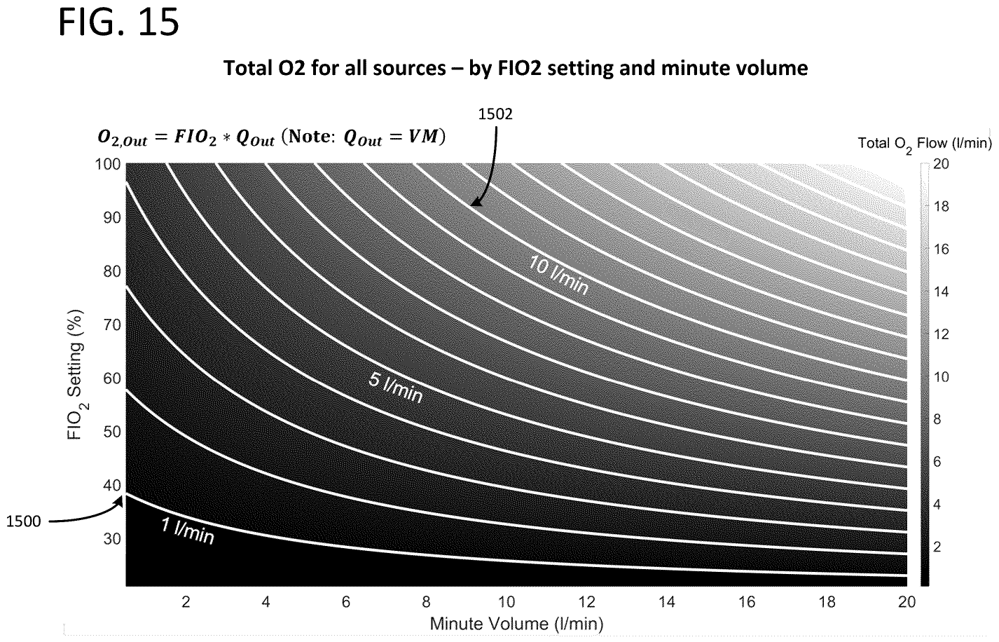

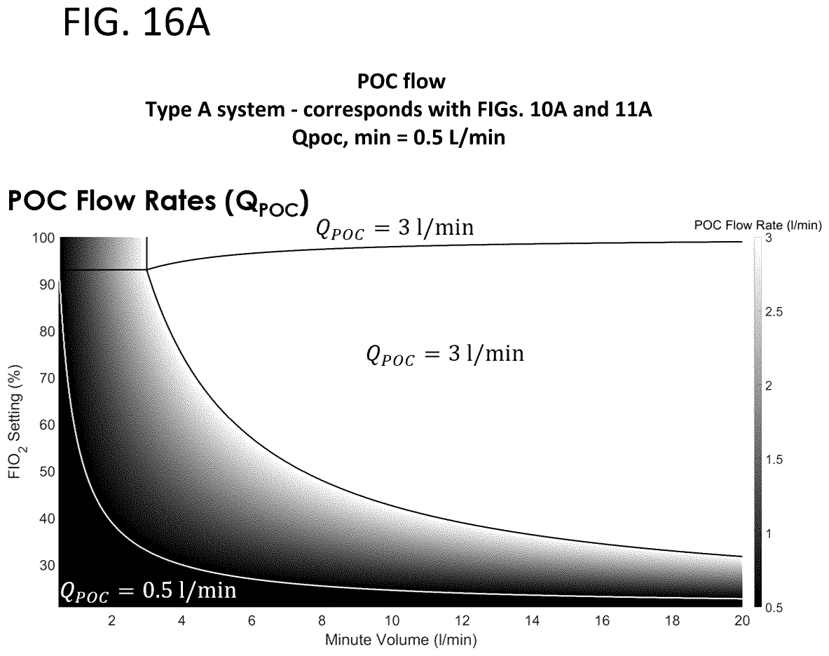

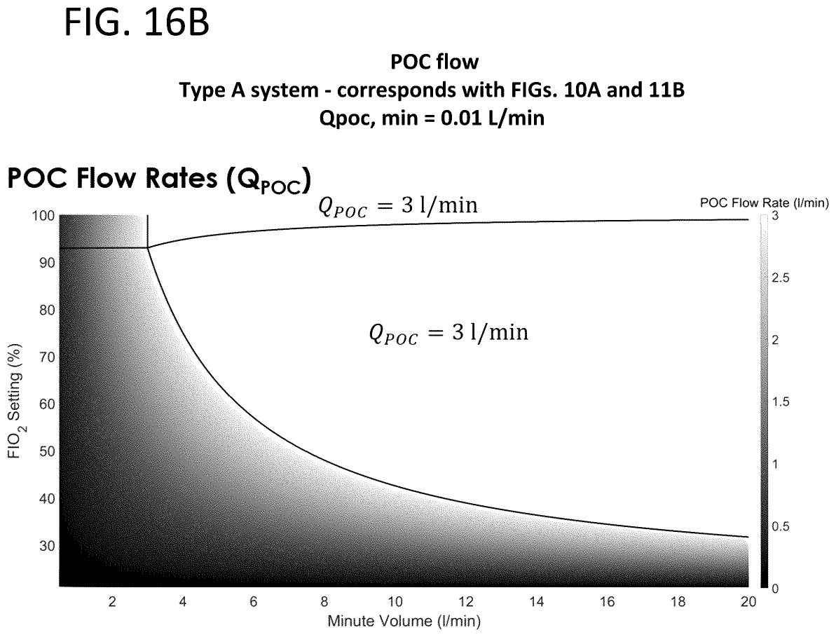

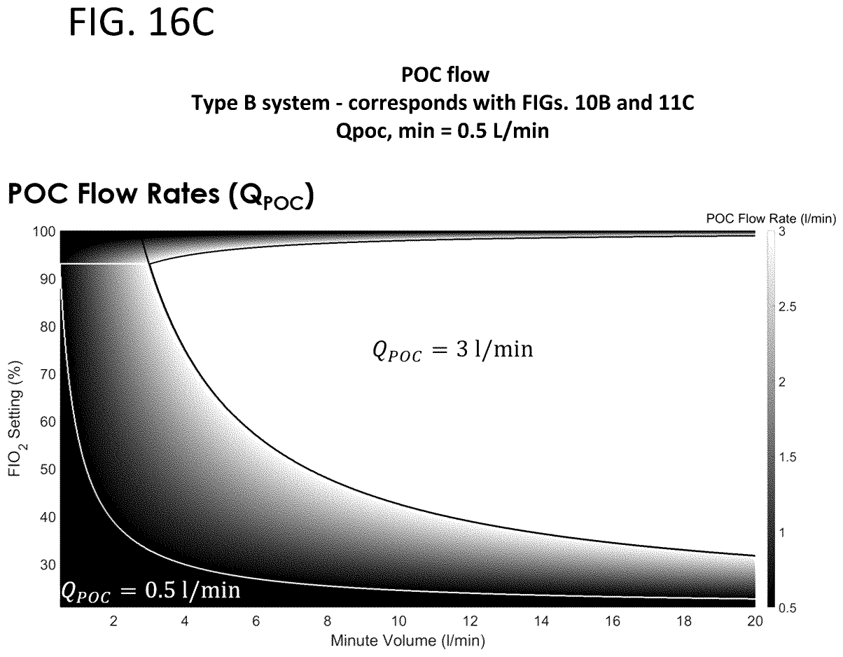

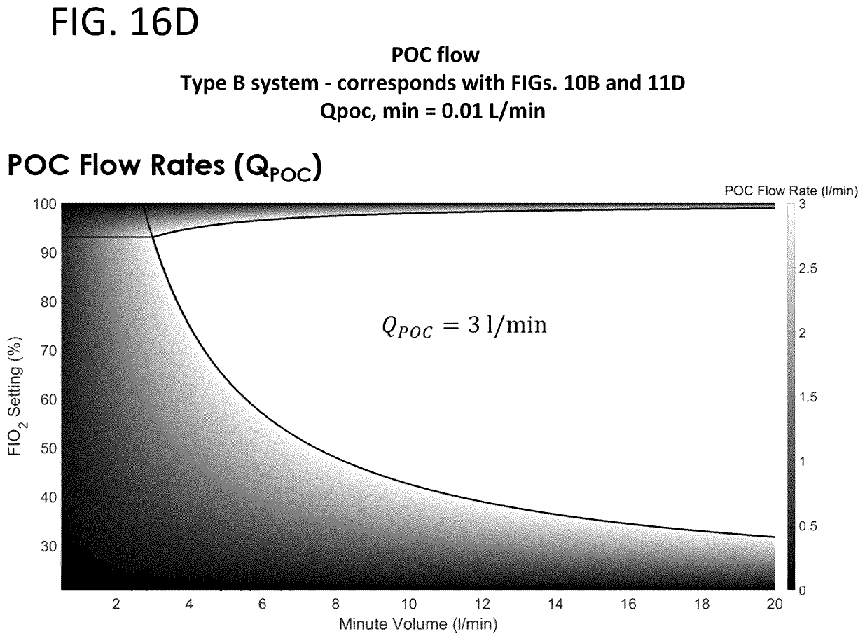

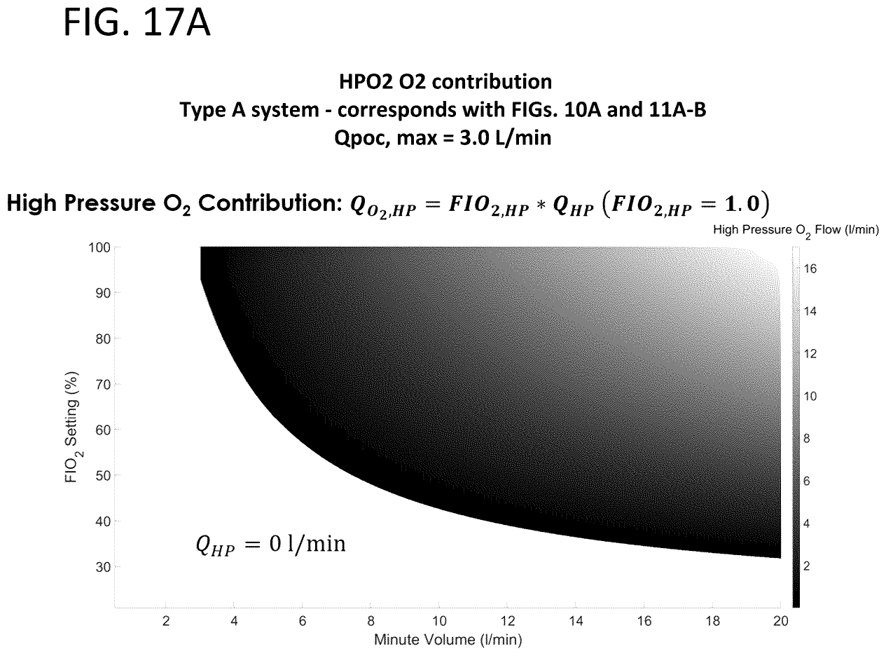

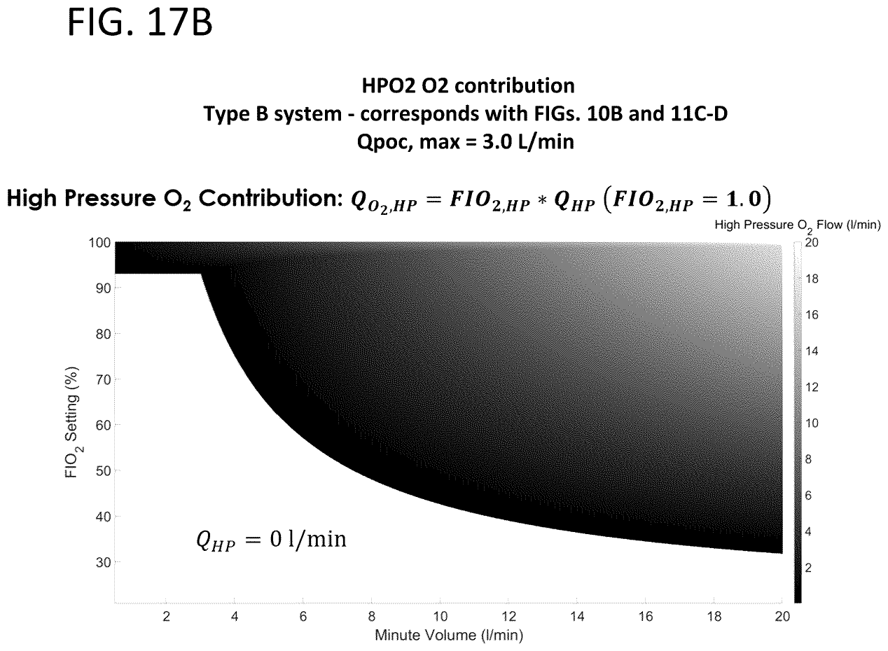

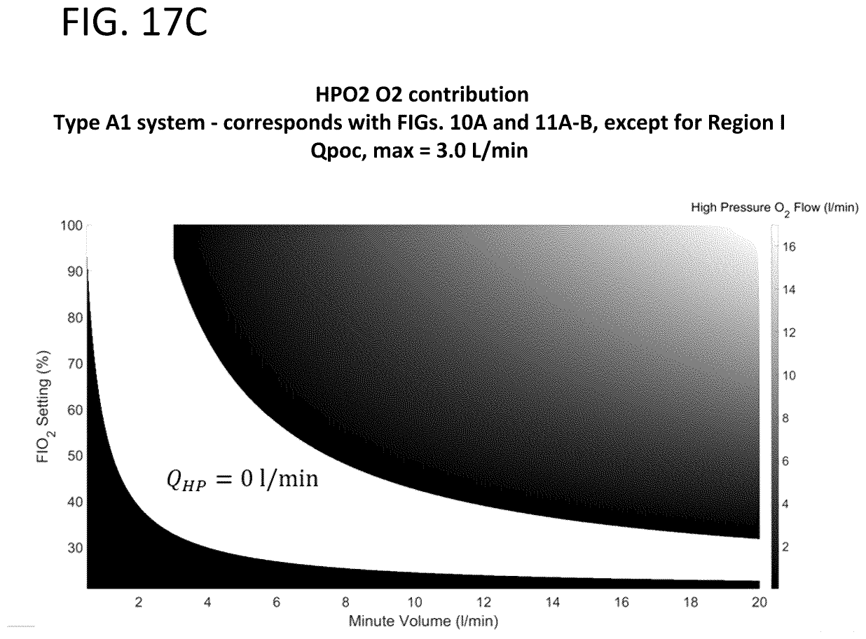

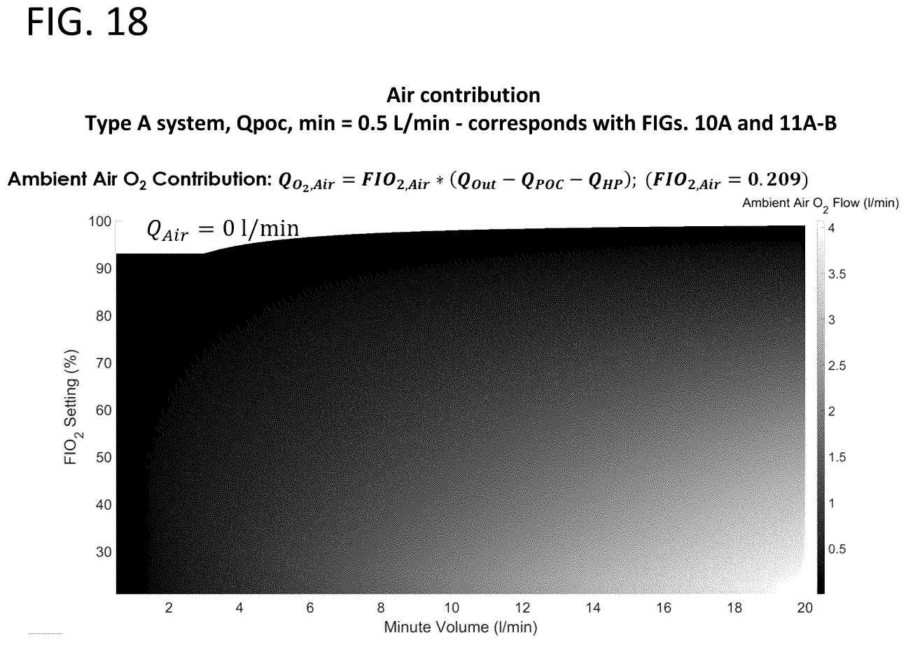

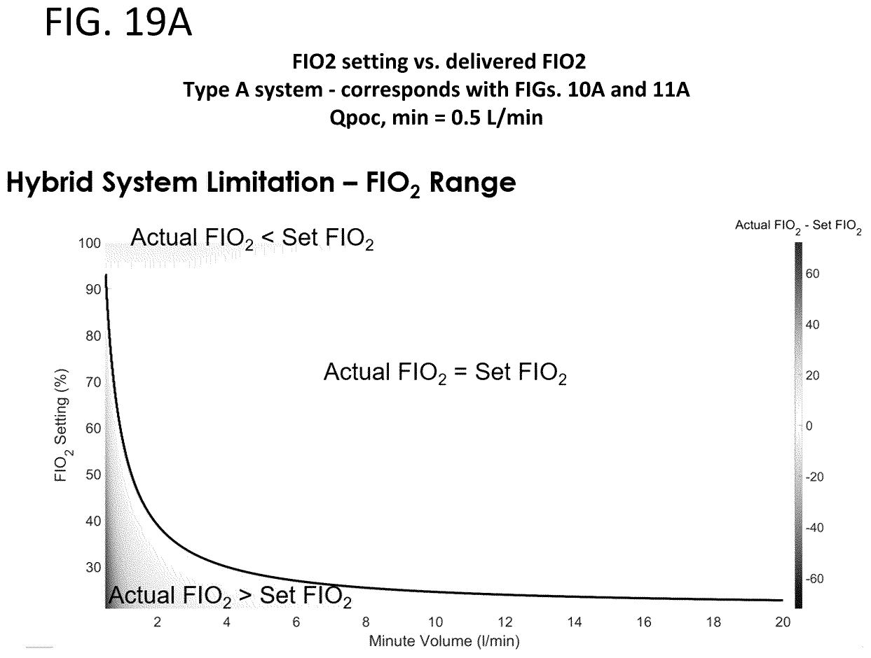

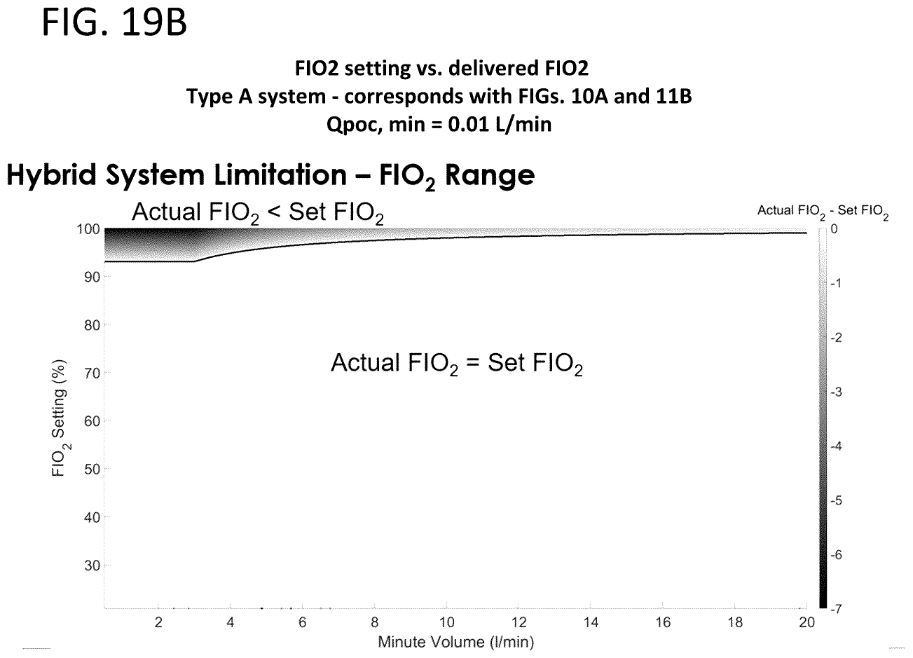

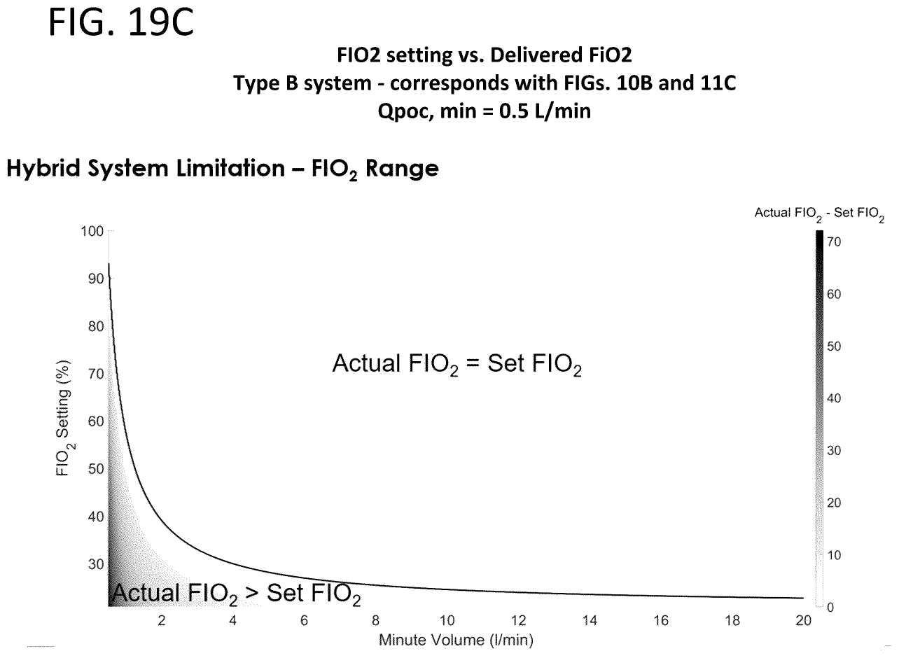

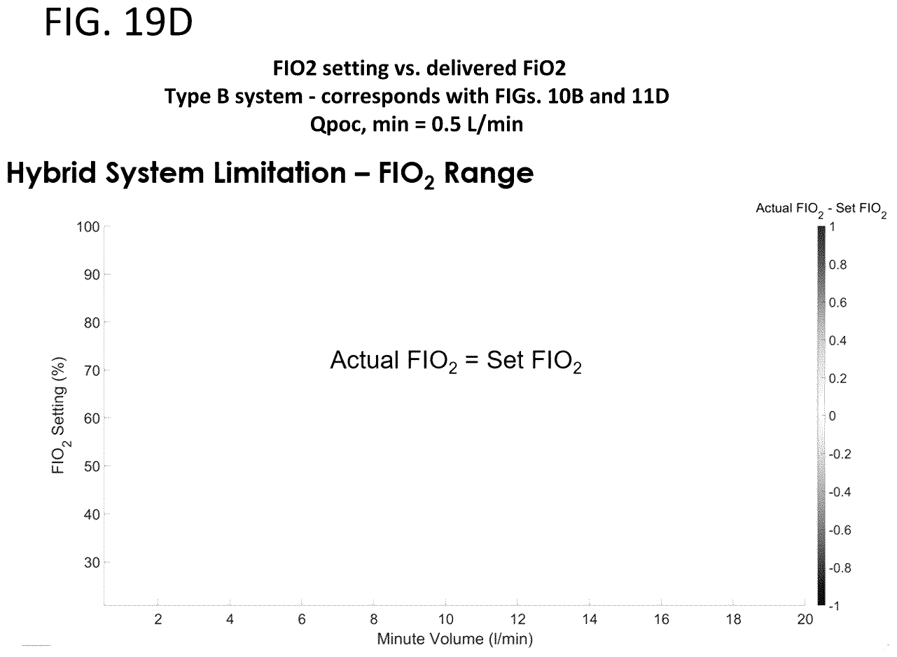

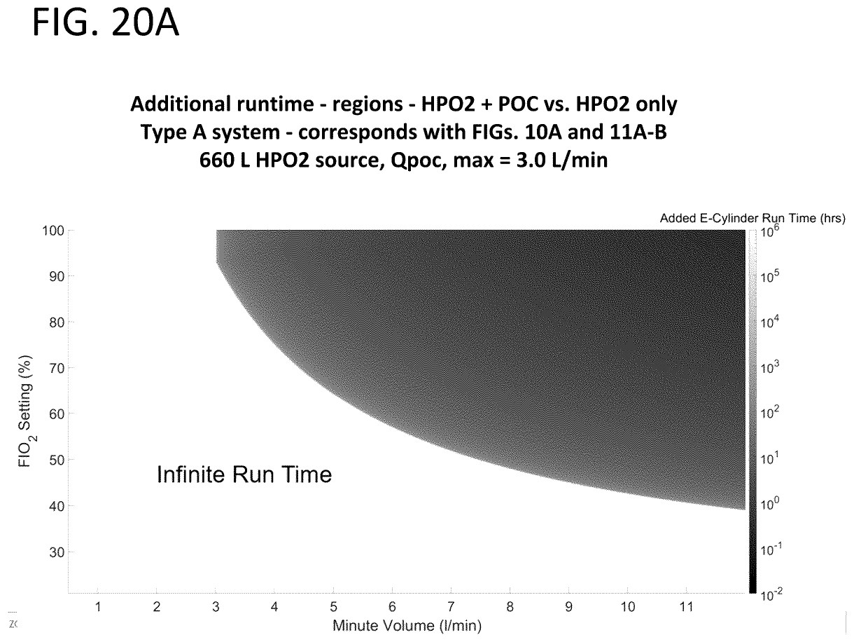

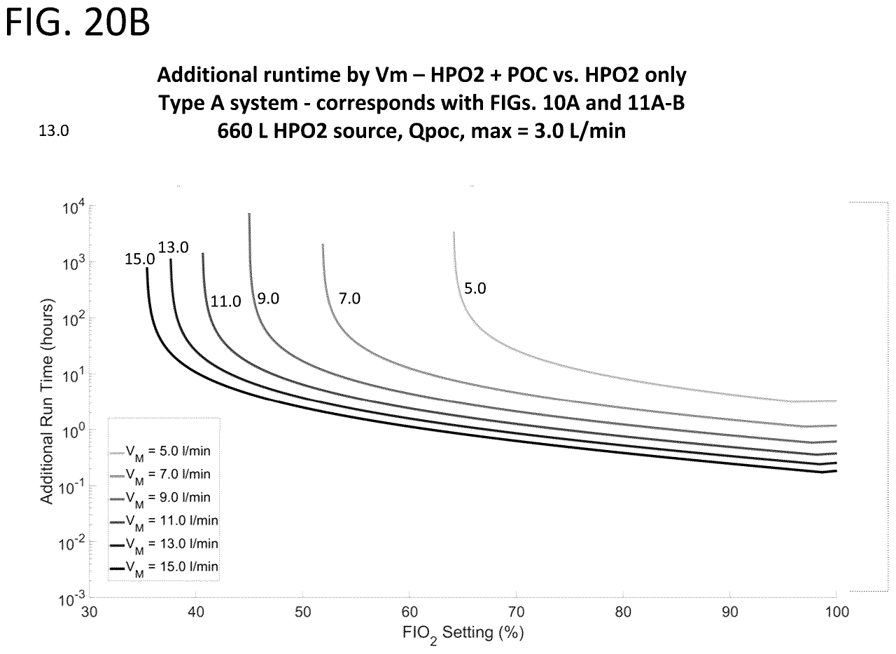

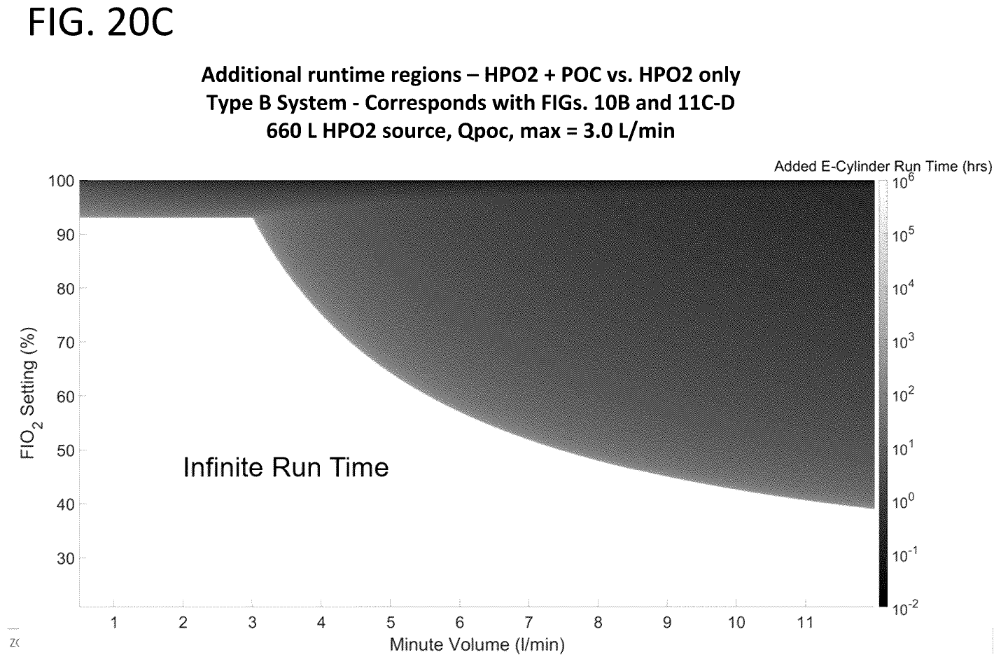

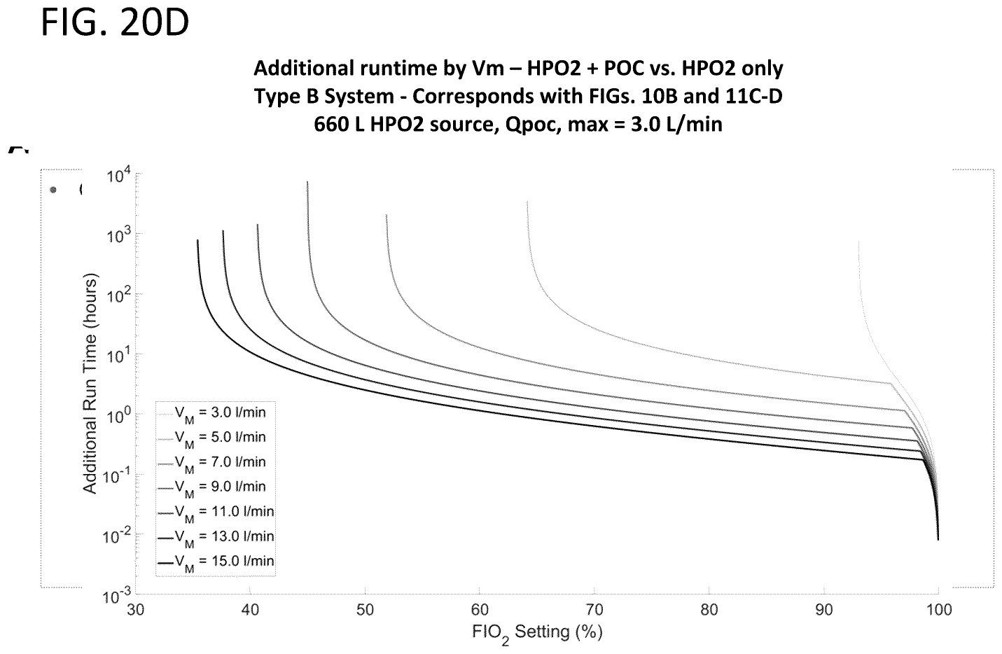

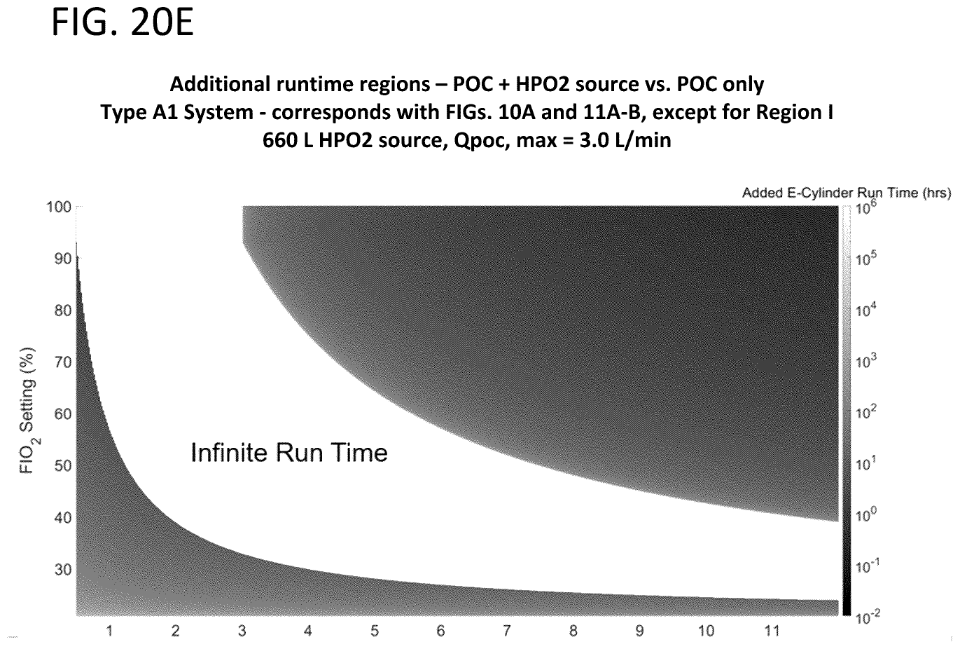

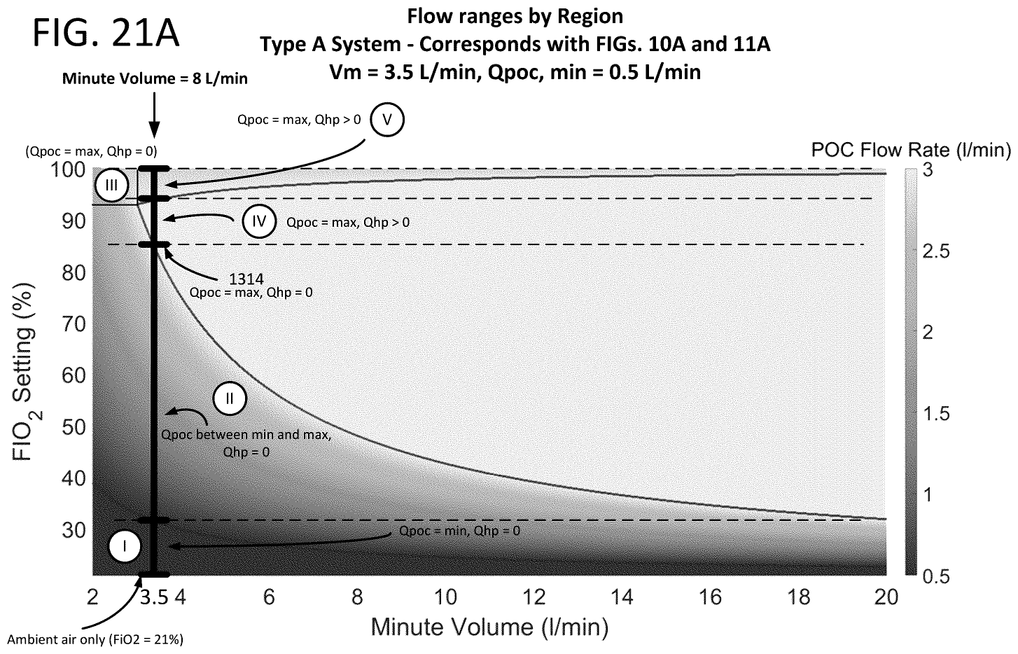

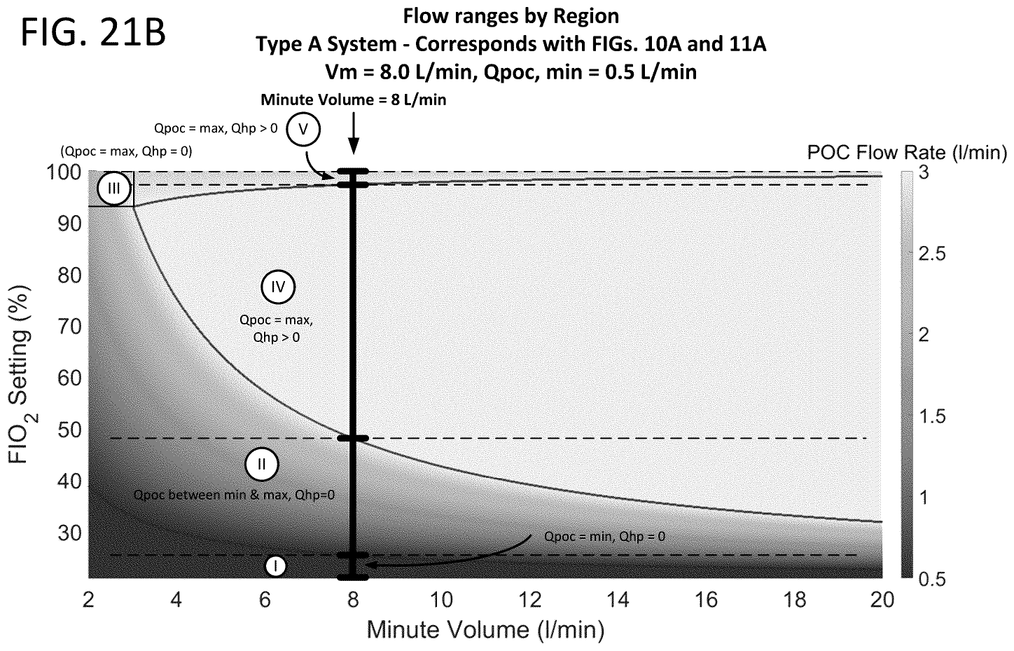

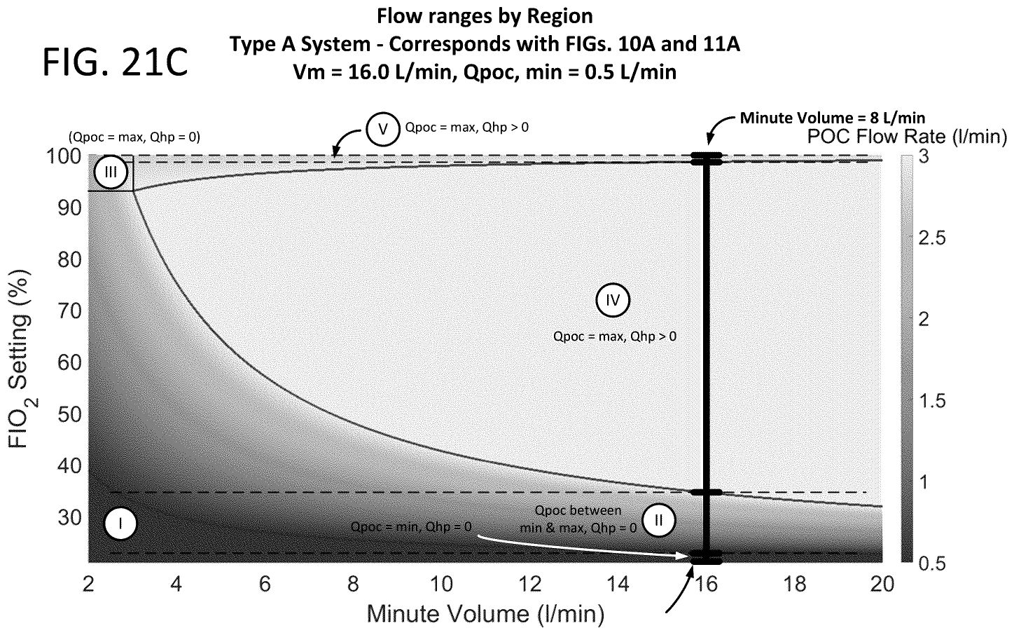

FIGS. 7-8 are block diagrams 700, 800 illustrating examples of contributing sources of gas flow in a hybrid ventilation system. In FIG. 7, in addition to the use of ambient air, oxygen supplementation is provided using the maximum POC flow (Qpoc, max) as well as flow from a HPO2 source, such as a HPO2 tank. FIG. 7 corresponds to Region IV as depicted in FIGS. 10A-10B and 11A-D.

As depicted, a compressor 802, of a portable ventilator of a hybrid ventilation system, inputs the flow from the POC (Qpoc), at an oxygen concentration of, e.g., 93%, and flow of ambient air (Qair) having an oxygen concentration of approximately 21% at sea level (or, to a more precise approximation, 20.9%), which combination of gases is output from the compressor 802 as Qc. Additionally, flow from the HPO2 tank is input to the portable ventilator via HPO2 valve 804 and is output as Qhp. Qc and Qhp combine to form the gas flow output through an outlet of the portable ventilator, as Qout (which includes Qpoc, max, Qair and Qhp), to the patient circuit and the patient. Qout has an FIO2 of FIO2out. In some embodiments, a hybrid FIO2 control of a controller of the portable ventilator (or elsewhere) may control the flow of gases to achieve a desired FIO2 for Qout, which provides the gas flow out of the portable ventilator and to the patient circuit and the patient.

In FIG. 8, for oxygen supplementation, maximum flow from the POC (Qpoc, max) only is used, without any flow from the HPO2 tank (Qhp). As shown, the compressor 902, of a portable ventilator of a hybrid ventilation system, inputs a combination of Qpoc and Qair, which combination of gases is output from the compressor as Qc. Qc forms the gas flow output through an outlet of the portable ventilator, as Qout (including only Qpoc and Qair) to the patient circuit and the patient.

FIG. 9 is a table 900 describing parameters used in some embodiments and examples herein. FIO2out is the FIO2 of gas output from the ventilator for delivery to the patient, which may range from, e.g., 21% (or 20.9%), the approximate oxygen concentration of ambient air at sea level, to 100% (all oxygen). In various embodiments, Qout may be set by the user or may be determined by, e.g., the controller of the portable ventilator without user input, such as may include use of FIO2 CLC, as described herein.

Qout, which may range from, e.g., 0-70 L/min, is the flow rate of gas delivered to the patient per minute, which is equal to the minute volume (Vm). In various embodiments, Qout may be set by the user or be determined by, e.g., the controller of the portable ventilator, and may also be measured. Qout can include flows associated with spontaneous breathing efforts by the patient.

Q′poc, which may range from, e.g., 0-100 L/min, is the flow rate from the POC to achieve a desired FIO2out, assuming no limitations in minimum or maximum POC flow rates. This is an intermediate, theoretical parameter, since, in fact, the POC may have a specified maximum flow rate (e.g., 3 L/min) and may have a specified minimum flow rate (e.g., 0.5 L/min, 0.01 L/min or 0 L/min). This parameter is used in equation derivations as described in some embodiments herein. In some embodiments, however, Q′poc is not utilized.

Qpoc, which may range from, e.g., 0.5-3.0 L/min, is the flow rate from the POC. In some embodiments, Qpoc may be set by the controller of the portable ventilator and may be measured by one or more flow sensors within the POC.

Qair, which may range from, e.g., 0-100 L/min, is the flow rate of ambient air drawn in by the compressor of the ventilator, and included in output gas delivered to the patient. In some embodiments, Qair may not be determined explicitly (such as by the controller of the portable ventilator), but may be effectively determined by other flow rates. For example, in some embodiments, Qair may be used to add as needed to the flow rates of other gases to achieve a desired combined or composite total flow rate. However, in some embodiments, Qair may be explicitly determined.

Qc, which may range from, e.g., 0-100 L/min, is the flow rate of gas through the compressor, where, in some embodiments, Qc=Qpoc+Qair.

Qhp, which may range from, e.g., 0-100 L/min, is the flow from the HPO2 source, and may be set by the controller of the ventilator.

FIO2poc, which may be, e.g., 93%, or, e.g., between 90-96%, is the oxygen concentration of gas output by the POC, which may be measured.

FIO2air is the oxygen concentration of ambient air from the environment, which may be an assumed value of 21% (or 20.9%) at sea level.

FIO2 hp is the oxygen concentration of gas output by the HPO2 source, which may be, e.g., 100%.

FIO2′, which may be between, e.g., 21-100%, is an internal portable ventilator value that yields the desired Qhp and Qout. In some embodiments, however, FIO2′ is not included and not utilized by the ventilator.

Qpoc, min which may be, e.g., 0.5 L/min, 0.01 L/min or 0 L/min, is the minimum flow rate of gas output by the POC, which may be a device limit associated with the particular POC.

Qpoc, max, which may be, e.g., 3.0 L/min, is the maximum flow rate of gas output by the POC, which may be a device limit associated with the particular POC.

In FIGS. 10A-B, various parameters, including parameters described with reference to table 900 of FIG. 9, are used in characterizing aspects of regions of operation of example hybrid ventilation systems. FIGS. 11A-D provide plots illustrating the regions of operation for example hybrid ventilation systems. FIGS. 12A-D-21A-C then provide plots illustrating aspects of operation of example hybrid ventilation systems, including, among others, those characterized in table 900 of FIG. 9, and in the plots of FIGS. 11A-D.

In the following, which includes equation derivations, reference is made to differences in operation between different example types of hybrid ventilation systems. These include example Type A and Type B systems, which differ with respect to particular regions of operation including high FIO2 settings defined as Regions III and IV, below. Additionally, an example Type A1 system is described, which varies from the Type A system with respect to operation in a region of operation including low FIO2 settings and low minute volumes defined as Region I, below.

Qout, as described with reference to FIG. 9, may be made up of the flow rate of gas from the compressor, Qc (if any), plus the flow rate of gas from the HPO2 source (if any), Qhp. As such:

Qout=Qc+Qhp (Equation 1)

In some embodiments, a 3 L oxygen reservoir bag is attached to an inlet to the compressor of the ventilator, to provide an interface to the ventilator for the POC (however, in various embodiments, reservoir bags of various volumes can be used). Flow from the POC (at a flow rate of Qpoc) may fill the reservoir bag during the expiratory phase of a ventilation breath (and when Qc is less than Qpoc). The flow of gas through (drawn in and expelled out) of the compressor may be made up of POC flow (if any) and air flow (if any), such that:

Qc=Qpoc+Qair (Equation 2)

Combining equations 1 and 2 yields:

Qout=Qpoc+Qair+Qhp (Equation 3)

As shown in FIG. 7, the flow of gas out of the ventilator and to the patient may be made up of the gas flow from the POC plus the gas flow from the compressor, and the gas flow from the compressor may include both flow of air and flow from the HPO2 source. This is expressed by Equation 3, above.

For any gas flow (or flow rate), the oxygen gas flow (QO2) portion is the total flow multiplied by the fraction of oxygen in the total flow. As such, oxygen flow rate may be calculated as:

QO2=FIO2*Q (Equation 4)

The fraction of oxygen in the HPO2 source may be 100% and the fraction of oxygen in ambient air is typically approximated as 21%, or may be approximated more precisely as 20.9%, for example. The oxygen concentration of output gas from the POC may be measured by an internal sensor of the POC and may be equal to, e.g., 93% plus or minus 3% (or more or less), in accordance with the specifications of the POC. Combining equations above, the total oxygen flow from the ventilator to the patient (QO2out) may be expressed as follows:

Qo2out=FIO2out*Qout=FIO2hp*Qhp+FIO2poc*Qpoc+FIO2air*Qair (Equation 5)

Type A and Type B hybrid ventilation systems differ with regard to operation in particular regions of operation, as described below, requiring achieving high FIO2 settings, such as may include FIO2 settings greater than FIO2poc (e.g., 93%). The Type A system may have an advantage, potentially among others, of operating such that, for such high FIO2 settings, flow from the HPO2 source is less, relative to that of the Type B system, and by potentially requiring a less complex system. This operation of the Type A system, in connection with these particular regions, may cause use of less HPO2 flow relative to that of the Type B system. However, the Type A system may have a disadvantage, potentially among others, relative to the Type B system, in that, to a degree, it underdelivers oxygen and undershoots the FIO2 setting in these regions.

The Type B system may have an advantage, potentially among others, that it can deliver oxygen without underdelivery, and it can achieve the FIO2 setting without undershooting it, even for, e.g., FIO2 settings higher than FIO2poc, and at all minute volumes. However, relative to the Type A system, it may have disadvantages, potentially among others, of using more flow from the HPO2 source and requiring greater system complexity, such as may relate to requiring more, or more rapid, POC flow ramp up and ramp down.

As such, in some embodiments, for example, the determination of whether to use a Type A or a Type B system, may depend on a balancing, comparison or weighing of advantages and disadvantages, including those described above. Particularly, for example, with regard to the Type A system relative to the Type B system, any potential negative or sub-optimal effects, such as may relate to patient treatment, or the undershooting, to a degree, of the FIO2 setting, may need to be weighed against advantages, such as use of less HPO2 flow (which can, for example, extend the runtime of the system) and potentially reduced system complexity.

In both Type A and Type B systems, in some embodiments, to determine flow rates of gases, the system (e.g., a controller of the ventilator) first calculates the flow rate from the POC that would be necessary to achieve the desired FIO2out, assuming (theoretically) that the POC was the only source of oxygen (so Qhp=0) and had no minimum or maximum flow rates, which calculated flow rate is called Q′poc. As such, using Equation 5 and substituting Qhp=0 yields:

Q′poc=[(FIO2out−FIO2air)/(FIO2poc−FIO2air)]*Qout (Equation 6)

The flow from the HPO2 source is next calculated. For both Type A and Type B systems, in Region I of operation of the system (as shown, e.g., in FIGS. 11A-D), which includes low FIO2 settings and low minute volumes, Q′poc<Qpoc, min. For example, if Qpoc, min=0.5 L/min, then Region I occurs when Q′poc<0.5 L/min. With respect to operation in Region I, two different variations are described in connection with Type A and Type A1 systems.

In the Type A system (as shown, e.g., in FIGS. 11A-B), in Region I, Qpoc is set to Qpoc, min and no flow from the HPO2 source is utilized (Qhp=0). This variation has a disadvantage, relative to a Type A1 system, in that, for POC with a minimum flow rate, to a degree, it overdelivers oxygen and, to a degree, it overshoots the FIO2 setting, in Region I. However, relative to the Type A1 system, the Type A system may have an advantage of being simpler and less difficult to implement, since it may require less frequent and rapid ramp up and ramp down in POC output or turn on and turn off of the POC. Additionally, the Type A system has an advantage of using less HPO2 flow and supply, relative to the Type A1 system. Furthermore, any negative or non-optimal treatment impact of the overshooting, to a degree, of the FIO2 setting may, in some cases, be negligible or minimal.

In the Type A1 system, in Region I, as described below, Qpoc is set to 0 (so there is no flow from the POC delivered to the patient), and flow from the HPO2 source (Qhp) is used for supplemental oxygen. For example, in various embodiments, to set Qpoc=0, the POC may be switched into standby mode or temporarily turned off. Alternatively, it may be left in regular mode or left on, but its flow may be temporarily diverted away from the patient. For a POC with a minimum flow rate, the Type A1 system may have an advantage, relative to the Type A system, in Region I, of being able to provide oxygen delivery without overdelivery and achieve the FIO2 setting without overshooting it. However, the Type A1 system may have a disadvantage, relative to the Type A system, in Region I, of using more HPO2 flow. Furthermore, the Type A1 system may have a disadvantage of being more complex or difficult to implement than the Type A system, such as with regard to switching the POC into and out of standby mode, or on and off, or by requiring diversion of POC flow in some instances. Additionally, the Type A1 system may have a disadvantage, relative to the Type A system, of introducing difficulty or sub-optimal operation in connection with the time that may be required to ramp up output gas oxygen concentration or gas flow rate from the POC. For example, some POCs require a waiting or “warm-up” period before they can deliver the maximum flow rate or full oxygen concentration (e.g., 93%).

As such, in some embodiments, for example, the determination of whether to use a Type A or a Type A1 system may depend on a weighing of advantages and disadvantages, as described above. Particularly, for example, with regard to the Type A system relative to the Type A1 system, any potential negative or sub-optimal effects, such as may relate to patient treatment, of the overshooting of the FIO2 setting, may need to be weighed against advantages such as use of less HPO2 source flow (which can extend the runtime of the system) and reduced system complexity.

It is noted that, while the Type A1 system is described, relative to the Type A system, in some embodiments, a similar variation may be introduced in other example hybrid ventilation systems. For example, a Type B1 system could be provided or used that incorporates the same variation to the Type B system as the Type A1 system does to the Type A system.

Furthermore, it is noted that, in some embodiments, an example hybrid ventilation system may be provided or used that is capable of operating, e.g., at different times or in different circumstances, in different ways that may correspond to any of multiple different system types, such as, e.g., based on a user selection or an automatic, algorithm-based determination. For example, a hybrid ventilation system may be capable of operating as a Type A or Type B system, a Type A or A1 system, a Type B or B1 system, or any combination of the foregoing, among others. Whether such a system is desirable or preferable may, e.g., depend on a balance or comparison of any advantage of increased system operation flexibility relative to any disadvantage of increased system complexity.

In Region II (as shown, e.g., in FIGS. 11A-D), Q′poc is between Qpoc, min and Qpoc, max, and Q′poc is less than or equal to Qout. In region II, Qpoc is set to Q′poc and Qhp is set to 0.

In Region III (as shown, e.g., in FIGS. 11A-D), where Q′poc is between Qpoc, min and Qpoc, max, and when Q′poc is greater than Qout, the Type A and Type B systems differ in operation.

In the Type A system (as shown, e.g., in FIGS. 11A-B), Qpoc is set to Qout and both Qhp and Qair are set to zero. In table 1000 of FIG. 10A, this corresponds to Region III.A.

However, in the Type B system (as shown, e.g., in FIGS. 11A-B), Qair is set to 0 and Qpoc is determined based on Equation 5, solved for Qpoc, as follows:

Qpoc=[(FIO2hp−FIO2out)/(FIO2hp−FIO2poc)]*Qout (Equation 7)

Furthermore, Qhp is included and is determined as Qhp=Qout−Qpoc. In table 1050 of FIG. 10B, this corresponds to Region III.B.

Furthermore, the Type A and Type B systems differ in operation with regard to Region V (as shown, e.g., in FIGS. 11A-D) where Q′poc is greater than or equal to Qpoc, max and Qpoc, max is greater than Qc.

In the Type A system, Qpoc is set to Qpoc, max and the remaining portion of Qout comes from Qhp, where Qhp=Qout−Qpoc, max. In table 1000 of FIG. 10A, this corresponds to Region V.A.

In the Type B system, Qpoc is determined using Equation 7, and the remaining portion of Qout comes from Qhp, where Qhp=Qout−Qpoc, max. In table 1050 of FIG. 10B, this corresponds to Region V.B.

For Region IV (as shown, e.g., in FIGS. 11A-B), both the Type A and Type B systems operate similarly. Qhp is used in achieving the desired FIO2 setting.

For Region IV, Qhp may be determined in the following way. First, equation 3 is solved for Qair as follows:

Qair=Qout−Qhp−Qpoc (Equation 8)

Next, Equations 5 and 8 may be combined and rearranged to obtain the following:

Qhp=[1/(FIO2hp−FIO2air)]*[(FIO2out−FIO2air)*Qout+(FIO2air−FIO2poc)*Qpoc,max] (Equation 9)

In some ventilation systems that include a HPO2 source but do not include a POC, flow rate from the HPO2 source may not be directly calculated, but instead may be determined based on a FIO2out and minute volume. In such systems, in normal operation without a POC, it is assumed that ambient air is drawn into the intake to the compressor. As such:

QoutFIO2′=FIO2air*Qc+FIO2hp*Qhp (Equation 10)

Equations 1 and 10 can then be used to solve for FIO2′, as a function of Qhp and Qout, as follows:

FIO2′=FIO2air+(FIO2hp−FIO2air)*(Qhp/Qout) (Equation 11)

Further substitutions can be used to solve for FIO2′, as a function of Qpoc and Qout, as follows:

FIO2′=FIO2out−(FIO2poc−FIO2air)*(Qpoc/Qout) (Equation 12)

FIGS. 10A-B are tables 1000, 1050 illustrating examples of gas sources and aspects relating to flow rates associated with regions of operation of example Type A and Type B hybrid ventilation systems. Particularly, table 1000 of FIG. 10A relates to a Type A system, and table 1050 relates to a Type B system. As described above, these systems operate similarly in Regions I, II and IV, but differently in Regions III and V. As such, in table 1000, Regions III and IV are represented, respectively, as Regions III.A and V.A, and, in table 1050, Regions II and IV are represented, respectively, as Regions II.B and V.B.

Tables 1000, 1050 are described further below, after an introductory description of FIGS. 11A-D as follows, which show Regions of operation referred to in tables 1000 and 1050. FIGS. 11A-D show regions of operation in connection with gas flows for example Type A and Type B systems, for a range of FIO2 settings (along the y axes) and over a range of minute volumes (along the x axes). More particularly, FIGS. 11A-B illustrate plots relating to regions of operation for example Type A systems (with minimum POC flow rates of 0.5 L/min and 0.01 L/min, respectively), and therefore correspond to table 1000 of FIG. 10A. Furthermore, FIGS. 11C-D illustrate plots relating to regions of operation for example Type B systems (with minimum POC flow rates of 0.5 L/min and 0.01 L/min, respectively), and therefore correspond to table 1050 of FIG. 10B.

Returning to tables 1000, 1050, for Region I, gas sources for gas delivered to the patient include POC flow and ambient air, Q′poc is less than or equal to Qpoc, min, Qhp=0, Qpoc=Qpoc, min, and Qair=Qout−Qpoc. As such, in Region I, gas flow from the POC is used at the minimum POC flow (Qpoc, min), along with ambient air. Since table 1000 relates to a Type A system, as described above, for a POC with a minimum flow rate (or a minimum flow rate that is not approximated as zero L/min), the supplied oxygen and FIO2 of gas delivered to the patient overshoots, to a degree, the FIO2 settings in this region.

As can be seen in FIGS. 11A and 11C, Region I applies to low FIO2 settings and low minute volumes, where relatively low oxygen supplementation is needed to achieve the FIO2 settings, such that even the minimum POC flow rate of 0.5 L/min overshoots, to a degree, the FIO2 settings in this region. As to FIGS. 11B and 11D, since the minimum POC flow is 0.01 L/min, it is approximated, in these figures, that there is no minimum POC flow rate (and, in some embodiments, the minimum POC flow may be 0 L/min, or between 0 L/min and 0.01 L/min, or more than 0.01 L/min, for example). As such, it is never necessary to overshoot the FIO2 settings using a POC minimum flow rate. Therefore, Region I is absent in FIGS. 11B and 11D, and Region II of FIGS. 11B and 11D includes the region that is Region I in FIGS. 11A and 11C.

As shown in tables 1000 and 1050, for Region II, gas sources for gas delivered to the patient include POC flow and ambient air, Q′poc is between Qpoc, min and Qpoc, max, Qhp=0, Q′poc is less than or equal to Qc, Qpoc=Q′poc, and Qair=Qout−Qpoc. As can be seen in FIGS. 11A-D, Region II applies generally to ranges of minute volumes and FIO2 settings where oxygen supplementation from flow from the POC, between its minimum and maximum output flow rates, is sufficient to achieve the FIO2 setting, without any additional supplementation from the HPO2 source. In FIGS. 11B and 11D, since Qpoc, min is approximated to be zero, Region II extends even to low FIO2 settings and minute volumes.

In FIG. 11A, border 1102 is the border between Regions I and II. At this border 1102, POC flow is at its minimum (Qpoc=Qpoc, min) and no HPO2 flow is included (Qhp=0).

As shown in table 1000 of FIG. 10A, for a Type A system, for Region III of FIGS. 11A-B, the only gas source for gas delivered to the patient is POC flow, Q′poc is between Qpoc, min and Qpoc, max, Qhp=0, Q′poc is greater than Qc, Qpoc=Qout, and Qair=0. However, as shown in table 1050, for a Type B system, Region III of FIGS. 11C-D differs from that of FIGS. 11A-B in that gas sources include POC flow and HP gas flow, Qhp=Qout−Qpoc, and Qpoc is given by Equation 7, above. As described in detail previously, in this region of high FIO2 settings and low minute volumes, in a Type A system, as illustrated in FIGS. 11A-B, HPO2 flow is not used, and is therefore conserved, but this comes at the cost of undershooting, to a degree, the FIO2 setting in this limited region. However, in the Type B system, even in this region, the system can achieve the FIO2 setting without undershooting it, but this may come at the cost of using flow from the limited HPO2 source and greater system complexity.

In FIG. 11A (for an example Type A system), border 1104 represents the border between regions II and III. This border 1104 occurs at FIO2=93%, which, in this example, is equal to the oxygen percentage of flow from the POC. This border 1104 represents the threshold beyond which only POC flow is used, without any ambient air flow or HPO2 flow (which, as described above, undershoots the FIO2 setting).

In FIG. 11C (for an example Type B system), border 1124 represents the border between Regions II and III. This border 1124 represents the threshold beyond which POC flow and air flow is replaced with POC flow and HPO2 flow (which, as described above, achieves the FIO2 setting without undershooting it).

As shown in tables 1000 and 1050, for Region IV, gas sources include POC flow, ambient air and HPO2 source gas flow, Q′poc is equal to or greater than Qpoc, max, Qhp is given by Equation 9, Qpoc, max is less than or equal to Qc, Qpoc=Qpoc, max and Qair=Qout−Qpoc−Qhp. In this region, which covers a wide range of FIO2 settings and minute volumes, the FIO2 settings are achieved using maximum flow from the POC with additional flow from the HPO2 source as required.

In FIG. 11A (for an example Type A system), border 1106 represents the threshold between regions II and IV. This border 1106 represents the threshold beyond which POC flow and air flow is replaced by POC flow, air flow and HPO2 flow, where use of HPO2 flow allows achieving higher FIO2 settings.

Finally, as shown in table 1000, for a Type A system, for Region V (represented in table 1000 as Region V.A), shown in FIGS. 11A-B, gas sources include POC flow and HPO2 gas flow, Q′poc is greater than or equal to Qpoc, max, Qhp=Qout−Qpoc, max, Qpoc, max is greater than Qc, Qpoc=Qpoc, max and Qair=0. However, as shown in table 1050, for a Type B system, for Region V (represented in table 1000 as Region V.B), shown in FIGS. 11C-D, operation differs from that of a Type A system in that Qhp=Qout−Qpoc, and Qpoc is given by Equation 7. In this region of a small range of high FIO2 settings, in a Type A system, as illustrated in FIGS. 11A-B, maximum POC flow is used, along with HPO2 flow for the remainder of the Qout, but the system undershoots, to a degree, the FIO2 settings in this limited region. However, in a Type B system, even in this region, the system can achieve the FIO2 setting without undershooting it, but may do so at the cost using more flow from the limited HPO2 source and potentially requiring greater system complexity.

In FIG. 11A (for an example Type A system), border 1108 represents the border between Regions III and V. This border 1108 represents the threshold beyond which POC flow only is replaced by POC flow and HPO2 flow, for higher FIO2 settings.

FIG. 12A-E-14A-D are plots illustrating examples of contributions from sources of oxygen flow for example Type A, Type B and Type A1 hybrid ventilation systems with a POC with a minimum flow rate of 0.5 L/min and 0.01 L/min, for minute volumes of 2.0 L/min, 8.0 L/min and 16.0 L/min.

FIGS. 12A-E illustrate examples for a minute volume of 2.0 L/min. More particularly, FIG. 12A-B relate to a Type A system, FIGS. 12C-D relate to a Type B system, and FIG. 12E relates to a Type A1 system.

In FIG. 12A (which corresponds with FIGS. 10A and 11A), for a range of FIO2 settings from 21% through 100%, the plot shows the POC oxygen contribution 2004 (at an output gas flow rate of 93% oxygen) and the ambient air contribution 2006 (at 21% oxygen). No HPO2 source contribution is shown, since, for this system and at this minute volume, the range of FIO2 settings only reaches Regions I, II and III, where no flow from the HPO2 source is used. This generally follows since, at low minute volumes, less oxygen supplementation is required, so less or no flow from the HPO2 source may be required.

Broken line 2002 represents the total oxygen (from oxygen supplementation, such as from a POC or HPO2 source, as well as the non-supplementation source of ambient air) that would be used in order to achieve the FIO2 settings without overshooting or undershooting them.

As can be seen, throughout all of in Region I, the total oxygen delivered is steady, with steady contributions from the POC (at minimum flow) and ambient air, which causes overshooting, to a degree, of the FIO2 settings in this Region. To reiterate from previous description, for a Type A system, for this Region, the POC is used at its minimum flow rate, with the remainder of the flow being from ambient air, which conserves HPO2 flow but overshoots, to a degree, the FIO2 setting in this Region.

In Region II, the system achieves the FIO2 setting without overshooting or undershooting it. In this Region, as the FIO2 setting increases, the contribution from the POC increases and the contribution from ambient air decreases (until the FIO2 setting reaches Region III, at which point all delivered flow is provided by the POC). This generally follows since flow from the POC has a greater oxygen concentration than that of ambient air.

In Region III, which includes high FIO2 settings (above the oxygen concentration of flow from the POC, which is, e.g., 93%), the total oxygen delivered is again steady, with all delivered flow being from the POC. As a result, the system undershoots, to a degree, the FIO2 settings in this region, which generally follows since POC flow has an oxygen concentration of, e.g., 93%, rather than, for example, HPO2 source flow with an oxygen concentration of, e.g., 100%.

FIG. 12B (which corresponds with FIGS. 10A and 11B) relates to a Type A system that is in some ways similar to that of the Type A system of FIG. 12A; however, in the system of FIG. 12B, the POC has a minimum flow rate of 0.01 L/min rather than 0.5 L/min. This is approximated, in FIG. 12B, to the POC having no minimum flow rate (or, as described previously, the POC may have a minimum flow rate, but flow from the POC may be diverted to effectively provide delivered POC flows lower than its minimum flow rate). Accordingly, the system of FIG. 12B operates differently than the system of FIG. 12A. Specifically, the system of FIG. 12B has no Region I, but rather, the region that is Region I in the system of FIG. 12A is part of Region II in the system of FIG. 12B. That is because, for the system of FIG. 12B, as FIO2 increases, POC flow gradually increases, and ambient air flow gradually decreases, in order to reach the FIO2 settings in this range without overshooting them, which is possible since the POC is approximated to have no minimum flow rate. Therefore, operation of the system of FIG. 12B in this range, which includes low FIO2 settings, is in accordance with Region II operation.

FIG. 12C (which corresponds with FIGS. 10B and 11C) illustrates a system that is in many ways similar to that of the system of FIG. 12A; however, FIG. 21 illustrates an example of a Type B system instead of a Type A system. Accordingly, as described previously herein, operation differs, relative to the system of FIG. 12A, in Region III, which includes high FIO2 settings. Specifically, in the system of FIG. 12C, in Region III, the system achieves the FIO2 settings without undershooting them, by using a combination of POC flow and HPO2 flow. It follows generally that HPO2 flow increases, relative to POC flow, as FIO2 increases, since flow from the HPO2 source, with an oxygen concentration of, e.g., 100%, has a higher oxygen concentration than flow from the POC, with an oxygen concentration of, e.g., 93%.

The system of FIG. 12D (which corresponds with FIGS. 10B and 11D) operates in some ways similarly to the system of FIG. 12B in that there is no Region I (since there is approximated to be no POC minimum flow). However, the system of FIG. 12D also operates in a way similarly to the system of FIG. 12C with regard to operation in Region III (since it is also a Type B of system).

FIG. 12E (which corresponds with FIGS. 10A and 11B, except for Region I) relates to a Type A1 system (as described above), which is similar in a number of ways to the Type A system of FIG. 12A, including that the POC has a minimum flow rate of 0.5 L/min. However, The Type A1 system of FIG. 12E operates differently in Region I. Specifically, in Region I, a combination of HPO2 flow and ambient air is used (with no POC flow), which allows achieving the FIO2 settings in this region without overshooting them. In this region, as FIO2 increases, more flow from the HPO2 source is used and less ambient air flow is used, which generally follows since higher FIO2 settings require more oxygen supplementation, as achieved including use of HPO2 flow.

FIGS. 13A-D are analogous to FIGS. 12A-D, but illustrate examples for a minute volume of 8.0 L/min rather than 2.0. In particular, FIG. 13A is analogous to FIG. 12A. For this Type A system, for a minute volume of 8.0, there is no Region III but there is a Region V. For this Type A system, in Region V, POC flow is kept steady at its maximum flow rate (of 3.0 L/min), and the remainder of the flow comes from a steady contribution from the HPO2 source. As described previously, this conserves HPO2 flow but causes undershooting, to a degree, of the FIO2 settings in this region.

FIG. 13B is analogous to FIG. 12B, but with a minute volume of 8.0 L/min rather than 2.0 L/min. For this Type A system, for a minute volume of 8.0 L/min, there is no Region III but there is a Region IV and a Region V. In Region IV, delivered flow includes POC flow, HPO2 flow, and ambient air flow. In Region IV, as FIO2 increases, the supplemental oxygen requirement also increases. In this Region, POC flow is kept at its maximum, and HPO2 flow and ambient air flow are also used. However, as FIO2 increases and the supplemental oxygen requirement increases, HPO2 flow is increased while ambient air flow is decreases, which follows since HPO2 flow has a higher oxygen concentration than that of ambient air.

In FIG. 13C, for this Type B of system, in Region V, as FIO2 increases, HPO2 flow gradually replaces POC flow, so that HPO2 flow increases and POC flow decreases in this Region of high FIO2 settings. This allows achieving the FIO2 settings without undershooting them, including use of flow from the HPO2 source (with an oxygen concentration of, e.g., 100%) as necessary to do so, but does not conserve HPO2 flow as much as in the first type of system.

FIG. 13D is analogous to FIG. 12D, but with a minute volume of 8.0 L/min rather than 2.0 L/min. For this Type B system, for a minute volume of 8.0 L/min, there is no Region III but there is a Region IV and a Region V. In Regions II and IV, the system of FIG. 13D operates similarly to the system of FIG. 13B. In Region V, the system of FIG. 13D behaves similarly to the system of FIG. 13C.

FIGS. 14A-D are analogous to FIGS. 13A-D and FIGS. 12A-D, but illustrate examples for a minute volume of 16.0 L/min rather than 8.0 L/min or 2.0 L/min. The systems of each of FIGS. 14A-D operate similarly to the systems of each of FIGS. 13A-D, respectively. However, since the minute volume is higher, the supplemental oxygen requirement is also higher across the range of FIO2 settings, which generally results in shifting of the ranges of FIO2 settings toward lower FIO2 ranges for the various regions, as well as the use of HPO2 flow starting at a lower FIO2 settings.

FIG. 15 is a plot illustrating total oxygen flow across a range of FIO2 settings and minute volumes (where the total flow to the patient, Qout, is equal to the minute volume), for an example hybrid ventilation system, and assuming that the delivered FIO2 matches the FIO2 setting without overshooting or undershooting it. The FIO2 setting is associated with the total oxygen flow, as given by Equation 4, above, such that O2out=FIO2*Qout (which is equal to minute volume). For example, for a FIO2 of 50% and a minute volume of 10, the total oxygen flow is 5 L/min (50%*10 L/min). Generally, as the FIO2 setting (shown on the vertical axis) increases, the oxygen flow requirement increases, and total oxygen flow increases accordingly. Also, as the minute volume increases, the oxygen flow requirement increases, and total oxygen flow increases accordingly. Curves are shown corresponding to various total oxygen flows, in increments of 1 L/min, from 1 L/min to 19 L/min, such as curve 1500, which corresponds to a total oxygen flow of 1 L/min, and curve 1502, which corresponds to a total oxygen flow of 10 L/min and curve. Generally, the higher total oxygen flows are found at higher FIO2 settings and higher minute volumes, where required oxygen supplementation is higher, while the lower total oxygen flows are found at lower FIO2 settings and lower minute volumes, where required oxygen supplementation is lower.

FIGS. 16A-D are plots illustrating example POC flow rates across a range of FIO2 settings and minute volumes, corresponding with FIGS. 11A-D, respectively. In FIG. 16A, for a Type A system with a minimum POC flow of 0.5 L/min, as can be seen, in Region I, the POC flow is at its minimum (0.5 L/min), in Regions IV and V, the POC flow is at its maximum, and in Regions II and III, the POC flow is between its minimum and maximum.

In FIG. 16B, which relates to a Type A system with a minimum POC flow of 0.01 L/min (approximated to zero L/min), and there is no Region I (as described above). As can be seen, the POC flow is at its maximum in regions IV and V, and between its minimum and maximum in Regions II and III.

For FIG. 16C, which relates to a Type B system with a minimum POC flow of 0.5 L/min, the system operates similarly to the system of FIG. 16A in Regions I, II and IV, including with regard to POC flow; However, in regions III and V, less POC flow (and, while not shown in FIG. 16C, more HPO2 flow) is used.

For FIG. 16D, which relates to a Type B system with a minimum POC flow of 0.01 L/min (approximated to zero L/min), the system operates similarly to the system of FIG. 16B in Regions II and IV, but operates similarly to the system of FIG. 16C in Regions III and V, including with regard to POC flow.

FIGS. 17A-B are plots illustrating example HPO2 source oxygen contributions, for Type A and Type B hybrid ventilation systems, with FIG. 17A corresponding to FIGS. 11A-B and FIG. 17B corresponding to FIGS. 11C-D. As can be seen in both of FIGS. 17A-B, for FIO2 settings less than 93% (which matches the oxygen concentration of flow from an example POC), as minute volume increases, flow (and oxygen contribution) from the HPO2 source begins at a lower FIO2 setting (since higher minute volumes require more oxygen supplementation), and, where HPO2 source flow is used, it increases with increasing FIO2 and increasing minute volume (since both require increasing oxygen supplementation).

In FIG. 17A, which relates to a Type A system, for FIO2 settings of more than 93% and low minutes volumes, corresponding to Region III of FIGS. 11A-B, no HPO2 source flow is used (causing undershooting, to a degree, of the FIO2 settings), whereas, in FIG. 17B, which relates to a Type B system, HPO2 source flow is used (to allow achieving the FIO2 settings without undershooting them). Additionally, in FIG. 17A, for FIO2 settings and minute volumes corresponding to Region V of FIGS. 11A-B, less HPO2 source flow is used than in FIG. 17B, for FIO2 settings and minute volumes corresponding to Region V of FIGS. 11C-D, which causes the Type A system of FIG. 17A to undershoot, to a degree, the high FIO2 settings in this region.

FIG. 17C is a plot illustrating example HPO2 source oxygen contributions, for a Type A1 hybrid ventilation system. The plot of FIG. 17C, for a Type B system, differs from that of FIG. 17A (which relates to a Type A system) in that, for low FIO2 settings and minute volumes corresponding to Region I (as shown in FIGS. 11A-B), HPO2 flow is used (but, while not shown in FIG. 17C, no POC flow), which allows achieving the FIO2 settings without overshooting them, whereas, for the Type A system of FIG. 17A, no HP flow is used in Region I (but POC flow is used at its minimum flow, which overshoots, to a degree, the FIO2 settings in this region).

FIG. 18 is a plot illustrating example ambient air oxygen contributions, for a Type A and Type B hybrid ventilation system, corresponding with FIGS. 11A-B. As can be seen in FIG. 18, in Regions III and V (as shown in FIGS. 11A-B and corresponding with table 1000 of FIG. 10A), no ambient air flow is used. However, in all other regions, ambient air flow is used. While not shown in FIG. 18, for a Type B system, as well, in Regions III and V, no ambient air flow is used, but in all other regions, ambient air flow is used.

FIGS. 19A-D are plots illustrating example set versus delivered FIO2, across a range of FIO2 settings and minute volumes, for Type A and Type B hybrid ventilation systems, corresponding with FIGS. 11A-D, respectively.

FIG. 19A corresponds to a Type A system with a minimum POC flow of 0.5 L/min. In Regions III and IV (as shown in FIGS. 11A-B), the system undershoots, to a degree, the FIO2 settings, so that the actual delivered FIO2 is less than the FIO2 setting (since the POC is used at its maximum flow rate of 0.5 L/min, but no HPO2 source flow is used). Also, in Region I, the system overshoots, to a degree, the FIO2 settings, so that the actual delivered FIO2 is greater than the FIO2 setting (with the POC is used at its minimum flow rate of 0.5 L/min).

FIG. 19B corresponds to a Type A system with a minimum POC flow of 0.5 L/min. Like the system of FIG. 19A, the system of FIG. 19B undershoots, to a degree, the FIO2 settings in Regions III and V. However, since, in the system of FIG. 19B, the POC has a minimum flow rate of 0.01 L/min (approximated as zero L/min), the system does not overshoot the FIO2 settings in Region I.

The system of FIG. 19C, which corresponds to a Type B system with a minimum POC flow of 0.5 L/min, like the system of FIG. 19A, overshoots, to a degree, the FIO2 settings in Region I, but, in Regions III and V, unlike the system of FIG. 19A, the system of FIG. 19C achieves the FIO2 settings without undershooting them.

The system of FIG. 19D, which corresponds to a Type B system with a minimum POC flow of 0.01 L/min (approximated to zero L/min), does not overshoot or overshoot the FIO2 settings in any Region. Like the system of FIG. 19B, it does not overshoot the FIO2 settings in Region I (since the POC minimum flow is approximated to zero L/min), and, like the system of FIG. 19C (also a Type B system), it does not undershoot the FIO2 settings in Regions III and V.

FIGS. 20A-B are plots illustrating additional system runtime provided by use of a Type A hybrid ventilation system versus a HPO2 source-only ventilation system (or a hybrid ventilation system with the POC not operational, not available or not used), for a 660 L HPO2 source, across a range of FIO2 settings and minute volumes, with both of FIGS. 20A-B corresponding with both of FIGS. 11A-B. Note that the colormap in FIG. 20A and the y axis in FIG. 20B use a logarithmic scale. In some embodiments of hybrid ventilation systems as described herein, the significant additional runtime that may be provided by including use, or preferential use, of the POC, in addition to use of an HPO2 source, highlights an advantage of such systems, as compared with HPO2 source-only systems. This advantage may be of particular significance, for example, in non-hospital settings with limited HPO2 availability and/or multiple patients requiring mechanical ventilation and oxygen supplementation.

In FIG. 20A, it can be seen that, in Regions I, II and III (as shown in FIGS. 11A-B), where POC flow alone is sufficient for oxygen supplementation for this Type A system (at a maximum oxygen concentration of, e.g., 3.0 L/min and an oxygen concentration of, e.g., 93%), since the POC can provide up to its maximum flow rate for an unlimited period of time, additional runtime in these regions is likewise unlimited. In Regions IV and V, it can be seen that including flow from the POC, in addition to flow from the HPO2 source, can provide substantial additional system runtime, which additional runtime increases dramatically as FIO2 settings and minute volumes decrease in these regions.

FIG. 20B shows curves representing additional runtime provided at particular minute volumes from 5.0 L/min to 15.0 L/min, for various FIO2 settings, for a Type A system. As can be seen in FIG. 20B, as FIO2 decreases, additional provided runtime increases, which results in each of the curves rising with respect to the vertical axis as FIO2 decreases, and rising most dramatically as operation approaches the thresholds of the regions in which regions the additional runtime is unlimited, as described above. Since lower minute volumes also result in greater additional runtime, curves for lower minute volumes are higher, relative to the vertical axis, for particular FIO2 settings, indicating greater provided additional runtime.

FIGS. 20C-D are plots illustrating additional system runtime provided by use of a Type B hybrid ventilation system versus a HPO2 source-only ventilation system, across a range of FIO2 settings and minute volumes, with both of FIGS. 21C-D corresponding with both of FIGS. 11C-D. Note that the colormap in FIG. 20C and the y axis in FIG. 20D use a logarithmic scale.

The system of FIG. 20C (a Type B system) is similar to the plot of FIG. 20A (a Type A system) except for Regions III and V (as shown in FIGS. 11C-D). In Region III, the system of FIG. 20C provides limited additional runtime (since HPO2 flow is used), whereas the system of FIG. 20A provides unlimited additional runtime (since no HPO2 flow is used). Also, in Region V, the system of FIG. 20C provides less additional runtime than the system of FIG. 20 A, since more HPO2 source flow is used.

FIG. 20D is analogous to FIG. 20B, except that it relates to the Type B system of FIG. 20C, rather than the Type A system of FIG. 20A. In accordance with the description of FIG. 20C, above, it can be seen in FIG. 20D that, for lower FIO2 settings and minute volumes (corresponding with Regions III and V), less additional runtime is provided, with increasingly less additional runtime being provided as the FIO2 setting approaches 100%.

FIG. 20E is similar to FIG. 20A, except that it relates to a Type A1 system rather than a Type A system. Note that the colormap in FIG. 20E uses a logarithmic scale. Accordingly, for the system of FIG. 20E, in Region I (as shown in FIGS. 11A-B), a limited amount of additional runtime is provided (since HPO2 flow is used), as compared with the unlimited amount of runtime provided in the system of FIG. 20A in this region (since no HPO2 flow is used).

FIGS. 21A-C are plots illustrating example flow rates and ranges by region for a Type A hybrid ventilation system, with a POC minimum flow rate of 0.5 L/min and maximum flow rate of 3.0 L/min, corresponding with FIG. 11A, for minutes volumes of 3.5, 8.0 and 16 L/min, respectively.

As can be seen in FIGS. 21A-C, as minute volume increases, the FIO2 range of Region I extends to increasingly narrow FIO2 ranges with increasingly lower upper FIO2 boundaries. As minute volume increases, more supplemental oxygen is needed to achieve a particular FIO2 setting. As such, as minute volume increases, the minimum POC flow (of, e.g., 0.5 L/min) will achieve an increasingly lower FIO2 setting. As a result, as minute volume increases, the minimum POC flow will overshoot the FIO2 setting at increasingly lower FIO2 settings.

As can be seen in FIGS. 21A-C, for Region II, the FIO2 settings are achieved using a POC flow between its minimum and maximum, and no HPO2 source flow is used (so, Qpoc is between Qpoc, min and Qpoc, max, and Qhp=0). As minute volume increases, the FIO2 range of Region II extends to narrower ranges with increasingly lower FIO2 boundaries. As minute volume increases, more supplemental oxygen is needed to achieve a particular FIO2 setting. As such, as minute volume increases, the range of FIO2 settings achieved with POC flows between its minimum and maximum will generally cover increasingly lower FIO2 settings. In FIG. 21A, example boundary point 1314 represents the boundary between regions II and IV, at a minute volume of 3.5 L/min, at which point the FIO2 setting (approximately 85%) can be achieved with the POC at its maximum flow (Qpoc, max=3.0 L/min).

In FIGS. 21A-C, none of the example minute volumes extend into Region III. However, Region III is nonetheless shown and described, as it would apply to minute volumes below 3.0 L/min at high FIO2 settings. For this Type A system, In Region III, the POC flow is set equal to Qout but no HPO2 source flow is used (so, Qpoc=Qout, Qhp=0), which results in undershooting, to a degree, of the FIO2 settings in this region.

As can be seen in FIGS. 21A-C, for Region IV, the FIO2 setting is achieved using a POC flow at its maximum and using HPO2 flow as needed (so, Qpoc=Qpoc, max, Qhp>0). As minute volume increases, the FIO2 range of Region IV changes such that its upper boundary increases relatively slowly while its lower boundary decreases more rapidly, so that the overall range widens. Since the POC flow has an example oxygen concentration of 93%, and a maximum flow rate of 3 L/min, at a minute volume of 3 L/min, maximum flow from the POC can achieve a FIO2 of 93%. However, the POC maximum flow is equal to the total flow (Qout), so no HPO2 flow can be added to achieve any higher FIO2 setting. However, as minute volume increases beyond 3 L/min, HPO2 flow can be added to the POC maximum flow to achieve increasingly higher FIO2 settings. Therefore, the upper boundary of Region IV increases with increasing minute volumes beyond 3.0 L/min. The lower boundary of Region IV, however, decreases with increasing minute volume beyond 3.0 L/min. This follows since, as more oxygen supplementation is required to achieve a particular FIO2 setting as minute volume increases, the maximum POC flow, without HPO2 flow added, can achieve only lower FIO2 settings.

In Region V, which includes only high FIO2 settings at minute volumes greater than 3.0 L/min, the POC is used at its maximum flow and some HPO2 source flow is used (so, Qpoc=Qpoc, max, Qhp>0), which undershoots, to a degree, the FIO2 settings in this region.

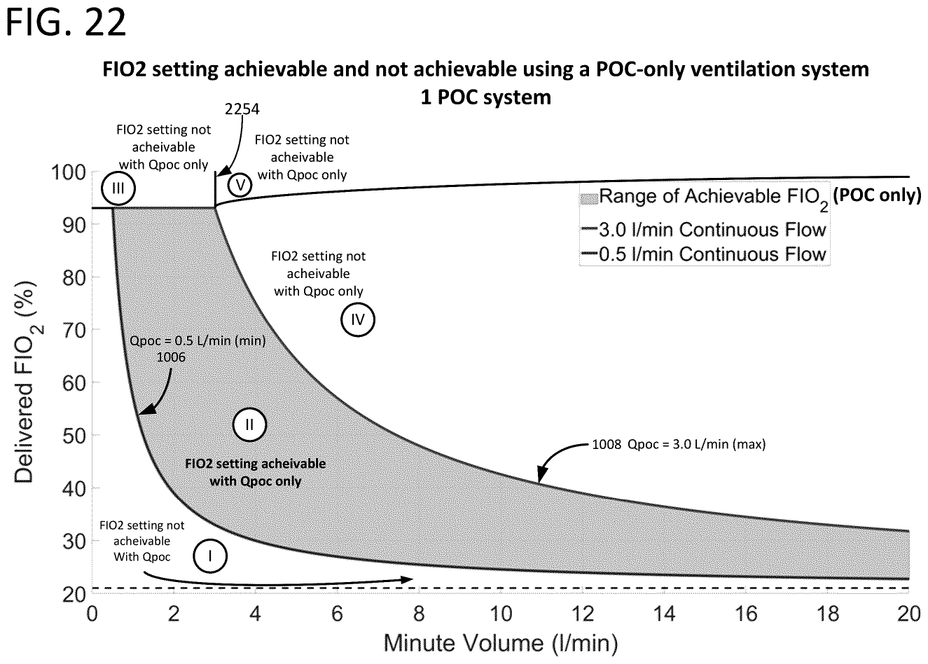

FIG. 22 is a plot illustrating FIO2 settings achievable and not achievable using a system with a POC but no HPO2 source or other supplemental oxygen source (or a hybrid ventilation system with the HPO2 source exhausted, unavailable, or not being used). The regions shown in FIG. 22 correspond with the regions as shown in FIGS. 11A-B. In some embodiments of a hybrid ventilation system including use of a POC and HPO2 source, however, all FIO2 settings are achievable. This highlights an advantage of embodiments of hybrid ventilation systems as described herein, which allow achieving a wide range of FIO2 settings for a wide range of minute volumes, and yet use POC flow to conserve HPO2 supply.

As shown in FIG. 22, with use of a POC only as an oxygen supplementation source, only the FIO2 settings and minute volume combinations of Region II can be achieved, using POC flow between its minimum flow rate (e.g., 0.5 L/min) and maximum flow rate (e.g., 3.0 L/min). However, some embodiments of hybrid ventilation systems described allow reaching all FIO2 and minute volume combinations (or, in some embodiments, overshoots or undershoots, to a degree, FIO2 settings in some regions, which may allow greater HPO2 source conservation and system simplicity), but may also use, or preferentially use, POC flow to conserve the HPO2 source (thereby potentially allowing use of a smaller, lighter HPO2 source, and/or extending overall system runtime). As shown in FIG. 22, curve 1006 represents the boundary between Regions I and II, where the FIO2 setting can be achieved with the POC at its minimum flow of, e.g., 0.5 L/min, and curve 1008 represents the boundary between regions II and IV, where the FIO2 setting can be reached with the POC at its maximum flow (e.g., 3.0 L/min).

As also shown in FIG. 22, none of the FIO2 settings and minute volume combinations of the other Regions, including Regions I, III, IV and V can be achieved with the system using a POC as the only oxygen supplementation source, whereas, in embodiments of hybrid ventilation systems as described herein, the FIO2 settings can be achieved even in these regions.

FIG. 23 illustrates an embodiment including settings achievable using a hybrid ventilation system with two POCs but no HPO2 source, while all FIO2 settings are achievable using some example hybrid ventilation systems. The regions I-VI as shown in FIG. 23 are analogous with regions I-VI as shown in FIG. 11C (for a type B system, as described previously herein, including with reference to FIG. 9). However, FIG. 23 relates to a system including 2 POCs, whereas the system in FIG. 22 relates to a system including a single POC. As such, in FIG. 23, region II includes potential contributions from both of the POCs. Specifically, as depicted, region II is divided into region portion IIa and region portion IIb. Region portion IIa includes the range of FIO2 settings (relative to minute volume) achievable using one of the two POCs, while region IIb includes the range of FIO2 settings achievable using both POCs. Since each POC contributes a maximum of 3.0 L/min, both POCs together can contribute up to 6.0 L/min. In some embodiments, rather than 2 (or more) POCs, fewer POCs, or one POC, may be used, such one or more larger or higher volume POCs, that have higher maximum outputs (e.g., 4.0 L/min, 5.0 L/min, 6.0 L/min, 9.0 L/min, 12.0 L/min, or more).

The non-vertical curve portion 2354, in FIG. 23, between regions III and V, is consistent with a Type B hybrid ventilation system, as shown in FIG. 11C and described herein. In FIG. 22, the vertical line portion 2254, between regions III and V, is consistent with a Type A hybrid ventilation system, as shown in FIG. 11A and described herein. As described in detail, e.g., with reference to FIG. 9, Type A and Type B systems differ with respect to operation in regions II and V. It is to be noted, however, that, while not shown in FIGS. 22 and 23, a single POC system, such as illustrated in FIG. 22, could alternatively be implemented as a Type B system, and a multiple POC system, such as that shown in FIG. 23, could alternatively be implemented as a Type A system.

As can be seen, use of two (or more) POCs, by providing potentially double (or more) the output of a single POC, may allow a range FIO2 settings to be achieved at higher minute volumes than with use of a single POC. Similarly, use of two (or more) POCs, for each of a range of FIO2 settings, allows higher minute volumes to be provided. As such, use of multiple POCs (and/or higher output POCs) may provide sufficient oxygenation (e.g., without use of an HPO2 source, or with less use thereof) to accommodate a greater range of patient(s) conditions and needs, such as may include, e.g., potentially uncommon situations or time periods in which a greater than usual FIO2 setting or higher minute volume may be required, for example.

In the embodiment depicted, the first POC could be used to achieve a maximum FIO2 setting as shown by curve 2352, outputting the maximum 3.0 L/min. To achieve the FIO2 settings shown in region IIb, at least a portion of the maximum possible output from the second POC would need to be used as well, up to the maximum FIO2 setting as shown by curve 2355, with the second POC outputting the maximum 3.0 L/min. In various embodiments, various combinations of outputs from each of the two POCs could be used to achieve various different FIO2 settings in region II, such that a combined output of between 0.5 L/min (minimum output from one POC only) to a maximum output of 6.0 L/min (maximum output from both POCs). For example, to achieve an output of 1.0 L/min, one POC could output 1.0 L/min, one POC could contribute 0.5 L/min and the other could contribute 0.5 L/min, etc.

Generally, the output from each of the two POCs may be chosen to meet the needed total output, and the contribution from each POC may be based on any of a variety of factors, or one or more algorithms may be used—e.g., if one POC is not operational, then the other POC may be used, or one POC may be used more than the other, etc. Additionally, in various embodiments, more than two POCs may be used (e.g., three, four, five or more). In general, N POCs may be used, where N is the number of POCs used, and where the total POC oxygen contribution would be the total output from all N POCs, and the maximum available POC output at a given time would be the total output at that time for all N POCs (assuming they are all available). For example, if each POC has a total output of 3.0 L/min, then the total maximum output at a given time may be given by N×3.0 L/min. For example, if three POCs are used, each with an output range of 0.5 L/min-3.0 L/min, then the total output could range from 0.5 L/min (minimum output from one of the POCS) to 9.0 L/min (maximum output from all of the POCS). If three POCs are used, then, relative to the regions as shown in FIG. 23, there would be a region IIc adjacent to region IIb, showing achievable FIO2 settings (relative to minute volume) from 6.0 L/min-9.0 L/min (maximum output from all three POCs). Furthermore, in some embodiments, in a multiple POC system, all of the POCs may have outputs that are controlled by or within the system, or one or more POCs may have outputs that are controlled or adjusted by or within the system while one or more other of the POCs may have outputs that are manually controlled or adjusted.

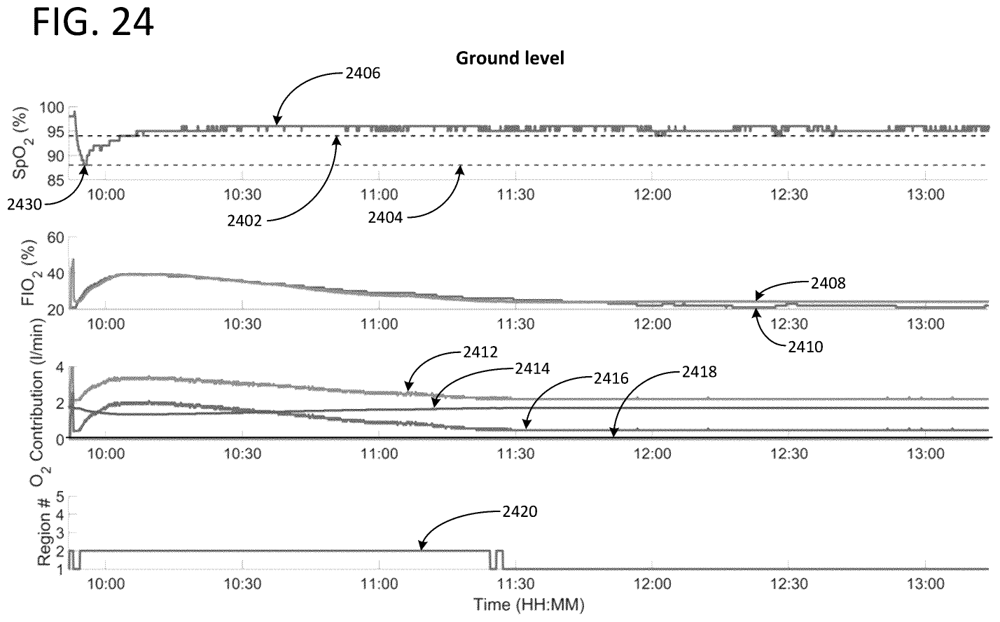

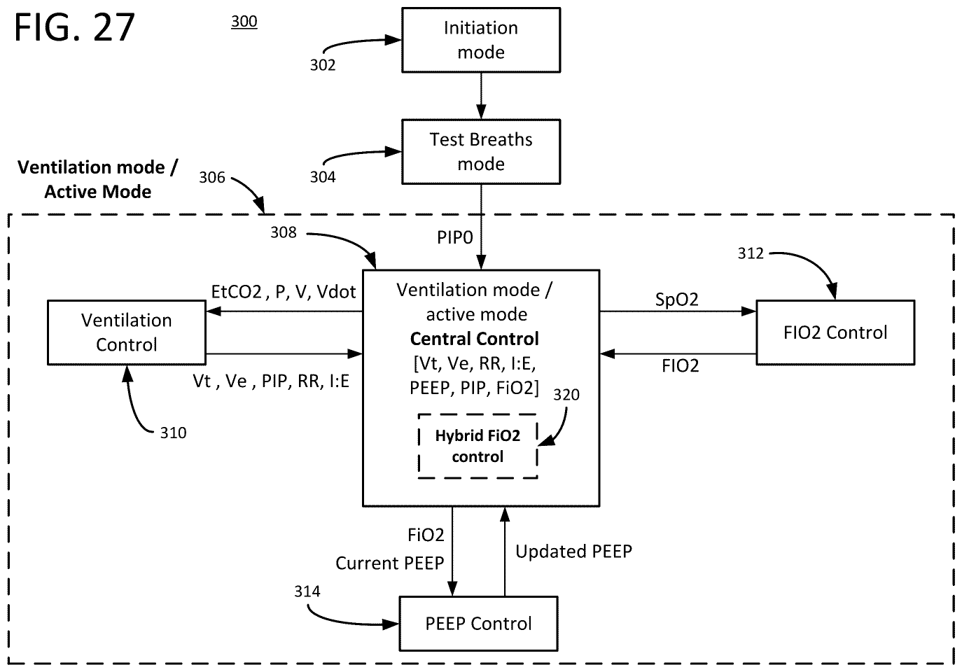

FIGS. 24-26 include plots relating to performed animal studies illustrating use of a hybrid ventilation system in accordance with an exemplary embodiment as described herein to maintain normoxia after lung injury at ground level, a simulated altitude of 8,000 feet, and a simulated altitude of 16,000 feet, respectively. In particular, the hybrid system used included one POC with an output range of 0.5 L/min-3.0 L/min, and an HPO2 source was included and available in the system. In each case, as illustrated in FIGS. 24-26, a porcine model was used. Following lung injury and bringing the animal to the appropriate simulated altitude, if any, a closed loop control algorithm (an example of which is described with reference to FIG. 27 herein) was used in adjusting the FIO2 setting based on a target SpO2 of 94%. It is noted that, in each of FIGS. 24-26, the relevant period of study is the period following the desaturation condition, which may be associated with a lung injury or other condition, occurring very early in each period.

Overall, as further described as follows, FIGS. 24-26 demonstrate successful and proper operation of the exemplary hybrid ventilation system (Type B), in multiple regions of operation, and including use of an HPO2 source and POC, for animals that have sustained, and then begin to recover over time from, a lung injury, at ground level as well as at simulated altitudes of 8,000 and 16,000 feet. This demonstrates the capability of embodiments of hybrid ventilation systems, as described herein, to successfully provide, and to provide technical solutions to problems relating to, oxygenation support to patients, including with lung injuries or, for example, other conditions affecting effective or optimal oxygenation from breathing, including at altitudes, and with changes in altitudes, that may be encountered during aeromedical transport and return to sea level. Furthermore, treatment can be optimized in situations in which HPO2 source output is limited (e.g., portable HPO2 source supply may be low), and/or a smaller supply of HPO2 may be required to be carried, reducing the associated burden and risks of carriage and transport. In some embodiments, as described, closed loop control, such as of FIO2, can be successfully used in such systems, for continuous monitoring and adjustment, optimizing aspects of treatment while reducing manual care provider burden in challenging care scenarios. These solutions and advantages may prove especially valuable in challenging care scenarios, including (but not limited to) pre-hospital, field, transport, high patient volume, disaster and military scenarios, for example.

In the plots shown in FIG. 24, the animal is at ground level. Plots are included that relate to, over the time period of the study (with the times being shown), the animal's SpO2(%), the FIO2 setting (%), oxygen contributions from various sources, and the region of operation of the system (corresponding with the regions shown in FIG. 11C).

With regard to SpO2, curve 2406 shows measured SpO2, dotted line 2402 shows target SpO2 (94%), and dotted line 2404 shows the desaturation threshold (defined in the study as an SpO2 of 88%). As can be seen, early in the time period shown, SpO2 decreases down to the desaturation threshold of SpO2 of approximately 88% 2340, SpO2 rises until, throughout the remainder of the time period shown, SpO2 is maintained at or above an SpO2 of 94%. This indicates that the hybrid ventilation system is working properly.

With regard to FIO2, curve 2408 shows measured FIO2 and curve 2410 shows FIO2 setting. As can be seen, a good agreement between measured FIO2 and FIO2 setting is maintained throughout the time period shown, indicating that the hybrid ventilation system is working properly. Furthermore, as expected, after SpO2 decreases, the FIO2 setting (and measured FIO2) increases to bring the animal's SpO2 at or above 94%. For the remainder of the time period shown, a gradually lower FIO2 is required to maintain the animal's SpO2 at or above 94%. This may indicate that the animal is slowly recovering or improving over time, and therefore in need of gradually less oxygen support.

With regard to oxygen contributions, curve 2412 shows total oxygen contribution from all sources (air, POC and HPO2 source), curve 2414 shows the air contribution, curve 2416 shows the POC contribution, and line 2418 shows the HPO2 source contribution. In the time period shown, and at ground level, no contribution from the HPO2 source was required, so line 2418 remains at 0 L/min throughout the time period shown. As can be seen, from desaturation 2430, output from the POC increases until the animal's SpO2 reaches at or above an SpO2 of 94%. For the remainder of the time period shown, the output from the POC slowly decreases, and then stays about level, as a lower FIO2 is required to maintain the animal's SpO2 at or above an SpO2 of 94%. After the SpO2 of at or above 94% is achieved, the oxygen contribution from air gradually increases, and then stays about level. The oxygen contribution curves 2412-2418 indicate that the hybrid ventilation system is working properly.

With regard to region of operation, throughout the time period shown, the system operates in either region I (as depicted in FIG. 11C), with Qpoc=Qpoc,min (0.5 L/min), or region II, where Qpoc is between Qpoc,min (0.5 L/min) and Qpoc,max (3.0 L/min). In particular, generally, during most of the first part of the time period, the system operates in region II, but, during approximately the second half of the time period, the system operates in region I with only the minimal flow from the POC (0.5 L/min) being used, with lower FiO2 being required. Overall, in FIG. 24, the curves/line 2402-2420 show that the hybrid ventilation system is working properly, following lung injury and with the animal at sea level.

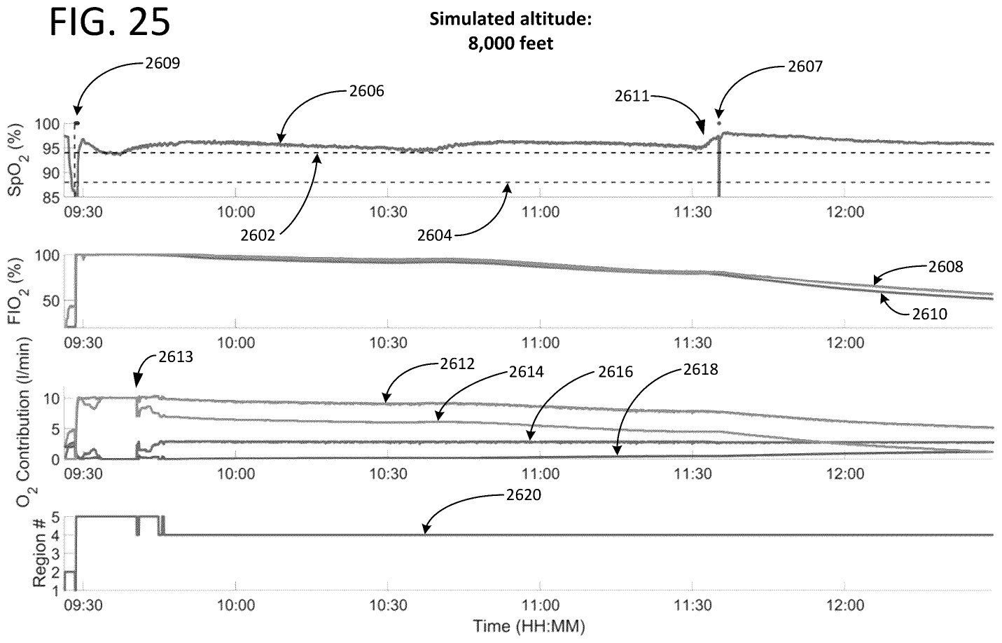

In the plots shown in FIG. 25, the animal is at a simulated altitude of 8,000 feet until time 2611, when the animal is transferred to sea level. Curves/line 2602-2620 represent similar parameters, for the animal at a simulated altitude of 8,000 feet, as those represented by curves/line 2402-2420 of FIG. 24, for the animal at ground level. Similar to FIG. 24, the curves 2602-2620 of FIG. 25 show that the hybrid ventilation system is working properly, following lung injury and with the animal at a simulated altitude of 8,000 feet throughout a large portion of the time period shown.

Similar to FIG. 24, in FIG. 25, as shown by curve 2606, following desaturation 2609, the animal's SpO2 is successfully increased to, and then maintained, at or above 94%. The increase in SpO2 as represented by feature 2611, occurs immediately after the animal is transferred from simulated altitude back to ground level, resulting in temporarily increase in SpO2, and the closed loop control algorithm successfully maintains the animal's SpO2 at or above 94%. The apparent sudden dip in SpO2 at time 2607 is caused by the SpO2 sensor (pulse oximeter) being removed from the animal for a short period of time, and does not actually represent a sudden dip in SpO2.

Similar to FIG. 24, in FIG. 25, the FIO2 setting curve 2610 and the measured FIO2 curve show good agreement throughout the time period shown.

In FIG. 25, unlike in FIG. 24, as indicated by oxygen contribution curves 2612-2618, the HPO2 source is properly required for a portion of the time period shown. In particular, In FIG. 25, it can be seen that, for a portion of time following desaturation 2609, the HPO2 source (curve 2614) is used rather than the POC (curve 2616), and for the remainder of the time period thereafter, the HPO2 source and the POC are both used, with the contribution from the HPO2 source slowly decreasing over time, while the contribution from the POC stays approximately level, as the animal's need for support gradually decreases over time.

As illustrated in FIG. 11C and described herein, for a period of time following desaturation 2609, the system operates first in region V then varies between operating in region V and region IV, and then eventually operates only in region IV for the remainder of the time period shown. It can be seen that, for the time period generally indicated by feature 2613, the HPO2 source supplies all or most of the oxygen supplementation, and the POC supplies none or the remainder. Later in the time period shown, operating in region IV, both the HPO2 source and the POC contribute, with the HPO2 source output gradually decreasing, and the POC contribution remaining at its maximum output of 3.0 L/min. The oxygen contribution curves 2612-2618 indicate proper operation of the system.

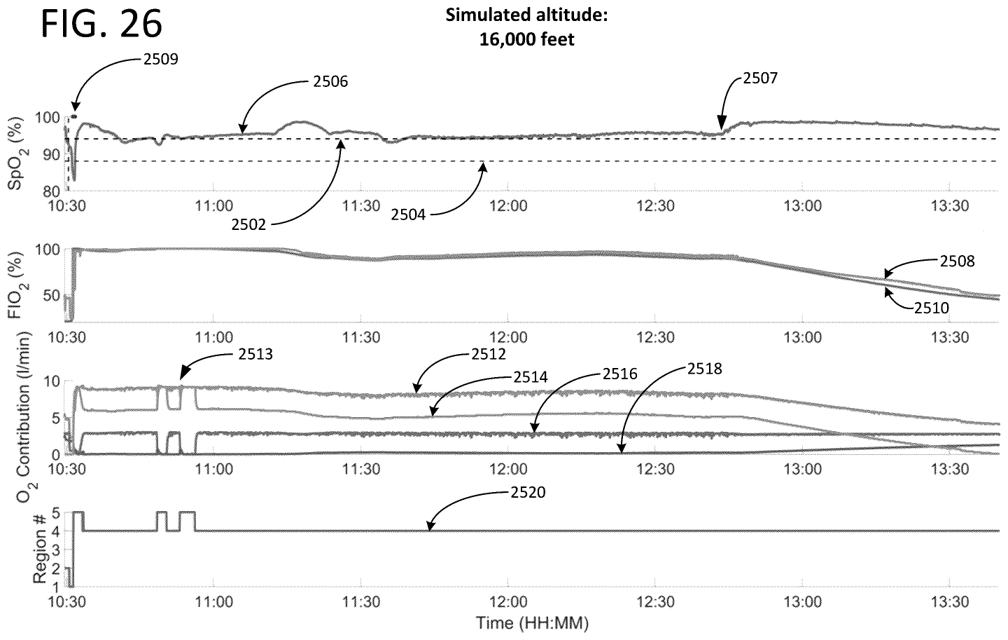

In the plots shown in FIG. 26, the animal is at a simulated altitude of 16,000 feet until time 2507, when the animal is transferred to ground level. Curves/line 2502-2520 represent similar parameters, for the animal at a simulated altitude of 16,000 feet, as those represented by curves/line 2602-2620 of FIG. 25, for the animal at a simulated altitude of 8,000 feet. Similar to FIG. 25, the curves 2502-2520 of FIG. 26 show that the hybrid ventilation system is working properly, following lung injury and with the animal at a simulated altitude of 16,000 feet throughout a large portion of the time period shown. In particular, following desaturation 2509, generally, the SpO2 2506 of the animal is increased to 94%, and then maintained at or above 94%, and the measured FIO2 2508 and FIO2 setting 2510 show good agreement. Generally similar to that of FIG. 25, operation of the system varies between regions 4 and 5, as shown in the general time period indicated by feature 2513, then operation remains in region 4 for the remainder of the time period.

As expected, as simulated altitude increases, greater oxygen supplementation is generally required, resulting in a greater FIO2 being required. In FIGS. 24-26, this is reflected in the measured FIO2 and FIO2 setting curves, where, at a simulated altitude of 8,000 feet, a generally higher FIO2 is required than at sea level, and, at a simulated altitude of 16,000 feet, a generally higher FIO2 is required than at 8,000 feet.

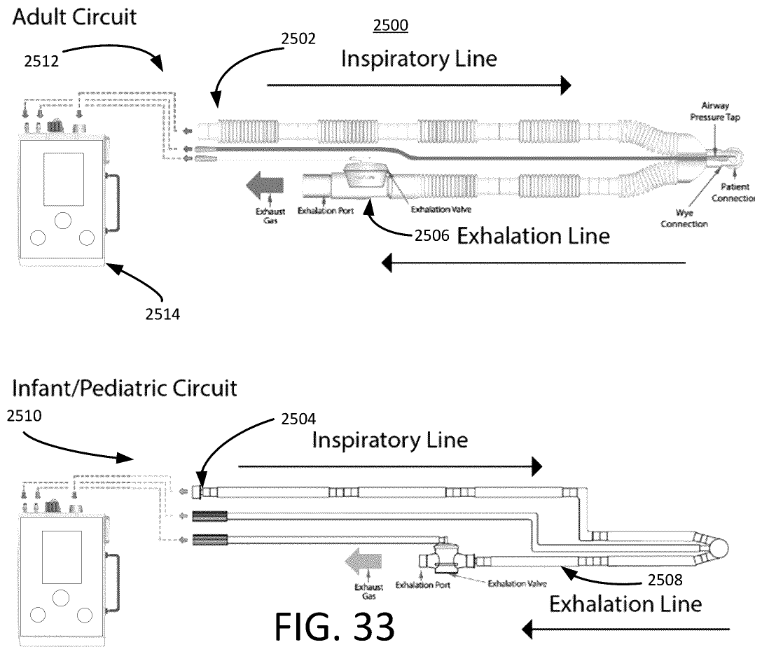

FIG. 27 is a block diagram illustrating example conceptual or software architecture-related aspects 300 of a ventilation system incorporating closed loop control, in accordance with embodiments of the present disclosure, which may be implemented by a controller, such as the controller 2750 of the portable ventilator as shown in FIG. 33, or the controller of one or more devices as depicted in FIG. 1, for example. An initiation mode 302, a test breaths mode 304 and an active mode 306 are shown. During active mode, implemented using an active mode central control 308, the controller may cause delivery of ventilation to the patient, such as, for example, via a gas delivery apparatus and using a facemask, coupled with the patient, or intubation, using ventilation parameters that are continuously updated, which may include FIO2 and PEEP. In some embodiments, however, the particular exemplary modes of FIG. 27 may not be used. As depicted, the active mode central control 308 also includes a hybrid FIO2 control 320, embodiments of which are described previously herein.

In initiation mode 312, the user may be prompted to enter the patient's gender and height, which may be used in determining a bodyweight associated with the patient, or “predicted” bodyweight (PBW). Based on the determined patient's PBW, several ventilation parameter settings may be determined for use in test breaths mode 304.

In various embodiments, in initiation mode 302, various calculations may be utilized to determine test breaths parameter settings. In one example, in porcine use and studies, tidal volume (Vt) (in ml) may be calculated as 10*actual weight in kg, minute volume (in ml/min) as actual weight in kg*140, and respiratory rate (RR) as minute volume/Vt, where RR may be rounded to the next higher integer value. However, various other calculations and values may be used in various embodiments.

In another example, in human use, the following calculations may be utilized. The PBW (in kg) may be calculated as C+D*(height in cm−E), where C may be, e.g., between 35-60, D may be, e.g., between 0.80 and 1.00, and E may be, e.g., between 140-160, and where, in some embodiments, at least C may be slightly lower for a female than for a male. For Vt greater than or equal to, e.g., a value between 150-250 ml, rounding may be performed to, e.g. the nearest 1-10 ml, 3-7 ml, and for Vt less than or equal to, e.g., a value between 15-200 ml, rounding is performed to, e.g., the nearest 0.5-5 ml, 0.5-3 ml. However, various other calculations and values may be used in various embodiments, such as various calculations that are based on, or functions of, height, gender and/or one or more other patient physical or health-related characteristics.

In some embodiments, some or all of the results of the calculations made in initiation mode 302 are displayed to the user. The user may, for example, be prompted to accept the determined parameters and then couple the patient to the ventilator.

In some embodiments, following completion of initiation mode 302, the system 300 proceeds to test breaths mode 304. In test breaths mode 304, the system 300 may deliver one or several ventilation test breaths to the patient, based on which the system 300 may determine one or more patient parameters and/or particular ventilator settings to utilize at the start of active mode 306. For example, in some embodiments, in the test breaths mode 304, a patient respiratory system compliance (Crs), such as an estimated patient Crs, is determined and used in determining a peak inspiratory pressure (PIP(0)) ventilator setting. In some embodiments, Crs may be calculated or estimated using patient respiratory dynamics data and the equation of motion for the respiratory system. Furthermore, in some embodiments, in test breaths mode 304, the following particular parameter settings may be utilized: inspiratory:expiratory ratio (I:E)=1:3, PEEP=5 cm H2O, FIO2=0.5 (or 50%). However, in various embodiments, other initial settings may be used.

Following successful completion of the test breaths mode 304 and determination of the patient Crs, the system 300 may proceed to active mode 306. In some embodiments, the system may proceed to active mode 306 even if patient Crs cannot be determined. For example, in some embodiments, if Crs cannot be determined, a default Crs value may be utilized, such as a Crs value of 50-150 ml/cm H2O, 70-130 ml/cm H2O, 80-120 ml/cm H2O, 90-110 ml/cm H2O, for example. In some embodiments, the default value may be determined to be relatively high, since, in some embodiments, that will result in relatively small changes in a PIP correction value thus providing a relatively conservative approach.

In some embodiments, during active mode 306, the system 300 delivers continuously adjusted ventilation to the patient, which may include closed loop control of one or more patient and/or ventilation related parameters.

In some embodiments, during active mode 306, a number of ventilation parameters are continuously adjusted, including FIO2 and PEEP. Other parameters that are adjusted during ventilation mode 308 in a continuous manner may include PIP, Vt, Ve, RR and I:E. In some embodiments, FIO2 is continuously adjusted based on a target patient oxygenation level, such as an SpO2 of 94%. In some embodiments, FIO2 starts at 21% (or, e.g., 21-25%). However, some embodiments, a user may select an initial FIO2 setting, such as between 21%-100%, for example.

On a continuous basis, central control 308 may provide data to ventilation control 310, including current EtCO2 as well as pressure (P) and volume (V) waveforms, and/or parameters derived at least in part from the P and V waveforms. Ventilation control 310 may use that data, potentially in addition to other data, in determining values for Vt, Ve, PIP, RR and I:E, which it then sends to central control 308. Central control 308 may then implement any appropriate adjustments to Vt, Ve, PIP, RR and I:E settings based at least in part on the sent values.

Furthermore, on a continuous basis, central control 308 may provide data to FIO2 control 312, including current patient oxygen saturation (SpO2). FIO2 control 310 may use that data, potentially in addition to other data, in determining a value for FIO2, which it then sends to central control 308. Central control 308 may then implement any appropriate adjustment to the FIO2 setting based at least in part on the sent values. Adjustment of the FIO2 setting may be accomplished via appropriate actuation of an oxygen source valve, or by adjusting an oxygen concentration from an oxygen supply, or in other ways.

Still further, on a continuous basis, central control 308 may provide data to PEEP control 314, including the current FIO2 and current PEEP. PEEP control 314 may then use that data, potentially in addition to other data, in determining an updated value for PEEP. Central control 308 may then implement any appropriate adjustment to the PEEP setting based at least in part on the sent value. Adjustment of the PEEP setting may be accomplished via appropriate actuation of an exhalation valve, for example.

It is to be understood that, while central control 308, ventilation control 310, FIO2 control 312 and PEEP control are described separately and communication with each other, in some embodiments, some or all of these may be combined or integrated, or may function independently. In such embodiments, communication between combined or integrated components may be less or may be unnecessary.

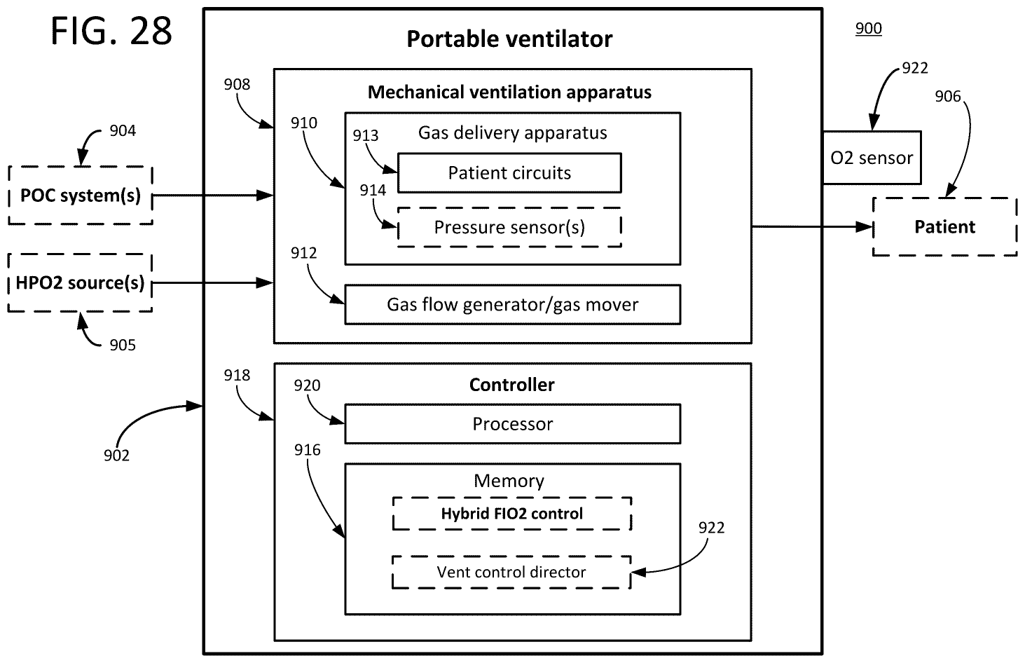

FIG. 28 illustrates an example portable ventilator 902, in accordance with some embodiments, capable of providing ventilation, and potentially other interventions or treatments, to a patient 906. The portable ventilator 902 includes a mechanical ventilation apparatus 908 and at least one controller 918. The controller 918 includes at least one processor 920 and at least one memory 916 for storing data, and may also include software that may be stored in the at least one memory 916, such as may include, or may include aspects of, a ventilation control director 922 and a hybrid FIO2 control 924, embodiments of which are previously described herein. The portable ventilator 902 may include or be connected to supplemental oxygen sources, which may include one or more POC systems 904 and one or more HPO2 sources 905. In some embodiments, the system 900 also includes an O2 sensor 922 (or O2 sensing component including an O2 sensor), as described with reference to FIG. 1, that may be, e.g., coupled with the inspiration limb of the patient circuit, or elsewhere.

In some embodiments, the portable ventilator 902 is capable of sensing signals representative of gas flow that can be used in determining at least one patient respiratory parameter, such as may include use or one or more pressure sensors 914, flow sensors, pneumotachometers or spirometers, that may, in some embodiments, be included as part of, or within or partially within, the mechanical ventilation apparatus 908, or may be separate but communicatively coupled by wired or wireless connection. In various embodiments, one or more spirometers may be included within the portable ventilator 902 and/or may be coupled (physically and/or communicatively by wired or wireless connection) with the portable ventilator 902. Although depicted in a separate box from the patient circuits 913, it is to be understood that the pressure sensors 914 and/or other components such as flow sensors, pneumotachometers or spirometers, may be included within or partially within the patient circuits 913, such as a patient inspiratory circuit and/or a patient expiratory circuit. In some embodiments, the pressure sensor(s) 914 and other associated components may be used in sensing or obtaining respiratory parameter data, which respiratory parameter data may be used in determining or generating a respiratory status (which can include associated data). The respiratory parameter data and respiratory status data may also be used in connection with control of operation of the portable ventilator 902, such as in control of mechanical ventilation providing by the portable ventilator 902, whether such control is internally provided by the portable ventilator 902, remotely provided, or with aspects of both.

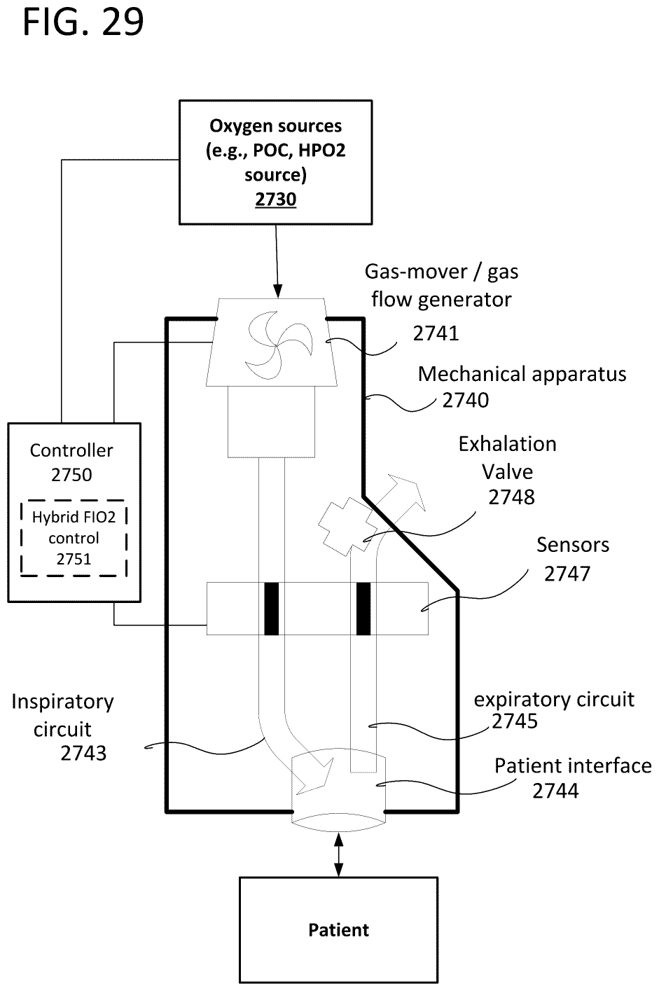

FIG. 29 illustrates aspects of an example of the mechanical ventilation apparatus for a ventilation system such as a portable ventilator, including a controller 2750 and coupled with one or more supplemental oxygen sources 2730 (e.g., a pressurized oxygen source, such as a HPO2 stank, and an oxygen concentrator or POC), used in the providing mechanical ventilation to a patient or patients. As depicted, the controller 2750 includes a hybrid FIO2 control 2751, embodiments of which are previously described herein.

The patient interface 2744 may include an appropriate gas delivery device, such as an intubation tube, mask, nasal cannula, etc. The mechanical ventilation apparatus 2740 further includes an expiratory line 2745 and an exhalation valve 2748 and a heat and moisture exchanger. Both the inspiratory line 2743 and the expiratory line 2745 include sensors 2747. The sensors 2747 may include, for example, but not limited to, the pneumotachometer 275, the airway pressure sensor 274, and a spirometer. Sensors 2747 enable the ventilation system to measure the patient's respiratory efforts as well as the performance of the ventilation system when providing mechanical respiratory assistance to the patient. The sensors 2747 may generate and provide the data including but not limited to flow rate, tidal volume and minute volume, respiratory mechanics (e.g., resistance and compliance) and spirometry, and may include, for example, forced vital capacity (FVC), forced vital capacity at 1 second (FEV1) and peak expiratory flow rate (PEF or PEFR). In addition, a patient monitor/defibrillator or another medical device may provide, for example, capnography and/or oximetry data, such as oxyhemoglobin and carboxyhemoglobin saturation and mainstream or other capnographic data such as end tidal CO2 (EtCO2). This data may allow, for example, for calculation of CO2 elimination rate and volumetric capnography, which may include using flow data from the ventilation system.

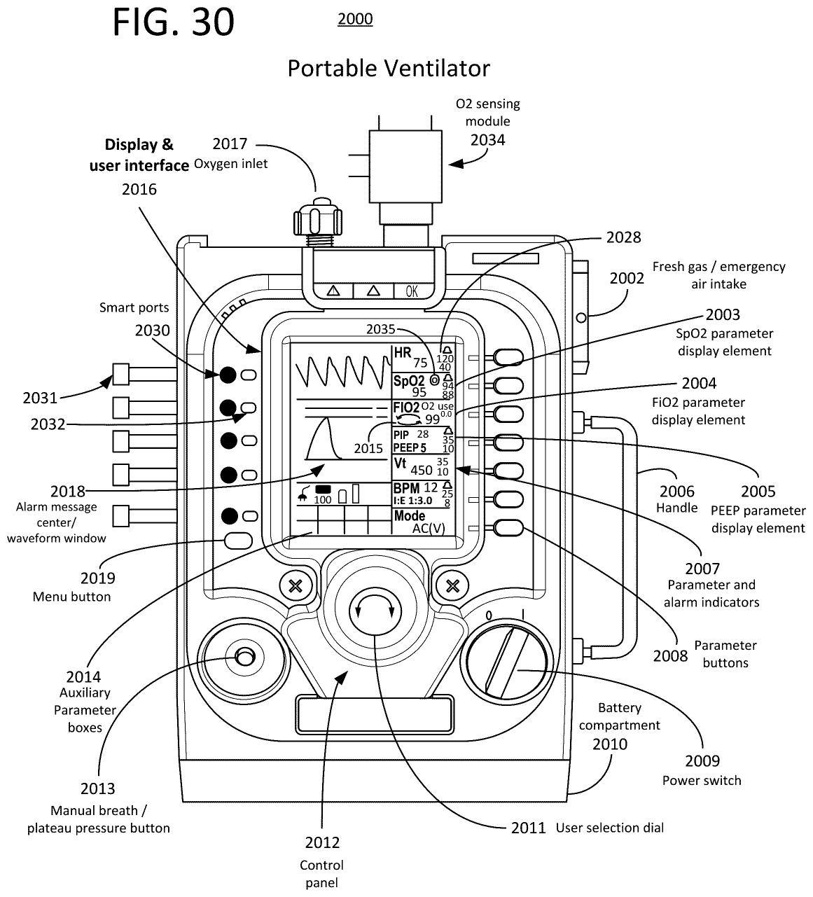

FIG. 30 is an illustration of a simplified example portable ventilator and display, with FIO2 closed loop control, which can be used in a hybrid ventilation system. As shown, the portable ventilator 2000 includes features such as a fresh gas/emergency air intake 2002, handle 2006, power switch 2009 (which, in other embodiments, could be, e.g., a soft button), battery compartment 2010, user selection dial 2011, control panel 2012, manual breath/plateau pressure button 2013, menu button 2019, oxygen inlet 2017, which may be used in coupling of an HPO2 source (or other pressurized oxygen source), and display and user interface 2016. In some embodiments, the handle 2006, and other features of the portable ventilator 2000, such as controls and display features and placement, are configured to enable single-handed operation of the portable ventilator 2000. In some such embodiments, for example, a user may grasp the handle of the ventilator 2000 with one or more fingers of a single hand (e.g., one or more of the index finger, middle finger, ring finger and pinkie) while simultaneously operating controls using one or more fingers of the single hand (e.g., one or more of the thumb, index finger and middle finger).

The fresh gas/emergency air intake 2002 provides a gas path and allows ambient air into the device's internal compressor. Built-in filters are used to protect the compressor and patient from particulate matter. The intake 2002 also acts as an anti-asphyxia valve that enables the patient to breathe ambient air, should the ventilator 2000 fail. The intake 2002 further contains a particulate filter and permits the user to connect either a bacteria/viral or a chemical/biological filter, depending on ambient conditions. Furthermore, an oxygen reservoir bag assembly may be connected to the intake 2002 to allow for low flow oxygen use with the ventilator 2000 in order to provide a source of supplemental oxygen to patients during ventilation. For example, low flow oxygen sources can be obtained based on a flow meter or an oxygen concentrator. Oxygen may be delivered through the intake 2002 when the ventilator's internal compressor cycles deliver breaths.

Additionally, in some embodiments, the ventilator 2000 may include smart ports 2030 and associated inserts/covers 2031, as described with reference to FIG. 1. Furthermore, indicators 2032, such as LED indicators, may be included in association with each of the smart ports 2030, and may, for example, illuminate to provide an indication when data, such as sensor data, is being communicated to the smart port 2030.

A top panel of the ventilator 2000 may have components including, in addition to the intake 2002 and the pulse oximeter connector 2001, a high-pressure oxygen input, a gas output, a power cord connector for external AC/DC power, a USB port, an exhalation valve, an exhaust valve and a transducer. A pulse oximeter, which, in various embodiments, may connect to the ventilator 2000 via one of the smart ports 203, or via another port or connector, may provide continuous non-invasive monitoring of SpO2 and pulse rate. Additionally, an oxygen sensing module 2034 including an O2 sensor, as described, e.g., with reference to FIG. 1, may couple with the inspiratory limb of the patient circuit connected to a port on the top panel of the ventilator 2000.

The portable ventilator 2000 may be operable using external AC/DC power or a battery, such as an internal lithium ion battery.

Furthermore, the ventilator 2000 may include at least one display and user interface 2016, which may, for example, include a liquid crystal display (LCD). Among other things, the display and user interface 2016 may provide a user with data relating to patient parameters and ventilation parameters, including current ventilator settings, which may be continuously updated. Furthermore, the display and user interface 2016 may include, among other things, various graphical user interface (GUI) aspects, allowing user interaction, such as to access particular data, change ventilator selections or settings, confirm suggested displayed changes to ventilator settings, receive and respond to alarms, etc. In particular, as shown, the display and user interface 2016 includes parameter and alarm indicators 2007, an alarm message center/waveform window 2018, parameter buttons 2008 and auxiliary parameter boxes 2014.

In some embodiments, the display and user interface 2016 may be divided into a number of sections. For example, as depicted, the top left area of the display and user interface 2016 may include airway pressure, flow, volume, capnography and plethysmography (pleth) waveform plots. This section may include displayed plots for airway pressure as well as, when a pulse oximeter is connected, the pleth waveform, and when a CO2 sensor is connected, the capnogram. When a plot is useful to facilitate a parameter adjustment by the user, a message area may display both the plot and a context menu that the user may use to make selections to obtain displayed context relating to the parameter.

The display and user interface 2016 also includes a menu display section in the top left area. This section may be used to display a menu after the user presses a menu button on the ventilator's control panel, and may be used to display context menus associated with particular parameters.

The display and user interface 2016 also includes an alarm message center/waveform window 2018 in the upper left area, in which visible alarms may at times be displayed. Some alarms may instruct the user to consult a physician, for example. In some embodiments, alarms may be categorized into different levels of priority, such as based on the level and/or urgency of the risk that the particular alarm condition may pose to the patient. Multiple alarms, along with their priorities, that have occurred recently may be available for display to a user, where the user may view the recent alarms by scrolling in a GUI, for example. In some embodiments, if the FIO2 setting is increased by a certain amount, such as 10% (or, e.g., 5-15%) during a predetermined period of time, such as 10 minutes (or, e.g., 5-15 minutes), an alarm is generated. Furthermore, in some embodiments, certain alarms may cause some or all CLC aspects, such as may include FIO2 CLC and/or PEEP CLC, to pause until the user clears the alarm. In some embodiments, during the time of the pause and before the alarm is cleared, the ventilator 2000 may operate using current parameter values, such as for FIO2 and PEEP, that were being used at the time that the pause was initiated.

The display and user interface 2016 may also include pop-up windows that may provide a user with context-sensitive guidance, such as in connection with manual adjustment of parameter values, for example.

The display and user interface 2016 also includes various parameter windows on the right side. Displayed parameters may include, e.g., SpO2, EtCO2, FIO2, PEEP, PIP, Vt, BPM and blood pressure, for example. Each parameter window may display a primary parameter as well as secondary parameters, such as parameters that may be related to the primary parameter or with associated alarm limits. In some embodiments, solid text may be displayed for primary and secondary parameter values that can be adjusted by the user, while outlined text may be used for patient-dependent parameters, for example. Primary parameters may also include mode, which may include a user selectable mode of operation including assist/control (AC), SIMV (synchronized Intermittent Mandatory Ventilation), continuous positive airway pressure (CPAP) and bilevel (BL).

Furthermore, the mode parameter may be associated with secondary parameter choices including volume targeting and pressure targeting. Volume targeting (V) assures that a constant volume is delivered to the patient in the inspiratory time using a constant flow. During volume targeting, the measured PIP parameter is displayed or highlighted. Pressure targeting (P) assures a constant airway pressure for the duration of the inspiratory time. During pressure targeting, the measured Vt parameter is displayed or highlighted.

The display and user interface 2016 also includes device-related icons section in the lower left area. This section may include icons that represent, and may provide status information on, for example, the ventilator's power source (which may indicate whether the ventilator is operating on external power or its battery), a battery charging status icon, an oxygen supply attachment icon, and may include an icon that indicates whether audible alarms or permitted or muted. Examples of particular icons are described with reference to FIG. 35.

The display and user interface 2016 may also include an auxiliary parameter boxes section 2014, which may be located toward the bottom. This section may display parameter boxes that allow the user to adjust a particular parameter using a context menu associated with the parameter.

In some embodiments, a user may take the following steps in setting up. The patient circuit may be attached to the ventilator's top panel. A high-pressure oxygen supply, if it is to be used, is attached. The user inspects the fresh gas/emergency air intake filters and may attach other items, such as an oxygen reservoir bag, and biological and chemical filters. The user may choose a power source, such as an external or internal power source. The user may connect the power supply to the ventilator. Once preliminary steps are completed, the user may power on the ventilator 2000 using the ventilator's power switch or button. Once powered on, the ventilator 2000 may perform a self-check, to check for potential alarm conditions as well as the operation of the pneumatic system, power system and internal communications system. During normal start-up, the ventilator's alarms may be muted for, e.g., 2 minutes, to allow the user to connect items including the patient circuit and pulse oximeter, and to perform operational tests.

In some embodiments, after powering on the ventilator 2000, the user may choose from the settings defaults, such as adult, pediatric, mask CPAP, custom (includes use of saved settings values), and last settings (includes use of last-used settings values). The user may select one of the defaults, in which case ventilation will be initiated using the default settings associated with the selection. Alternatively, the user may manually set settings using parameter buttons 2008. Furthermore, the user may select a mode of operation, including, as described briefly above, AC, SIMV, CPAP or BL. In AC, the patient receives either controlled or assisted breaths. When the patient triggers an assisted breath, the patient receives a breath, and either a pressure target or a volume target is utilized. In SIMV, the patient receives controlled breaths based on the setting of the breathing rate. In CPAP, the patient receives a constant positive airway pressure while breathing spontaneously. Spontaneous breaths may be either unsupported demand flow or supported using pressure support. In BL mode, the ventilator 2000 provides two pressure settings to assist the patient in breathing spontaneously, including a higher inspired positive airway pressure (IPAP) and a lower expiratory positive airway pressure (EPAP).

The parameter and alarm indicators 2007 may include information specifying current parameter values, and may also display other related information such as alarm threshold values 2028, which may indicate, for example, threshold beyond which an alarm may be triggered.

The parameter and alarm indicators 2007 may include an SpO2 parameter display aspect 2003, a FIO2 parameter display aspect 2004 and a PEEP parameter display aspect 2005.

In the SpO2 parameter display aspect 2003, the current patient SpO2 is displayed as “95”, meaning 95%. A target symbol 2035, along with the displayed numbers “94” and “88” indicate that the SpO2 target is set to 94% and the desaturation threshold of SpO2 is set to 88%.

In the FIO2 parameter display aspect 2004, the current FIO2 is displayed as “99”, meaning 99%. Double arrows 2015 (which, in some embodiments, may be animated as displayed), indicate that FIO2 closed loop control is currently turned on and operating. The displayed “O2 use” text indicates the current flow of high-pressure oxygen consumed with the current FIO2 setting.

In some embodiments, various alerts or warnings may be displayed to the user on the display and user interface 2016 before the user initiates FIO2 CLC. For example, the user may be warned not to use FIO2 CLC if the user suspects that pulse oximetry may not operate correctly or may not be available, or if the patient has carboxyhemoglobin poising (i.e., carbon monoxide poisoning), in which case the user may be advised to follow the local standard of care. The user may also be warned not to use FIO2 CLC for patients with a core temperature of less than 35 degrees Celsius. Furthermore, in some embodiments, pulse oximetry and a high-pressure oxygen source and/or POC may be required for FIO2 CLC, and failure of availability of either of these resources may cause FIO2 CLC to be paused and may cause an associated alarm to be displayed to the user.

In the PEEP parameter display aspect 2005, the current measured PIP is displayed as 28, meaning 28 cm H2O, and the current PEEP setting is displayed as 5, meaning 5 cm H2O. PIP alarm threshold levels are also displayed as 10 and 35, meaning 10 and 35 cm H2O.

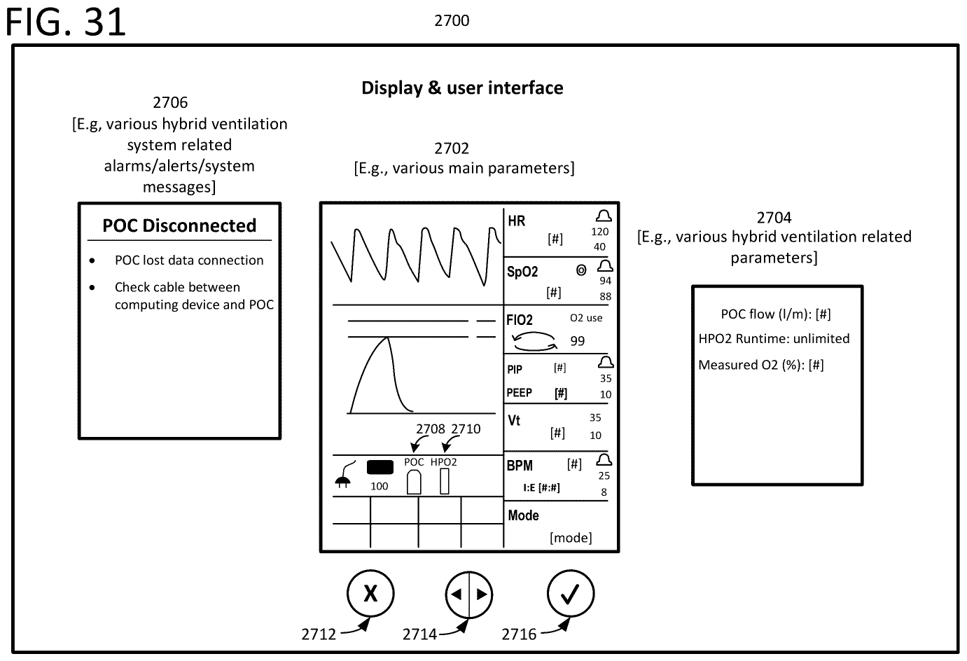

FIG. 31 is an illustration of a simplified example display and user interface 2700, which can be used in a hybrid ventilation system. It is noted that, in FIG. 30, the display and user interface 2016 is provided on a portable ventilator 2000. However, as described, for example, with reference to FIG. 1, in some embodiments, one or more ventilation related displays and user interfaces may be provided on one or more other devices or components of a distributed hybrid ventilation system, such as a CCM, defibrillator, portable computing device (e.g., tablet or smartphone), and/or non-local system or facility. Furthermore, in some embodiments, elements of a display and user interface, such as display and user interface 2700, may be shared or divided between devices and/or systems.

In the embodiment depicted in FIG. 31, the display and user interface 2700 includes a central or main portion 2702, as well as a left portion 2706 and a right portion 2704. The central or main portion 2702 includes parameter indicators including an indicator for the POC 2708 and an indicator for the HPO2 source 2710. These indicators 2708, 2710 may, for example, be LEDs that illuminate when the POC or HPO2 source are connected with the ventilator and/or are providing gas flow. As depicted, the display and user interface 2700 also includes displayed, user selectable selection buttons (whether physical or display-based), including a cancel/do not accept button 2712, a left and right scroll button 2714 and an accept button 2716. In some embodiments, the scroll button 2714 may be used to scroll between different displayed data (on, e.g., the central or main portion 2702, the left portion 2706, and the right portion 2704, or portions of any of them), and the accept button 2716 and cancel button 2712 may be used, respectively, to accept or reject, and/or to implement or not implement, e.g., displayed action options or suggestions.

As depicted, the left portion 2706 may be used for display (which may include user interface features, including touch screen) of, for example, various hybrid ventilation system related messaging, such as alarms, alerts, or system messages to a user, which may include guidance, instructions, recommendations, suggestions, selectable or implementable options, etc. For example, the messaging may include alerts or alarms relating to a POC or HPO2 source (or other pressurized oxygen source). As depicted in FIG. 31, the messaging of the left portion 2706 includes POC related messages, particularly, in this example, that the connection with the POC has been lost, and advising a user to check the cable between the computing device (to which the POC is connected for communication of data, such as a ventilator, CCM/defibrillator or portable computing device) and the POC. Other displayed alarms, alerts or system messages may relate, for example, to potential problems associated with the POC or HPO2 source (e.g., low POC battery), or to various aspects of system operation, e.g., that a particular FIO2 setting is not achievable using POC gas flow without HPO2 gas flow), or that the delivered FIO2 is too high or low (e.g., with the actual delivered FIO2 displayed as part of the alarm message), among other things.

As depicted, the right portion 2704 may be used for display of, for example, various hybrid ventilation related parameters, such as may include parameters associated with a POC or HPO2 source (or other pressurized oxygen source). For example, as depicted, the right portion 2704 includes displayed parameters including measured POC flow rate (in L/min), calculated/currently forecasted HPO2 source runtime (in, e.g., hours), and measured O2(%). For example, if an O2 sensor is coupled with the inspiratory limb of the patient circuit, then the measured O2 may relate to the oxygen concentration of gas being delivered to the patient, as described with reference to FIG. 1. Additionally, in some embodiments, displayed parameter values (e.g., of the right portion 2704 and potentially elsewhere) may update continuously and automatically, without requiring user action, and the displayed values may change as the parameters change (e.g., if the measured O2% is increasing, the displayed measured O2% will change accordingly, or if the projected HPO2 runtime changes, the displayed HPO2 runtime will change accordingly).

Various other displays and user interfaces, and associated user selection options and menus, may also be provided, whether on a ventilator or other device or system of a hybrid ventilation system. For example, the user of a device or system may be provided with options relating to turning on and off CLC of patient oxygenation, manually turning on or off a POC or HPO2 source, or manually setting a flow rate of the POC.

Some embodiments take into account, or correct for, differing gas densities, for example, to more accurately determine gas flow rates, as may affect, e.g., Qout, Qhp and Qpoc, as previously described. For example, in some embodiments, initial volumetric flow rate calculations and determinations may be based on an approximation, assumption or simplification that particular gases are of, or approximately of, the same density, or of the same density at sea level or a particular altitude. However, this may not always be the case, since, in fact, gas densities may vary (e.g., at sea level) depending on the composition of the gas, although typically in small proportion relative to each other. For example, since oxygen has a slightly greater density at sea level than ambient air, gases with an oxygen concentration greater than that of air may have a differing, such as a higher, density, than air. This can include, e.g., enriched oxygen gas from a POC (Qpoc) and gas flow through a compressor (Qc), where Qc may be a mixture of Qpoc and air flow (Qair). In some embodiments, calculations of flow from a pressurized oxygen source, such as an HPO2 source (Qhp), which does not flow through the compressor, are already calibrated to account for the density of the gas flow from the pressurized oxygen source (e.g., 100% oxygen or nearly 100% oxygen), and so no adjustment or correction may be required regarding Qhp. In some embodiments, by utilizing calculations or determinations (by the ventilator or other device) that take into account relevant differing gas densities, such as, e.g., between Qpoc, Qc and Qair, flow rates can be delivered that are more accurate, or corrected to be more accurate, by taking into account different densities and associated masses.

As such, in some embodiments, for some particular gas densities, differences may not be taken into account, such as based on the simplifying assumption that Qpoc, and Qc, are the same or approximately the same as Qair, given that gas density differences may be slight. However, in some embodiments, for particular gas densities, differences may be taken into account. In embodiments in which the particular differing gas densities are taken into account, either initial flow rate calculations may reflect the differences, or corrections may be applied after initial calculations, in order to account for the differing gas densities, for example.

The following is an example of calculation of a correction factor that can be applied to previously described calculations, to correct Qc (which may include contributions from Qair and Qpoc) for differing gas densities, assuming that the ventilator (or other device) is calibrated to initially calculate volumetric flow rates based on the simplifying assumption that the particular gases are of the same density, e.g., the density of air.

The density of gas may be calculated using the following equation:

$$ρ = \frac{M * P}{R * T} , \qquad ( Equation 13 )$$

where M is the molar mass, P is the atmospheric pressure, and R is the gas constant (R=8.3144598 m³·Pa·K⁻¹·mol⁻¹). The molar masses of air, oxygen, and argon are 28.97, 31.99, 39.978 g/mol, respectively. At standard temperature and pressure (STP, T=273.15 K, P=101,325 Pa), the air densities of air, oxygen, and argon are ρ_{Air}=1.2925, ρ_{O2}2=1.4277, and ρ_{Argon}=1.7823 kg/m³. The flow through the compressor comprises flow from the POC and flow drawn in through room air (Error! Reference source not found.). The POC gas comprises oxygen (typically FIO2,poc=93%) and other gases, which typically includes mostly argon:

Q_{C}=Q_{Air}+FIO_{2,POC}*Q_{POC}+(1−FIO_{2,POC})*Q_{POC} (Equation 14)

The combined FIO₂ of the gas mixture through the compressor is given by (noting that argon contains no O₂):

$${FI O}_{2 , C} = \frac{{FI O}_{2 , Air} * {Q}_{Air} * + {FI O}_{2 , POC} * {Q}_{POC}}{{Q}_{C}} \qquad ( Equation 15 )$$

The density of the gas mixture through the compressor is a function of the gas constituents:

$${ρ}_{C} = \frac{1}{{Q}_{C}} ( {ρ}_{A i r} * {Q}_{A i r} + {ρ}_{{O}_{2}} * {FI O}_{2 , POC} * {Q}_{P O C} + {ρ}_{A r g o n} * ( 1 - {FI O}_{2 , POC} ) * {Q}_{P O C} ) \qquad ( Equation 16 )$$

The mass flow rate through the compressor is calculated as the volumetric flow rate (Qc) times the gas density.

\dot{m}=ρQ_{C} (Equation 17)

Mass flow rate is typically expressed in units of kg/m³ whereas volumetric flow rate is in L/min. If only air is drawn in through the compressor, the mass flow rate through the compressor is:

\dot{m}_{Air}=ρ_{Air}Q_{C} (Equation 18)

whereas the mass flow rate for the actual gas mixture drawn through the compressor is:

\dot{m}_{C}=ρ_{C}Q_{C} (Equation 19)

Equating Equations 18 and 19, the correction factor to deliver the correct volumetric flow is the ratio of these two mass flow rates is:

$$k = \frac{{\dot{m}}_{C}}{{\dot{m}}_{Air}} = \frac{{ρ}_{C}}{{ρ}_{Air}} \qquad ( Equation 20 )$$

where ρ_{C} is calculated using Error! Reference source not found. and ρ_{Air}=1.2925 kg/m³.

In some embodiments, the controller of the compressor will use this factor to deliver the appropriate total volume to the patient:

Q′_{C}=k*Q_{C} (Equation 21)

where Q_{C}′ is the corrected compressor flow rate and Q_{C} is the uncorrected compressor flow rate. This correction factor can also be calculated as a linear function of FIO_{2,C}. For FIO_{2,C}=20.9%, the gas mixture is all air and k=1.

For FIO2,C=FIO2,POC, the gas mixture comprises only O2 and Argon and the correction factor is calculated as:

$$k = \frac{{ρ}_{C}}{{ρ}_{Air}} = \frac{{ρ}_{{O}_{2}} * {FI O}_{2 , POC} + {ρ}_{Argon} * ( 1 - {FI O}_{2 , POC} )}{{ρ}_{Air}} . \qquad ( Equation 22 )$$

Assuming FIO_{2,POC}=93%, the correction factor in this case equals 1.1238. Error! Reference source not found.

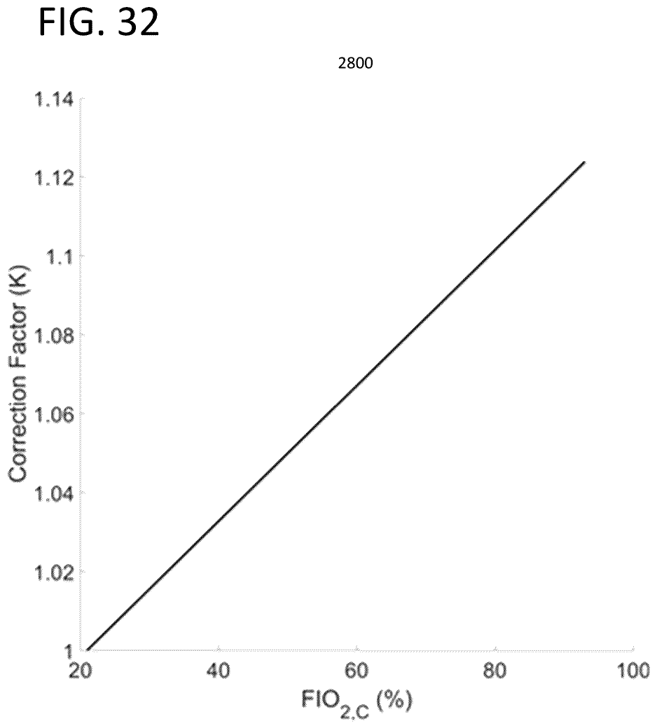

FIG. 32 illustrates a plot 2800 of the value of correction factor k, as given by Equation 22, as a function of FIO2,c, which is the FIO2 of gas through the compressor. As can be seen, the value of k varies linearly with the value of FIO2,c, such that, as FIO2,c increases, k increases linearly with it. This is logical given that, as FIO2,c increases, the oxygen concentration of the gas through the compressor increases, and, as a result, the density of the gas through the compressor increases, which leads to the correction factor increasing.

FIG. 33 illustrates example aspects of patient circuits 2500 that can be used in an example hybrid ventilation system including a portable ventilator 2514. Depicted components include an adult circuit 2512, including an inspiratory line 2502 and an expiratory line 2506, and an infant/pediatric circuit 2510, including an inspiratory line 2504 and an expiratory line 2508.

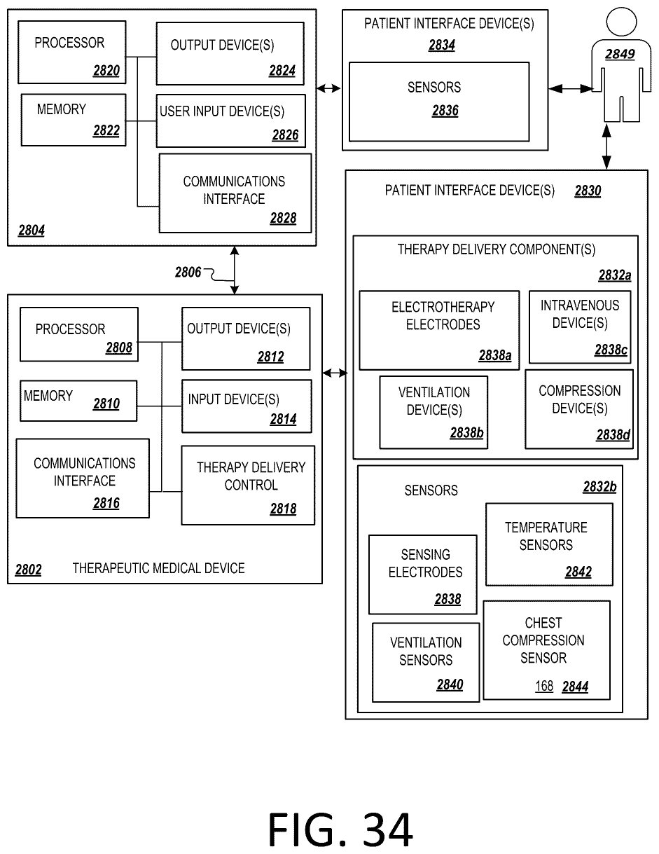

FIG. 34 illustrates an example of components of various devices described with reference to prior figures. The components 2808, 2810, 2812, 2814, 2816, and 2818 are communicatively coupled (directly and/or indirectly) to each other for bi-directional communication. Similarly, the components 2820, 2822, 2824, 2826, and 2828 are communicatively coupled (directly and/or indirectly) to each other for bi-directional communication.

In some implementations, the components 2808, 2810, 2816, and/or 2818 of the therapeutic medical device 2802 may be combined into one or more discrete components and components 2816 and/or 2818 may be part of the processor 2808. The processor 2808 and the memory 2810 may include and/or be coupled to associated circuitry in order to perform the functions described herein. Additionally, the components 2820, 2822, and 2828 of companion device 2804 may be combined into one or more discrete components and component 2828 may be part of the processor 2820. The processor 2820 and the memory 2822 may include and/or be coupled to associated circuitry in order to perform the functions described herein.

In some implementations, the therapeutic medical device 2802 may include the therapy delivery control module 2818. For example, the therapy delivery control module 2818 may be an electrotherapy delivery circuit that includes one or more high-voltage capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2818 may be a compression device electro-mechanical controller configured to control a mechanical compression device. As a further example, the therapy delivery control module 2818 may be an electro-mechanical controller configured to control drug delivery, temperature management, ventilation, and/or other type of therapy delivery.

The therapeutic medical device 2802 may incorporate and/or be configured to couple to one or more patient interface devices 2830 and patient interface devices that may be coupled with a patient 2849. The patient interface devices 2830 may include one or more therapy delivery component(s) 2832a and one or more sensor(s) 2832b. Similarly, the companion device 2804 may be adapted for medical use and may incorporate and/or be configured to couple to one or more patient interface device(s) 2834. The patient interface device(s) 2834 may include one or more sensors 2836. The sensor(s) 2836 may be substantially as described herein with regard to the sensor(s) 2832b.

The sensor(s) 2832b and 2836 may include sensing electrodes (e.g., the sensing electrodes 2838), ventilation and/or respiration sensors (e.g., the ventilation and/or respiration sensors 2830), temperature sensors (e.g., the temperature sensor 2842), chest compression sensors (e.g., the chest compression sensor 2844), etc. In some implementations, the information obtained from the sensors 2832b and 2836 can be used to generate information displayed at the therapeutic medical device 2802 and simultaneously at the display views at companion device 2804 and described above. In one example, the sensing electrodes 2838 may include cardiac sensing electrodes. The cardiac sensing electrodes may be conductive and/or capacitive electrodes configured to measure changes in a patient's electrophysiology to measure the patient's ECG information. The sensing electrodes 2838 may further measure the transthoracic impedance and/or a heart rate of the patient. The ventilation and/or respiration sensors 2830 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, impedance sensors, and combinations thereof. The temperature sensors 2842 may include an infrared thermometer, a contact thermometer, a remote thermometer, a liquid crystal thermometer, a thermocouple, a thermistor, etc. and may measure patient temperature internally and/or externally. The chest compression sensor 2844 may include one or more motion sensors including, for example, one or more accelerometers, one or more force sensors, one or more magnetic sensors, one or more velocity sensors, one or more displacement sensors, etc. The chest compression sensor 2844 may provide one or more signals indicative of the chest motion to the therapeutic medical device 2802 via a wired and/or wireless connection. The chest compression sensor 2844 may be, for example, but not limited to, a compression puck, a smart-phone, a hand-held device, a wearable device, etc. The chest compression sensor 2844 may be configured to detect chest motion imparted by a rescuer and/or an automated chest compression device (e.g., a belt system, a piston system, etc.). The chest compression sensor 2844 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2844.

In various implementations, the sensors 2832b and 2836 may include one or more sensor devices configured to provide sensor data that includes, for example, but not limited to ECG, blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2832a may include electrotherapy electrodes (e.g., the electrotherapy electrodes 2838a), ventilation device(s) (e.g., the ventilation devices 2838b), intravenous device(s) (e.g., the intravenous devices 2838c), compression device(s) (e.g., the compression devices 2838d), etc. For example, the electrotherapy electrodes 2838a may include defibrillation electrodes, pacing electrodes, and combinations thereof. The ventilation devices 2838b may include a tube, a mask, an abdominal and/or chest compressor (e.g., a belt, a cuirass, etc.), etc. and combinations thereof. The intravenous devices 2838c may include drug delivery devices, fluid delivery devices, and combinations thereof. The compression devices 2838d may include mechanical compression devices such as abdominal compressors, chest compressors, belts, pistons, and combinations thereof. In various implementation, the therapy delivery component(s) 2832a may be configured to provide sensor data and/or be coupled to and/or incorporate sensors. For example, the electrotherapy electrodes 2838a may provide sensor data such as transthoracic impedance, ECG, heart rate, etc. Further the electrotherapy electrodes 2838a may include and or be coupled to a chest compression sensor.

As another example, the ventilation devices 2838b may be coupled to and/or incorporate flow sensors, gas species sensors (e.g., oxygen sensor, carbon dioxide sensor, etc.), etc. As a further example, the intravenous devices 2838c may be coupled to and/or incorporate temperature sensors, flow sensors, blood pressure sensors, etc. As yet another example, the compression devices 2838d may be coupled to and/or incorporate chest compression sensors, patient position sensors, etc. The therapy delivery control modules 2818 may be configured to couple to and control the therapy delivery component(s) 2832a, respectively.

The one or more sensor(s) 2832b and 2836 and/or the therapy delivery component(s) 2832a may provide sensor data. The patient data provided at the display screens of the therapeutic medical device 2802 and companion device 2804 may display the sensor data. For example, the therapeutic medical device 2802 may process signals received from the sensor(s) 2832b and/or the therapy delivery component(s) 2832a to determine the sensor data. Similarly, the companion device 2804 may process signals received from the sensor(s) 2836 and/or sensor data from the sensors 2832b received via the therapeutic medical device 2802 to determine the sensor data.

Some embodiments include providing mechanical ventilation in connection with, for example, a patient experiencing respiratory distress. Respiratory distress may include, for example, any form of respiratory or breathing difficulty, impairment or problem, including respiratory failure. Respiratory parameter data can include data relating to respiratory, pulmonary or lung related parameters, as may, for example, be associated with respiratory, pulmonary or lung related characteristics, attributes or functions. Respiratory parameter data can include, for example, data related to pulmonary associated pressure, flow, volume or capacity related parameters, including parameters that may be measured using one or more flow sensors, pneumotachometers or spirometers. Respiratory parameter data can include, for example, data relating to respiratory mechanics, such as respiratory compliance (Crs), respiratory elastance (E) and respiratory resistance (Rrs). Respiratory parameter data can also include, for example, parameters such as vital capacity (VC), forced vital capacity (FVC), forced expiratory volume (FEV) at timed intervals (e.g., between 0.5 and 10 seconds, less than 0.5 second, 0.5 second, 1.0 second or FEV1, 2.0, 3.0 seconds, 4.0 seconds or 5.0 seconds), forced expiratory flow (FEF) at, e.g., 10%-90% capacity, such as 25%-75% or FEF25-75, peak expiratory flow rate (PEF or PEFR) and maximum breathing capacity (sometimes called maximal voluntary ventilation or MVV). Respiratory parameter data may be presented in various different ways or forms, such as may include, for example, in raw terms, such as liters or liters per second, or as percentages. Respiratory parameter data may also be presented, for example, as “predicted” values, such as percent predicted, which can, for example, include results as a percentage value in connection with a reference, average, or other “predicted” value. “Predicted” values may, in some cases, be associated with patients or hypothetical patients of one or more similar characteristics.

A respiratory status may, for example, relate to, identify, or indicate the presence or absence of, one or more respiratory, pulmonary or lung related conditions, and may include respiratory distress. A condition may include a state, disease, or problem, or several thereof, or a type, group or category thereof. A respiratory status may include an etiology relating to a respiratory condition or a non-respiratory condition (e.g., respiratory distress associated with acute heart failure). A non-respiratory condition may, for example, be associated with one or more non-respiratory systems, e.g., cardiac, endocrine, exocrine, circulatory, immune, lymphatic, nervous, muscular, renal, skeletal, or others. Additionally, a respiratory status may include one or more determined, assessed, estimated, probable or possible conditions, or determined, assessed, probable or possible associated etiologies. A respiratory status may also include data associated any of the foregoing, which may be called respiratory status data.

Ventilators, such as portable ventilators, and ventilation systems, may include, for example, devices or systems capable of delivering ventilation, whether such delivered ventilation is controlled internally, remotely, or with aspects of both.

Various ventilation parameter related terms or abbreviations, including fraction of inspired oxygen (FIO2), positive end-expiratory pressure (PEEP) and others, refer to ventilation related settings, even though the word “setting” may or may not be stated. Furthermore, reference to a ventilation parameter, parameter setting, or setting may be used to refer to the parameter in a conceptual or definitional sense, or the value associated with a particular setting. A user may include an individual operating, supervising or in whole or in part responsible for operation of a device such as a portable ventilator, even if, during a particular period of time while the device is operating, the user may not be interacting with the device.

Ventilation related settings may include, for example, any setting, such as a current, selected, set or entered ventilation parameter, parameter value, or other setting relating to ventilation, any aspect of ventilation, operation of a ventilator, such as a portable ventilator, in association with providing or provided ventilation, including mechanical ventilation. Ventilation related settings may include, for example, settings related to a mode or form of ventilation, power, communication, network connection, remote or internal control, ventilation parameters such as FIO2, PEEP (or baseline airway pressure (BAP)), closed loop control (CLC), etc., among other things.

An alert or alarm may be presented for the attention of a user, such as by being visually or audibly presented, such as via a display, graphical user interface (GUI) or speaker of a device. However, an alert or alarm may also include an alert or alarm condition that is algorithmically identified, recognized or determined by a computerized device, and not necessarily presented or displayed. The term optimizing may include, for example, improvement or improved operation in one or more aspects, for example, relative to an actual, potential or hypothetical less optimized situation or less optimized operation. The term adjusting can include changing as well as not changing or maintaining without change, as may be appropriate. A determined parameter value can include a determined estimated or determined approximated value for the parameter. The term monitoring can refer to or include, for example, monitoring or tracking performed by a computerized device utilizing one or more algorithms and not by a person or user, or monitoring by a person or user, or both. The term continuous can include, among other things, on a periodic basis (with identical or different periods), on a frequent basis, on a repeated basis, or cyclically, for example.

In some embodiments, a controller of a ventilator internally signals a mechanical ventilation system (or apparatus) of the ventilator in order to implement control thereof, whether such internal signaling implements internal control or remote control. Internal control includes a controller of a portable ventilator internally signaling the mechanical ventilation system of the portable ventilator to implement control according to a setting value (or more than one setting value) that the controller determines, even if the determined setting value is determined based at least in part on a setting value of a received request/command. Remote control includes the controller of the portable ventilator internally signaling the mechanical ventilation system of the portable ventilator according to a setting value received by the controller via a request/command from a remote control device or system.

In some embodiments, a ventilator may be capable of either internal or remote control, or operation with aspects of both, and may be capable of determining which, or what particular state or mode, is employed or used at a predetermined or particular time. Furthermore, a portable ventilator may be capable of switching between modes or states of operation, such as may include an internal control mode, a remote control mode, or a mode that includes aspects of both.

A controller can include physical aspects such as one or more processors and one or more memories, as well as software based aspects, which may include one or more programs, algorithms or software based aspects. The software based aspects may, for example, be stored, in the one or more memories of the controller, accessed by the one or more processors of the controller and used or executed by the processor.

The term controls may include physical controls, display/GUI based controls, or both. Controls may, for example, allow user interaction with a device or system, such as may affect operation of the device or system, which may include, for example, making selections or providing input to set, select, change, increase, decrease, confirm or override implementation of a parameter setting value or a mode. A device or system with controls may, in various aspects, operate without user interaction, with user interaction, or with aspects of both. This may include operation that may proceed, in various aspects or instances, without user interaction, but may permit of, or require, user interaction in some aspects or instances. A display/GUI may include controls, and/or controls may include a display/GUI.

In some embodiments, a set of controls, such as of a particular device, may include one or more respiratory aspects. Respiratory aspects of controls may include, for example, display/GUI aspects relating to any aspects of ventilation, ventilation parameters or aspects relating to respiration, respiratory distress or respiratory parameters.

Herein, remote control may include control, or aspects of control, provided from outside of, or separately from, a controlled device or system, such as by wired or wireless signaling or communication, such as may include, for example, Bluetooth or ultrawide band. As such, remote control may be implemented via a wired only connection that connects a remote control device and a remotely controlled device, or by a wireless only connection, or by a combination of wired connection and wireless connection, for example. Remote control, such as remote control by a remote control device or system, may include control with or without aspects of user interaction with the remote control device or system, such as, for example, by the user using controls of the remote control device or system. Internal control, such as a device internally controlling itself, may or may not include aspects of user interaction with the internally controlled device, such as, for example, by the user using controls of the internally controlled device. User interaction may include, for example, a user using controls to input, set, select, change, increase, decrease, confirm or override implementation of a parameter setting value or a mode. User interaction may or may not be prompted, guided, or interactively guided, e.g., via one or more GUI based prompt messages, alarms or alerts that may relate, for example, to patient parameters or ventilation parameters, such as may relate to ventilator operation, performance or components, such as sensors.

Any of various types of gas movers/gas flow generators may be used in various embodiments of a portable ventilator, including blowers, compressors, compressor-based and turbomachinery. In some embodiments, centrifugal blowers are included (or used). Herein, an electronic circuit board may include a printed circuit board (PCB) or boards.

In some embodiments, a ventilator, such as a portable ventilator, is configured for integration with one or more other devices or systems, such as onsite or offsite remote devices, systems or interfaces, aspects of which may or may not be included in various embodiments. For example, in some embodiments, the ventilator may couple, in a wired and/or wireless fashion, with a device such as a patient monitor or critical care monitor (CCM). In some embodiments, for example, a defibrillator, portable ventilator, or other device, such as another medical device, monitoring device or computing device, may be, include or function as a CCM. Furthermore, in some embodiments, the ventilator may couple with one or more computing systems, computers, computing devices or portable computers, which can include, for example, a cloud-basing computing system, computer, notebook computer, tablet, touch-based device, smartphone, wearable device or implantable device, among other things.

In some embodiments, smaller size, smaller footprint, lighter weight and/or greater simplicity may be desirable or optimized in a portable ventilator of a hybrid ventilation system. Optimization of a hybrid ventilation system or components thereof, or other devices in the environment thereof, such as a defibrillator or tablet, may take into account various factors such as the actual, predicted, likely or anticipated setting, context, environment or application, such as may include non-hospital contexts. Optimization may also take into account allocation of roles of various devices in such contexts. Furthermore, the hybrid ventilation system or components thereof may be optimized for users with limited training or experience. Still further, the hybrid ventilation system or components thereof may be optimized for use in environments that may include other integrated devices or systems.

In some embodiments, a portable ventilator of a hybrid ventilation system may be capable of obtaining patient respiratory parameter data, which can be used in determining a respiratory status of the patient, and may be used in determining a disease state of etiology of the patient, and also in determining appropriate patient treatment. In various embodiments, such determinations may be made, for example, by the portable ventilator itself or by another device to which the portable ventilator sends data, such as respiratory parameter or respiratory status data. This capability of the portable ventilator may be especially advantageous in various non-hospital and crisis settings, in which availability of other devices may be limited or unpredictable.

Furthermore, in some embodiments, a portable ventilator of a hybrid ventilation system may be capable of either internal control, remote control, or both. In some embodiments, this, in turn, may allow use of a portable ventilator that has less internal controls and/or is smaller or lighter, which can also be especially advantageous in various non-hospital and crisis settings.

For example, the space immediately surrounding a critical care patient can be very crowded with equipment or items such as, for example, hoses, tubes, wires and various devices. It can be beneficial or advantageous to minimize, for example, the spatial impact of a ventilator. In some embodiments, for example, a portable ventilator may provide advantages by allowing modularization of, or allowing a higher degree of modularization of, a system including a ventilator, which may allow for a much smaller, simpler ventilator close to the patient. Furthermore, in some embodiments, the controls in or of a remote control device or system may be some variable distance from the patient (e.g. between 0-3 feet, between 3-6 feet, between 6-9 feet, between 9-12 feet, further than 12 feet, or offsite). Some embodiments provide benefits or advantages to patient care by, for example, simplifying the immediate clinical environment of the patient. For example, in some embodiments, even if the combined weight and/or size of a combination of a ventilator and different remote control device, together, are the same or similar to a system in which the controls are provided in or with the ventilator, the combination of the ventilator and the different remote control device may be more usable, or more practically or optimally useable, in various clinical contexts, for example, since only a relatively small ventilator may be required to be at the patient's side or very close to the patient. Some embodiments provide a portable ventilator, of a hybrid ventilation system, that provides these advantages.

In some embodiments, an overall environment may include a portable ventilator or hybrid ventilation system that may couple and operationally integrate with one or more other devices, each of which may provide certain roles or functionality in the overall environment. These roles may relate to, for example, device and patient related sensing, operation, processing, software, algorithms, applications, data storage, communication, integration (of devices and systems, roles, functions, and physical, software based and conceptual components and aspects), user interaction and guidance, and patient related assessments or interventions. Within the integrated environment, roles of each device may be optimized, taking into account, for example, factors relating to each device or system as well as the overall environment and anticipated settings. Such optimization may also take into account factors relating to the need for the hybrid ventilation system or components thereof to be of a particular or sufficiently small size, light weight, and/or degree of simplicity or ease of portability or use. In some embodiments, a hybrid ventilation system augments the ability to accurately assess or screen a patient's condition, to determine or select, and initiate and deliver, an effective intervention, and/or to ongoingly control or adjust, or assist in control and adjustment of, parameters relating to an applied intervention. Furthermore, this augmentation may be optimized in view of the overall environment, available devices, systems or users at a given time or time period, and available communication between them at a given time or time period, as well as changing aspects thereof over time.

In some embodiments, for example, one or more devices or systems coupled with a portable ventilator, or integrated into the environment thereof, may supply, or partially supply, components or other aspects that may augment or enhance the operation of the portable ventilator, and/or otherwise might be, or need to be, included with or in the portable ventilator. Some such aspects may include physiological and operational signaling, sensing or measuring aspects. Other aspects may relate to power, communication, device recognition, authentication or coupling. Other aspects may relate to processing, hardware and software, such as for data processing and management, data and device integration, or operational aspects such as patient oxygenation or other ventilation related parameters or settings. Other aspects may related to patient respiratory or status assessment, diagnostic screening, diagnosis, treatment or intervention. Other aspects may include controls and control relating to the portable ventilator (physical, display/GUI based, or both), as well as features thereof.

In some embodiments, a portable ventilator may have a set of (one or more) included controls, while one or more remote control devices, systems or monitors may also have a set of (one or more) remote controls relating to the portable ventilator. A set of remote controls may for allow remote control of the portable ventilator, such as may relate, for example, to aspects of operation of a mechanical ventilation system of the portable ventilator. In various embodiments, a set of included controls of portable ventilator may vary across a spectrum of possibilities.

In various embodiments, a set of remote controls may include respiratory aspects that are separate from other aspects, or may include respiratory aspects that are mixed, combined or integrated, for example, into a larger display/GUI or set of controls that may include non-respiratory aspects as well. Furthermore, in various embodiments, a set of remote controls may be separate or separable from other aspects of controls, or may be integrated with other aspects. In some embodiments, the portable ventilator may have no included controls at all, or may have a limited set of included controls. For example, any or many controls relating to the portable ventilator may be provided by one or more sets of remote controls of one or more other devices, systems or monitors. In some embodiments, a set of remote controls, or a portion thereof, may, in whole or in part, correspond to, emulate, mirror, duplicate, replicate, simulate, or be in any of varying degrees similar to a set of included controls of the portable ventilator. Furthermore, in some cases in which the portable ventilator has no controls or a limited set of controls, a set of remote controls may, to some degree, be similar to or replicate a hypothetical set of included controls that might be included in a portable ventilator.

The description set forth herein in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “substantially”, “about,” “proximate,” “minor variation,” and similar terms generally refer to ranges that include the identified value within a margin of 20%, 10%, 5%, or less than 5%, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In some instances, variations of a term may be utilized that may refer to the same or similar concepts, and certain terms may have meanings that are informed by a particular context. Generally, sending, receiving, or transmitting of data may include by wired and/or wireless connection, and/or within one or more wired or wireless networks. Furthermore, sending from a first entity to a second entity, or to be received by the second entity, can include sending from the first entity to the second entity, or to be received by the second entity, directly from the first entity to the second entity, or indirectly via one or more intermediary entities.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1-288. (canceled)

289. An apparatus for providing mechanical ventilation to a patient, comprising:
a gas delivery apparatus, having a patient interface, configured for gas delivery to the patient;
at least one oxygen concentrator, coupled with the gas delivery apparatus, for generating oxygen enriched gas for delivery to the patient via the gas delivery apparatus;
at least one pressurized oxygen source, coupled with the gas delivery apparatus, for providing oxygen from the at least one pressurized oxygen source for delivery to the patient via the gas delivery apparatus; and
a controller, in communication with the gas delivery apparatus, for causing gas to be delivered to the patient in accordance with a FIO2 setting, the controller being configured to:
based at least in part on the FIO2 setting, determine an oxygen enriched gas flow rate of the oxygen enriched gas for the gas to be delivered to the patient and a pressurized oxygen source flow rate of the oxygen from the at least one pressurized oxygen source for the gas to be delivered to the patient, and
control the gas delivery apparatus to deliver the gas to the patient in accordance with the FIO2 setting, the determined oxygen enriched gas flow rate, and the determined pressurized oxygen source flow rate.

290. The apparatus of claim 289, wherein the determination of the oxygen enriched gas flow rate and the pressurized oxygen source flow rate comprises preferentially using the oxygen enriched gas flow rate relative to the pressurized oxygen source flow rate.

291. The apparatus of claim 290, wherein the determination of the oxygen enriched gas flow rate and the pressurized oxygen source flow rate comprises using a maximum available oxygen enriched gas flow rate for the oxygen enriched gas flow rate, and supplementing the maximum available oxygen enriched gas flow rate using the pressurized oxygen source flow rate as necessary to achieve the FIO2 setting.

292. The apparatus of claim 289, comprising:
at least one oximetry sensor configured to generate signals representative of an oxygen concentration of the patient's blood,
wherein the controller is configured to:
based at least in part on the signals representative of the oxygen concentration of the patient's blood, determine the FIO2 setting for the gas to be delivered to the patient.

293. The apparatus of claim 292, wherein the controller is configured to:
receive the signals representative of the oxygen concentration of the patient's blood from the at least one oximetry sensor during the delivery of the gas to the patient,
based at least in part on the received signals, determine the oxygen concentration of the patient's blood, and
based at least in part on the oxygen concentration of the patient's blood, determine the FIO2 setting.

294. The apparatus of claim 293, wherein the controller is configured to adjust the FIO2 setting to maintain patient oxygenation at a desired level or range, and wherein the controller is configured to utilize closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at the desired level or range.

295. The apparatus of claim 294, wherein the minimum available oxygen enriched gas flow rate is between 0.1 and 1.0 L/min and the maximum available oxygen enriched gas flow rate is between 2.5 and 3.5 L/min.

296. The apparatus of claim 289, wherein, when the oxygen enriched gas flow rate is at a maximum available oxygen enriched gas flow rate, the determination of the pressurized oxygen source flow rate is based at least in part on the FIO2 setting, an oxygen concentration of the oxygen enriched gas, an overall flow rate of the gas to be delivered to the patient, and the oxygen enriched gas flow rate.

297. The apparatus of claim 296, wherein the FIO2 setting is adjustable, and wherein the controller is configured to, based at least in part on adjustments to the FIO2 setting, make adjustments to the oxygen enriched gas flow rate and the pressurized oxygen source flow rate.

298. The apparatus of claim 289, wherein the oxygen enriched gas has an oxygen concentration of between 90% and 96%.

299. The apparatus of claim 298, wherein the oxygen enriched gas has an oxygen concentration of 93%.

300. The apparatus of claim 289, wherein the at least one oxygen concentrator comprises at least two oxygen concentrators.

301. The apparatus of claim 300, wherein the at least two oxygen concentrators comprises a first oxygen concentrator and a second oxygen concentrator, and wherein determining the gas flow rate of the oxygen enriched gas comprises determining a first gas flow rate for oxygen enriched gas from the first oxygen concentrator and a second gas flow rate for oxygen enriched gas from the second oxygen concentrator.

302. The apparatus of claim 289, wherein determining the oxygen enriched gas flow rate comprises calculating and taking into account a difference between the density of the oxygen enriched gas and a density of air.

303. A system for providing mechanical ventilation to a patient, comprising:
a gas delivery system, having a patient interface, configured for gas delivery to the patient;
at least one oxygen concentrator, coupled with the gas delivery system, for generating oxygen enriched gas for delivery to the patient via the gas delivery system;
at least one pressurized oxygen source, coupled with the gas delivery system, for providing oxygen from the at least one pressurized oxygen source for delivery to the patient via the gas delivery system; and
a controller for causing gas to be delivered to the patient in accordance with a FIO2 setting, the controller being configured to:
based at least in part on the FIO2 setting, determine an amount of oxygen supplementation for the gas to be delivered to the patient, comprising:
when the FIO2 setting can be achieved with use of only the oxygen enriched gas for the oxygen supplementation, using, for the oxygen supplementation, only the oxygen enriched gas,
when the FIO2 setting cannot be achieved with use of only oxygen enriched gas for the oxygen supplementation, using, for the oxygen supplementation, the oxygen enriched gas and the oxygen from the at least one pressurized oxygen source, and
control the gas delivery system to deliver the gas to the patient in accordance with the FIO2 setting and the determined amount of oxygen supplementation.

304. The system of claim 303, wherein the determination of the amount of oxygen supplementation comprises preferentially using the oxygen enriched gas for the oxygen supplementation relative to the oxygen from the at least one pressurized oxygen source.

305. The system of claim 304, wherein the determination of the amount of oxygen supplementation comprises using a maximum available amount of the oxygen enriched gas for the oxygen supplementation, and supplementing the maximum available amount of the oxygen enriched gas using the pressurized oxygen from the at least one pressurized oxygen source as necessary to achieve the FIO2 setting.

306. The system of claim 303, wherein the oxygen supplementation supplements any oxygen provided by any ambient air included in the gas being delivered to the patient.

307. The system of claim 303, wherein the controller is configured to cause the gas to be delivered to the patient in accordance with the FIO2 setting, the determined amount of oxygen supplementation, and an overall flow rate for the gas to be delivered to the patient.

308. The system of claim 307, wherein the amount of oxygen supplementation comprises a flow rate of the oxygen enriched gas and a flow rate of the oxygen from the at least one pressurized oxygen source.

309. The system of claim 308, wherein the overall flow rate comprises a flow rate of ambient air.

310. The system of claim 303, wherein the at least one oxygen concentrator comprises at least two oxygen concentrators.

311. The system of claim 310, wherein the at least two oxygen concentrators comprise a first oxygen concentrator and second oxygen concentrator, and wherein using, for the oxygen supplementation, the oxygen enriched gas comprises using, for the oxygen supplementation, oxygen enriched gas from the first oxygen concentrator and oxygen enriched gas from the second oxygen concentrator.

312. The system of claim 303, comprising:
at least one oximetry sensor configured to generate signals representative of an oxygen concentration of the patient's blood,
wherein the controller is configured to:
based at least in part on the signals representative of the oxygen concentration of the patient's blood, determine the FIO2 setting for the gas to be delivered to the patient.

313. The system of claim 312, wherein the controller is configured to:
receive the signals representative of the oxygen concentration of the patient's blood from the at least one oximetry sensor during the delivery of the gas to the patient,
based at least in part on the received signals, determine the oxygen concentration of the patient's blood, and
based at least in part on the oxygen concentration of the patient's blood, determine the FIO2 setting.

314. The system of claim 313, wherein the controller is configured to adjust the FIO2 setting to maintain patient oxygenation at a desired level or range.

315. A method for controlling mechanical ventilation being provided to a patient, comprising:
a controller controlling a gas delivery system, of a mechanical ventilation system, to deliver gas to the patient, comprising:
based at least in part on signals representative of an oxygen concentration of the patient's blood obtained from at least one oximetry sensor, determining a FIO2 setting for the gas to be delivered to the patient; and
causing the gas to be delivered to the patient in accordance with the FIO2 setting, comprising:
based at least in part on the FIO2 setting, determining an amount of oxygen supplementation for the gas to be delivered to the patient, comprising determining an oxygen concentrator system contribution, of oxygen enriched gas from an oxygen concentrator system, to the amount of oxygen supplementation, and determining a pressurized oxygen source contribution, of oxygen from at least one pressurized oxygen source, to the amount of oxygen supplementation, and
controlling the gas delivery system to deliver the gas to the patient in accordance with the FIO2 setting and the determined amount of oxygen supplementation.

316. The method of claim 315, wherein the oxygen concentrator system comprises at least two oxygen concentrators, comprising a first oxygen concentrator and a second oxygen concentrator, and wherein determining the oxygen concentrator system contribution comprises determining a first oxygen concentrator contribution from the first oxygen concentrator and a second oxygen concentrator contribution from the second oxygen concentrator.

317. The method of claim 315, comprising the controller utilizing closed loop control based on oxygen concentration measurements of the patient's blood, comprising utilizing the at least one oximetry sensor to monitor the oxygen concentration of the patient's blood and adjusting oxygen delivery during the delivery of the gas to the patient to maintain the oxygen concentration of the patient's blood at a desired level or range.

318. The method of claim 315, wherein the oxygen concentrator system comprises at least two oxygen concentrators comprising a first oxygen concentrator and a second concentrator, and wherein determining the oxygen concentrator system contribution comprises determining a contribution from the first oxygen concentrator and a contribution from the second oxygen concentrator.

319. An apparatus for providing mechanical ventilation to a patient, comprising:
a gas delivery apparatus, having a patient interface, configured for gas delivery to the patient;
at least one oxygen concentrator, coupled with the gas delivery apparatus, for generating oxygen enriched gas for delivery to the patient via the gas delivery apparatus; and
a controller, in communication with the gas delivery apparatus, for causing gas to be delivered to the patient in accordance with a FIO2 setting, the controller being configured to:
based at least in part on the FIO2 setting, determine an oxygen enriched gas flow rate of the oxygen enriched gas for the gas to be delivered to the patient, and
control the gas delivery apparatus to deliver the gas to the patient in accordance with the FIO2 setting and the determined oxygen enriched gas flow rate.

320. The apparatus of claim 319, comprising at least one pressurized oxygen source, coupled with the gas delivery apparatus, for providing oxygen from the at least one pressurized oxygen source for delivery to the patient via the gas delivery apparatus, and wherein the controller is configured to:
based at least in part on the FIO2 setting, determine the oxygen enriched gas flow rate of the oxygen enriched gas for the gas to be delivered to the patient and a pressurized oxygen source flow rate of the oxygen from the at least one pressurized oxygen source for the gas to be delivered to the patient, and
control the gas delivery apparatus to deliver the gas to the patient in accordance with the FIO2 setting, the determined oxygen enriched gas flow rate, and the determined pressurized oxygen source flow rate.

321. The apparatus of claim 319, wherein the controller is configured to adjust the FIO2 setting to maintain patient oxygenation at a desired level or range, and wherein the controller is configured to utilize closed loop control based on oxygen concentration measurements of the patient's blood, comprising utilizing at least one oximetry sensor to monitor the oxygen concentration of the patient's blood and adjusting oxygen delivery during the delivery of the gas to the patient to maintain the oxygen concentration of the patient's blood at the desired level or range.

322. The apparatus of claim 319, wherein the at least one oxygen concentrator comprises at least two oxygen concentrators comprising a first oxygen concentrator and a second oxygen concentrator, and wherein determining the gas flow rate of the oxygen enriched gas comprises determining a first gas flow rate for oxygen enriched gas from the first oxygen concentrator and a second gas flow rate for oxygen enriched gas from the second oxygen concentrator.
